(19) United States
(12) Patent Application Publication
(10) Pub. No.: US 2024/0120075 A1
(43) Pub. Date: Apr. 11, 2024

(54) SYSTEMS AND METHODS FOR DISASTER RELIEF

(71) Applicant: Boomboxdr LLC, Madison, MS (US)

(72) Inventor: Donald Glidewell, Madison, MS (US)

(21) Appl. No.: 18/482,215

(22) Filed: Oct. 6, 2023

Related U.S. Application Data
(60) Provisional application No. 63/414,401, filed on Oct. 7, 2022.

(51) Int. Cl. G16H 40/20; G01C 21/34; G01C 21/36; G16H 10/65; G16H 40/67

(52) U.S. Cl. CPC G16H 40/20; G01C 21/3415; G01C 21/3691; G16H 10/65; G16H 40/67

(57) ABSTRACT

A mobile response unit can include a processing circuit. The processing circuit can include a processor and memory. The memory can store instructions that, when executed by the processor, cause the processor to establish a network to connect with a first user device, receive a request to transfer a resident associated with the displacing facility, retrieve a record associated with the resident, retrieve information associated with a plurality of receiving facilities, identify a receiving facility of the plurality of receiving facilities to receive the resident, establish a connection with a second user device, and transmit one or more signals to cause the second user device to display a user interface including a prompt to accept the request to transfer the resident.

### CROSS-REFERENCE TO RELATED PATENT APPLICATIONS

This application claims the benefit of and priority to U.S. Provisional Patent Application No. 63/414,401 filed on Oct. 7, 2022, the entirety of which is incorporated by reference herein.

### BACKGROUND

Long-term care (“LTC”) facilities provide housing and living assistance for residents. These LTC facilities often rely on electronic records systems to track and manage the care of residents. Natural disasters may require the evacuation of LTC facility residents. LTC facilities are required to maintain patient medical records during an evacuation. However, traditional infrastructure may be unavailable during a natural disaster. Furthermore, in the event of an evacuation of the LTC facility, portable access to the electronic records systems are needed.

### SUMMARY

At least one embodiment is directed to a mobile response unit. The mobile response unit can include a processing circuit. The processing circuit can include a processor and memory. The memory can store instructions that, when executed by the processor, cause the processor to establish a network to connect with a first user device, the first user device associated with a displacing facility. The instructions can also cause the processor to receive, responsive to establishment of the network, from the first user device, a request to transfer a resident associated with the displacing facility. The instructions can also cause the processor to retrieve, from the memory, a record associated with the resident. The instructions can also cause the processor to retrieve, from the memory, information associated with a plurality of receiving facilities. The instructions can also cause the processor to identify, based on the record associated with the resident and the information associated with the plurality of receiving facilities, a receiving facility of the plurality of receiving facilities to receive the resident. The instructions can also cause the processor to establish, via the network, a connection with a second user device. The second user device can be associated with the receiving facility. The instructions can also cause the processor to transmit, via the network, to the second user device, one or more signals to cause the second user device to display a user interface including a prompt to accept the request to transfer the resident.

At least one embodiment is directed to a mobile response unit. The mobile response unit can include a processing circuit. The processing circuit can include a processor and memory. The memory can store instructions that, when executed by the processor, cause the processor to establish a network to connect with a first user device, the first user device associated with a displacing facility. The instructions can also cause the processor to receive, responsive to establishment of the network, from the first user device, a request to transfer a resident associated with the displacing facility. The instructions can also cause the processor to retrieve, from the memory, a record associated with the resident. The instructions can also cause the processor to retrieve, from the memory, information associated with a plurality of receiving facilities. The instructions can also cause the processor to identify, based on the record associated with the resident and the information associated with the plurality of receiving facilities, a receiving facility of the plurality of receiving facilities to receive the resident. The instructions can also cause the processor to establish, via the network, a connection with a second user device. The second user device can be associated with the receiving facility. The instructions can also cause the processor to transmit, via the network, to the second user device, one or more signals to cause the second user device to display a user interface including a prompt to accept the request to transfer the resident. The instructions can also cause the processor to receive, via the network, from the second user device, an indication to accept the request to transfer the resident. The indication can be received responsive to a selection of a selectable element included in the prompt to accept the request to transfer the resident. The instructions can also cause the processor to transmit, via the network, to the second user device, one or more second signals to cause the second user device to display, via the user interface, the record associated with the resident. The record associated with the resident can be displayed without the second user device storing the record associated with the resident.

At least one embodiment is directed to a mobile response unit. The mobile response unit can include a processing circuit. The processing circuit can include a processor and memory. The memory can store instructions that, when executed by the processor, cause the processor to establish a network to connect with a first user device, the first user device associated with a displacing facility. The instructions can also cause the processor to receive, responsive to establishment of the network, from the first user device, a request to transfer a resident associated with the displacing facility. The instructions can also cause the processor to retrieve, from the memory, a record associated with the resident. The instructions can also cause the processor to retrieve, from the memory, information associated with a plurality of receiving facilities. The instructions can also cause the processor to identify, based on the record associated with the resident and the information associated with the plurality of receiving facilities, a receiving facility of the plurality of receiving facilities to receive the resident. The instructions can also cause the processor to establish, via the network, a connection with a second user device. The second user device can be associated with the receiving facility. The instructions can also cause the processor to transmit, via the network, to the second user device, one or more signals to cause the second user device to display a user interface including a prompt to accept the request to transfer the resident. The instructions can also cause the processor to receive, via the network, from the first user device, a request to associate the resident with a wearable record device. The instructions can also cause the processor to establish, responsive to receipt of the request to associate the resident with the wearable record device, via the network, a connection with the wearable record device. The instructions can also cause the processor to provide, via the network, to the wearable record device, the record associated with the resident to cause the wearable record device to store, in memory of the wearable record device, the record associated with the resident. The instructions can also cause the processor to receive, via the network, from the wearable record device, a confirmation to indicate that the resident is associated with the wearable record device.

This summary is illustrative only and is not intended to be in any way limiting. Other aspects, inventive features, and advantages of the devices or processes described herein will become apparent in the detailed description set forth herein, taken in conjunction with the accompanying figures, wherein like reference numerals refer to like elements.

### BRIEF DESCRIPTION OF THE DRAWINGS

FIG. 1 is a perspective view of a mobile response unit, according to an exemplary embodiment.

FIG. 2 is a system diagram of the mobile response unit of FIG. 1, according to an exemplary embodiment.

FIG. 3A is a user interface of the mobile response unit of FIG. 2, according to an exemplary embodiment.

FIG. 3B is a keyboard of the user interface of FIG. 3A having an emergency key, according to an exemplary embodiment.

FIG. 4 is a communication interface of the mobile response unit of FIG. 2, according to an exemplary embodiment.

FIG. 5 is a memory of the mobile response unit of FIG. 2, according to an exemplary embodiment.

FIG. 6 is a user interface of the mobile response unit of FIG. 2, according to an exemplary embodiment.

FIG. 7 is a flow diagram of a method of adding modules to the user interface of FIG. 6, according to an exemplary embodiment.

FIG. 8 is a receiving user interface of the resident displacement manager of FIG. 6, according to an exemplary embodiment.

FIG. 9 is a flow diagram of a method of editing the number of available beds in the receiving user interface of FIG. 8, according to an exemplary embodiment.

FIG. 10 is a displacement user interface of the resident displacement manager of FIG. 6, according to an exemplary embodiment.

FIG. 11A is a first section of a details pane of the displacement user interface of FIG. 5, according to an exemplary embodiment.

FIG. 11B is the second section of the details pane of FIG. 11A, according to an exemplary embodiment.

FIG. 12 is a flow diagram of a set-up method for the resident displacement manager of FIG. 6, according to an exemplary embodiment.

FIG. 13 is a flow diagram of a backup method for the resident displacement manager of FIG. 6, according to an exemplary embodiment.

FIG. 14 is a flow diagram of a method for transferring residents using the resident displacement manager of FIG. 6, according to an exemplary embodiment.

FIG. 15 is an illustration of a graphical user interface usable within the resident displacement manager of FIG. 6, according to an exemplary embodiment.

FIG. 16 is a block diagram of a disaster relief system, according to an exemplary embodiment.

FIG. 17 is a user interface displaying a facility dashboard, according to an exemplary embodiment.

FIG. 18 is a user interface displaying a prompt to request directions, according to an exemplary embodiment.

FIG. 19 is a user interface displaying a prompt to view a route, according to an exemplary embodiment.

FIG. 20 is a user interface displaying an indication to a facility, according to an exemplary embodiment.

FIG. 21 is a user interface displaying a route to a facility, according to an exemplary embodiment.

FIG. 22 is a user interface displaying a prompt to view an updated route, according to an exemplary embodiment.

FIG. 23 is a flow diagram of a method for providing directions to a facility, according to an exemplary embodiment.

FIG. 24 is a block diagram of a system for facilitating communication, according to an exemplary embodiment.

FIG. 25 is a user interface displaying a prompt to view a record, according to an exemplary embodiment.

FIG. 26 is a user interface displaying a prompt to request facility information, according to an exemplary embodiment.

FIG. 27 is a user interface displaying information associated with at least one facility, according to an exemplary embodiment.

FIG. 28 is a user interface displaying a prompt to accept a transfer, according to an exemplary embodiment.

FIG. 29 is a flow diagram of a method for facilitating communication between devices, according to an exemplary embodiment.

FIG. 30 is a block diagram of a system for providing and updating occupant medical records, according to an exemplary embodiment.

FIG. 31 is a user interface displaying a menu, according to an exemplary embodiment.

FIG. 32 is a user interface displaying a window, according to an exemplary embodiment.

FIG. 33 is a user interface displaying a window, according to an exemplary embodiment.

FIG. 34 is a user interface displaying a window, according to an exemplary embodiment.

FIG. 35 is a user interface displaying a window, according to an exemplary embodiment.

FIG. 36 is a flow diagram of a method for associating a mobile record device, according to an exemplary embodiment.

FIG. 37 is a block diagram of system for facilitating communication between one or more facilities, according to an exemplary embodiment.

FIG. 38 is a user interface including a window, according to an exemplary embodiment.

FIG. 39 is a user interface including a window, according to an exemplary embodiment.

FIG. 40 is a user interface including a window, according to an exemplary embodiment.

FIG. 41 is a user interface is a user interface including a window, according to an exemplary embodiment.

FIG. 42 is a user interface including a window, according to an exemplary embodiment.

FIG. 43 is a flow diagram of a process for facilitating the transfer of a resident, according to an exemplary embodiment.

FIG. 44 is a block diagram of a system for monitoring emergency preparedness, according to an exemplary embodiment.

FIG. 45 is a user interface including a window, according to an exemplary embodiment.

FIG. 46 is a user interface including a report, according to an exemplary embodiment.

FIG. 47 is a user interface including a window, according to an exemplary embodiment.

FIG. 48 is a block diagram of a method for generating an emergency preparedness score, according to an exemplary embodiment.

### DETAILED DESCRIPTION

According to an exemplary embodiment, a mobile system for providing access to electronic medical records is described herein.

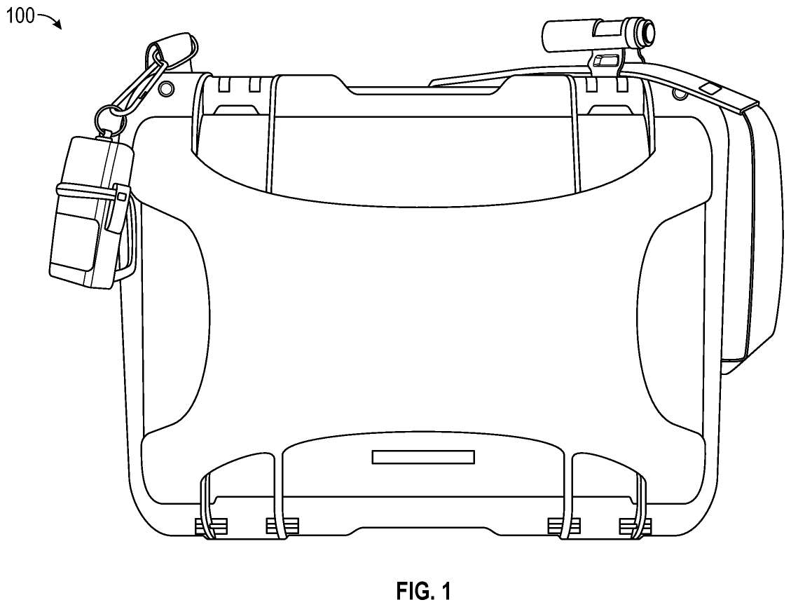

FIG. 1 illustrates mobile response unit 100 for responding to emergencies and/or natural disasters. In some embodiments, mobile response unit 100 is stored at a long-term care (“LTC”) facility and used in the event of emergency and/or natural disaster. In some embodiments, mobile response unit 100 is operable at a LTC facility as a stand-in for conventional systems. Additionally or alternatively, mobile response unit 100 may be portable and/or deployable from remote locations. For example, a nurse may use mobile response unit 100 to continue charting during an evacuation of a LTC facility. In other embodiments, mobile response unit 100 is used in other facilities (e.g., clinics, hospitals, daycares, schools, zoos, etc.). For example, mobile response unit 100 may be used to track the wellbeing of a number of students in a school following an active shooter event. In some embodiments, mobile response unit 100 is durable (e.g., shock-proof, drop resistant, water-proof, etc.) to avoid damage during an emergency.

Mobile response unit 100 simplifies the resident transfer process by automating many tasks associated with resident transfer and improving the transfer process. For example, LTC facility staff may traditionally call each receiving facility individually to determine a number of available beds and/or request the transfer of residents. Furthermore, medical records associated with a transferring resident may be physically printed and transferred with the transferring resident. In addition to simplifying the resident transfer process, mobile response unit 100 supports LTC facilities in complying with government regulations. For example, mobile response unit 100 may locally store medical records (e.g., retrieve and maintain an offline copy, facilitate live charting, etc.) to maintain access to the medical records even during a connectivity outage with the electronic medical record system (e.g., due to natural disaster, etc.). Additionally or alternatively, mobile response unit 100 may facilitate regulatory data compliance. For example, mobile response unit 100 may transmit electronic medical records according to an Occupational Safety and Health Administration (“OSHA”) regulation and/or a Health Insurance Portability and Accountability Act (“HIPAA”) regulation.

Mobile response unit 100 may provide many functions, as described in detail below. For example, mobile response unit 100 may provide a network connection (e.g., internet access, satellite connection, etc.). In some embodiments, the network connection facilitates uninterrupted access to electronic medical records (“EMR”). For example, a user may continue charting using mobile response unit 100. In some embodiments, mobile response unit 100 includes or provides emergency communications systems (e.g., HAM radio, video chat, voice over internet protocol (“VoIP”), television tuner, etc.). Additionally or alternatively, mobile response unit 100 may provide a number of emergency response functions. For example, mobile response unit 100 may provide an emergency contact list, an emergency procedure checklist, and/or an evacuation management system (e.g., resident displacement manager, etc.).

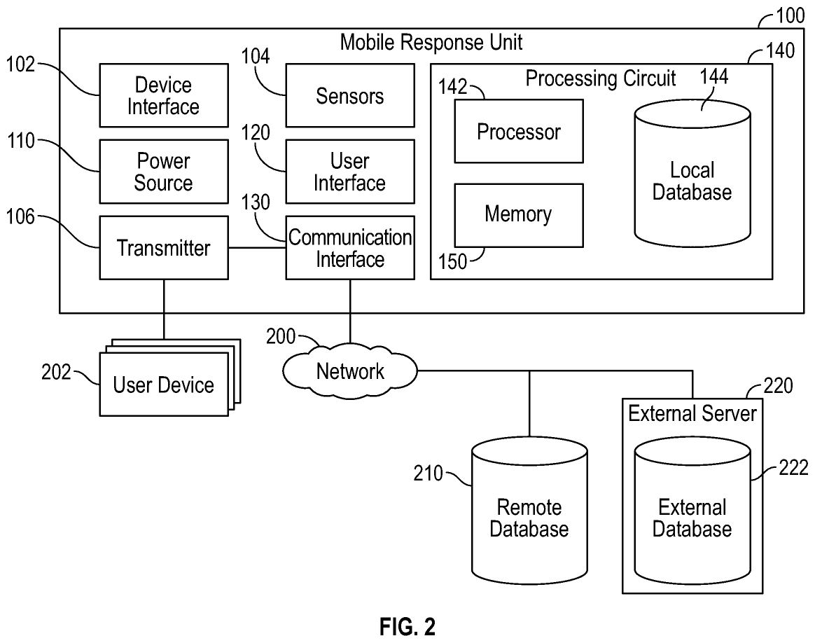

As shown in FIG. 2, mobile response unit 100 includes device interface 102, sensors 104, transmitter 106, power source 110, user interface 120, communication interface 130, and processing circuit 140. In some embodiments, mobile response unit 100 includes a different number, type, combination, and/or configuration of components. For example, mobile response unit 100 may include a visual signal device (e.g., a light, a flare, a balloon, etc.). In some embodiments, device interface 102 facilitates actions between mobile response unit 100 and external devices. In some embodiments, device interface 102 is or includes a number of ports (e.g., serial ports, RS-232, COM port, parallel port, audio port, VGA port, DVI port, RCA, HDMI, universal serial bus (“USB”), firewire, Ethernet (RJ45), modem (RJ14), power connectors, GPIO, etc.) and/or external devices (e.g., switches, buttons, knobs, remotes, etc.). For example, device interface 102 may include a panic alarm button configured to signal an emergency in response to engagement thereof by a user (e.g., an active shooter situation, etc.). In some embodiments, device interface 102 facilitates user interaction with or modification of processing circuit 140 and/or any other component of mobile response unit 100. For example, device interface 102 may include a subscriber identity module (“SIM”) card slot for a user to insert a SIM card to interface with a router of communication interface 130. In some embodiments, device interface 102 is externally accessible to a user. For example, mobile response unit 100 may include an Ethernet port on an external surface. Additionally or alternatively, some or all of the components of device interface 102 may be located internally within mobile response unit 100 to protect them from unauthorized users and/or external elements (e.g., water, dust, etc.). In some embodiments, device interface 102 monitors (e.g., via sensors 104, etc.) the operation of one or more components of mobile response unit 100 (e.g., power source 110, transmitter 106, etc.) and transmits a message in response to an indication that the one or more components of mobile response unit 100 are malfunctioning. For example, device interface 102 may send an email to one or more individuals if transmitter 106 stops working.

According to an exemplary embodiment, sensors 104 measure one or more parameters. For example, sensors 104 may be or include temperature sensors, humidity sensors, air quality sensors, proximity sensors, light sensors, vibration sensors, and/or any other sensors. In some embodiments, mobile response unit 100 is configured to control one or more components thereof based on data from sensors 104. For example, mobile response unit 100 may increase a brightness of a display in response to a light sensor indicating that there is a significant amount of direct sunlight on the display. In some embodiments, sensors 104 include a global positioning system (“GPS”) module. In some embodiments, sensors 104 are configured to detect one or more events. For example, sensors 104 may be configured to detect a gunshot.

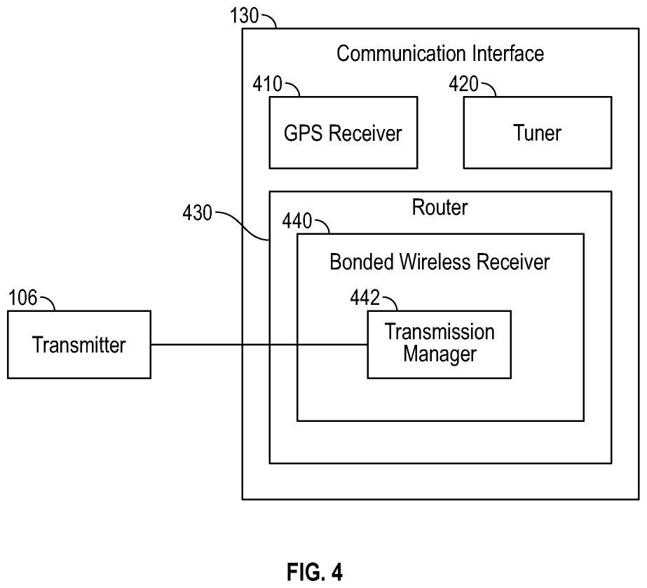

Transmitter 106 facilitates wireless communication between mobile response unit 100 and one or more local devices. For example, mobile response unit 100, via transmitter 106, may broadcast a Wi-Fi or other wireless local area network (“WLAN”) signal to provide Internet access to nearby user devices. Additionally or alternatively, transmitter 106 may transmit data to one or more cellular networks. For example, transmitter 106 may communicate with a Sprint and Verizon cellular network. In some embodiments, transmitter 106 includes a number of transmitters (e.g., five, etc.). Transmitter 106 is discussed in detail below with reference to FIG. 4.

In some embodiments, power source 110 receives and stores electrical power from a power source for future use (e.g., in a remote location where electricity is not readily available, during a power outage, etc.). In some embodiments, power source 110 includes a solar panel system, a combustion generator (e.g., a gasoline-fueled generator, etc.), a power supply (e.g., a 120 Volt (“V”) AC wall charger, a 220V AC wall charger, a 240V AC wall charger, etc.), a 12V car adapter, a battery, and/or an external energy storage source (e.g., an energy tank, a battery, etc.). The stored electrical power may power mobile response unit 100 and/or a load device (e.g., a smartphone, a tablet, an E-reader, a computer, a laptop, a smartwatch, a portable and rechargeable battery pack, appliances, lights, display monitors, an electrical grid of a building, etc.) to at least one of charge and power the load device. In some embodiments, power source 110 protects against fluctuations in external power sources (e.g., electrical surges, interruptions, etc.). For example, power source 110 may be or include an uninterruptible power supply (“UPS”).

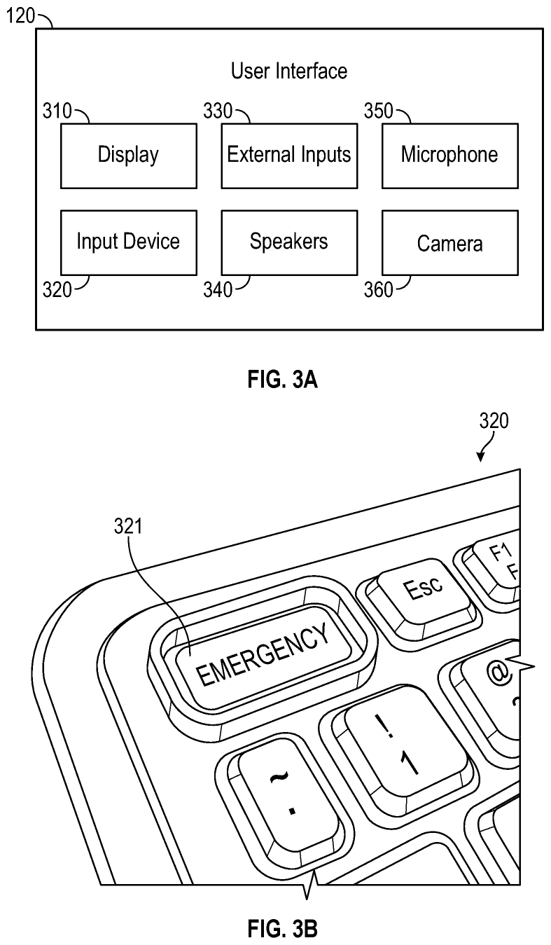

User interface 120 facilitates user interaction with mobile response unit 100. In various embodiments, user interface 120 facilitates users to receive information from and provide information to mobile response unit 100. For example, user interface 120 may be or include a touch-screen display. User interface 120 is discussed in detail below with reference to FIGS. 3A-3B.

Communication interface 130 facilitates communication between mobile response unit 100 and external applications, devices, and/or systems. In some embodiments, communication interface 130 includes wired or wireless interfaces (e.g., jacks, antennas, transmitters, receivers, transceivers, wire terminals, routers, mobile broadband modems, etc.) for conducting data communication with various systems, devices, and/or networks. For example, communication interface 130 may include an Ethernet card and port for sending and receiving data via an Ethernet-based communications network and/or a Wi-Fi transceiver for communicating via a wireless communications network. In some embodiments, communication interface 130 is configured to communicate via local area networks or wide area networks (e.g., the Internet, a building WAN, etc.) and uses a variety of communications protocols (e.g., IP, etc.). In some embodiments, communication interface 130 communicates via network 200. In some embodiments, network 200 is a cellular network and/or a combination of cellular networks. For example, network 200 may be a combination of Verizon, AT&T, and Sprint networks. In various embodiments, communication interface 130 bonds multiple cellular networks together to provide a higher bandwidth signal. Additionally or alternatively, communication interface 130 may be a redundant connection. For example, communication interface 130 may be bonded and/or failover wireless internet connection. In some embodiments, communication interface 130 facilitates connecting to telemedicine support. For example, a user, via communication interface 130, may video chat with a telemedicine professional. In some embodiments, communication interface 130 facilitates sending one or more notifications (e.g., text messages, emails, automated phone calls, etc.) to notify one or more individuals. For example, communication interface 130 may automatedly call one or more family members of a resident of a LTC facility. Communication interface 130 is discussed in detail below with reference to FIG. 4.

Processing circuit 140 includes processor 142, local database 144, and memory 150. In other embodiments, local database 144 may store electronic medical records (“EMR”) and/or other information. In some embodiments, local database 144 stores a local copy of external data (e.g., patient medical records, etc.) in case mobile response unit 100 loses network connection. In some embodiments, local database 144 is part of memory 150. Additionally or alternatively, local database 144 may augment memory 150.

In some embodiments, processor 142 is a general purpose or specific purpose processor, an application specific integrated circuit (ASIC), one or more field programmable gate arrays (FPGAs), a group of processing components, or other suitable processing components. In some embodiments, processor 142 is configured to execute computer code or instructions stored in memory 150 or received from other computer readable media (e.g., CDROM, network storage, a remote server, etc.).

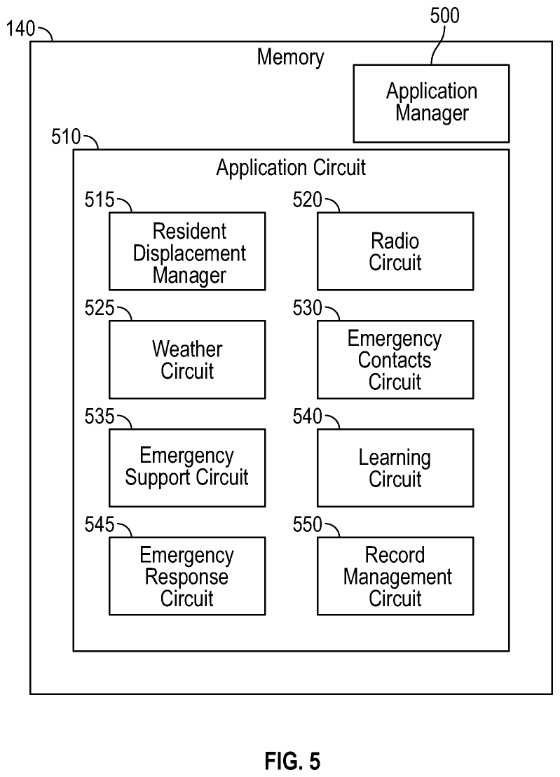

In some embodiments, memory 150 includes one or more devices (e.g., memory units, memory devices, storage devices, etc.) for storing data and/or computer code for completing and/or facilitating the various processes described in the present disclosure. In some embodiments, memory 150 includes random access memory (RAM), read-only memory (ROM), hard drive storage, temporary storage, non-volatile memory, flash memory, optical memory, or any other suitable memory for storing software objects and/or computer instructions. In some embodiments, memory 150 includes database components, object code components, script components, or any other type of information structure for supporting the various activities and information structures described in the present disclosure. In some embodiments, memory 150 is communicably connected to processor 142 via processing circuit 140 and includes computer code for executing (e.g., by processor 142) one or more processes described herein. The functions of some of these modules is described in greater detail below with reference to FIG. 5.

In some embodiments, user device 202 is a smartphone, a tablet, a laptop computer, a desktop computer, and/or any other mobile and/or stationary computing device. For example, user device 202 may be a desktop computer used by health care professionals (e.g., nurses, doctors, etc.) at a LTC facility to record EMRs. In various embodiments, user device 202 facilitates accessing one or more features of mobile response unit 100 remotely. For example, a user using user device 202 may change configuration settings of mobile response unit 100 via an online portal.

In some embodiments, remote database 210 stores information relating to the operation of the mobile response unit 100. For example, remote database 210 may store EMRs. In some embodiments, remote database 210 stores non-critical data such as configuration settings to facilitate storing more critical data locally by mobile response unit 100, in local database 144 for example. In some embodiments, remote database 210 is a copy of an external database (e.g., external database 222, etc.). In some embodiments, remote database 210 is a “cloud” for mobile response unit 100. Additionally or alternatively, remote database 210 may back up data stored on mobile response unit 100. For example, mobile response unit 100 may periodically (e.g., daily, hourly, etc.) copy all data (e.g., memory 150, local database 144, etc.) to remote database 210. Additionally or alternatively, remote database 210 may be configured to retrieve and store data from other sources (e.g., external server 220, external database 222 etc.). For example, remote database 210 may pull EMRs from third party sources (e.g., external database 222, etc.) and store the EMRs to comply with patient privacy regulations. In some embodiments, mobile response unit 100 refreshes the data stored. For example, every three days mobile response unit 100 may retrieve new electronic medical records to replace previously stored electronic medical records.

External server 220 is an external computing system. In some embodiments, external server 220 includes a LTC facility server, an EMR server, a payroll server, and/or any other server or computing system. For example, external server 220 may be a receiving computing system of a LTC facility. In some embodiments, external server 220 includes external database 222. External database 222 may include emergency contacts, emergency procedures, EMRs, payroll data and/or any other data. For example, external database 222 may include electronic medication administration records (“eMAR”), electronic treatment authorization request records (“eTAR”), and/or activities of daily living records (“ADL”). In various embodiments, external database 222 is a number of different databases. For example, a first external database 222 may store payroll data for a LTC facility, while a second external database 222 may store EMRs relating to LTC facility residents. In some embodiments, external database 222 is separate of external server 220.

As shown in FIG. 3A, user interface 120 includes display 310, input device 320, external inputs 330, speakers 340, microphone 350, and camera 360. In some embodiments, user interface 120 includes a different number and/or type of components. Display 310 presents visual information to a user. In some embodiments, display 310 is an electroluminescent (“ELD”) display, a liquid crystal display (“LCD”), a light-emitting diode (“LED”) display, an organic light-emitting diode (“OLED”) display, a plasma (“PDP”) display, a quantum dot (“QLED”) display, an E-paper display, and/or any other display technology known in the art. In some embodiments, display 310 is a touchscreen display. Additionally or alternatively, display 310 may include a touch element. For example, display 310 may include a resistive touchscreen, a surface acoustic wave touchscreen, a capacitive touchscreen, an infrared grid touchscreen, and/or another type of touchscreen.

Input device 320 facilitates a user to provide input to mobile response unit 100. For example, input device 320 may be a keyboard, mouse, trackpad, track ball, joystick, light pen, and/or any other input device known in the art. In some embodiments, input device 320 is a single input device (e.g., a keyboard, etc.). Additionally or alternatively, input device 320 may be a combination of input devices (e.g., keyboard and mouse, etc.). External inputs 330 provide additional user inputs beyond input device 320. In various embodiments, external inputs 330 facilitate additional peripheral devices to be connected to mobile response unit 100 to expand the functionality of mobile response unit 100. In some embodiments, external inputs 330 facilitate for bulky peripheral devices to be connected to mobile response unit 100 that would otherwise not fit within the form factor of mobile response unit 100. For example, a bank of landline telephones may be connected as external inputs 330. In some embodiments, external inputs 330 include a panic alarm button. For example, a panic alarm button may facilitate a user in a remote location to send a distress signal to mobile response unit 100. In some embodiments, external inputs 330 include a handheld phone (e.g., via a RJ11 connector, etc.). In some embodiments, external inputs 330 interface with or include any of the components (e.g., Ethernet, GPIO, etc.) of device interface 102.

Microphone 350 is configured to detect and receive audio signals. In some embodiments, microphone 350 receives audio input from a user. For example, a user may interact with microphone 350 to audio-chat with an emergency response team over a VoIP connection. In some embodiments, microphone 350 provides data relating to whether a user of mobile response unit 100 is authorized. For example, mobile response unit 100 may detect an authorized user's voice signature via microphone 350. Camera 360 is used to receive visual data. In some embodiments, camera 360 allows a user to video-chat with an emergency response team. Camera 360 may capture video and/or still images.

As shown in FIG. 3B, input device 320 includes a keyboard having an emergency key 321, according to an exemplary embodiment. For example, a user may use emergency key 321 to indicate an emergency. In some embodiments, emergency key 321 supplements and/or replaces an emergency touchscreen option on display 310. In some embodiments, use of emergency key 321 automatically contacts one or more emergency personnel. The emergency personnel may include public emergency responders (e.g., law enforcement, fire department, a “911” operator, etc.) and/or may include private response coordinators functioning as part of a first response team (e.g., staff trained to assess an emergency situation and coordinate a response, etc.). Additionally or alternatively, emergency key 321 may initiate a video and/or audio chat with one or more support individuals. For example, emergency key 321 may initiate an audio chat with an emergency dispatcher.

As shown in FIG. 4, communication interface 130 includes GPS receiver 410, tuner 420, and router 430. In other embodiments, GPS receiver 410 is configured to receive geolocation and time information from a global navigation satellite system. In some embodiments, GPS receiver 410 determines a position of mobile response unit 100 based on the received signals. For example, GPS receiver 410 may triangulate a position of mobile response unit 100 based on comparing geolocation position and time information received from several GPS satellites. Tuner 420 receives and/or decodes radio, television, and other over-the-air signals. Tuner 420 may include an antenna or other signal capture device to receive over-the-air signals. Additionally or alternatively, tuner 420 may encode and/or transmit radio, television, and other over-the-air signals. For example, tuner 420 may be configured to send and receive signals as part of a HAM radio. In some embodiments, tuner 420 receives local television broadcasts to be displayed (e.g., via display 310, etc.) by mobile response unit 100. In some embodiments, communication interface 130 includes a visual beacon. For example, communication interface 130 may facilitate connection of a helium balloon having a programmable illumination beacon. In some embodiments, the visual beacon may float above the ground as to be visible at a distance and may include an antenna and/or other transmitting and/or receiving component (e.g., GPS receiver, etc.) to facilitate communication with one or more external devices.

Router 430 directs data packets between computer networks. In some embodiments, router 430 connects to a variety of networks (e.g., LAN, WAN, the Internet, cellular networks, etc.). In some embodiments, router 430 connects one or more local devices (e.g., user device 202, etc.) to an external network (e.g., network 200, the Internet, etc.). Additionally or alternatively, router 430 may bond one or more cellular network connections together to provide a wireless network connection to mobile response unit 100. In some embodiments, router 430 transmits a Wi-Fi signal. Additionally or alternatively, router 430 may connect (e.g., via an Ethernet connection, etc.) to existing network infrastructure to provide Internet access. For example, router 430 may interface with an existing router of a LTC facility that has lost Internet access in order to restore Internet access to the LTC facility via a bonded cellular connection. In some embodiments, router 430 provides a redundant network connection (e.g., a failover Internet connection, etc.). In some embodiments, router 430 includes wireless receiver 440.

Wireless receiver 440 wirelessly communicates data over one or more network channels. In some embodiments, the network channels are cellular network channels. In some embodiments, the network channels are a mix of cellular network channels, conventional network channels, (e.g., physical transmission lines, WLAN, etc.), and/or other channels (e.g., Bluetooth, etc.). In some embodiments, wireless receiver 440 bonds (e.g., combines, transmits in parallel, etc.) multiple network channels simultaneously to provide a higher bandwidth connection than otherwise available. Additionally or alternatively, wireless receiver 440 may seamlessly transfer between network channels to provide an uninterrupted connection in the event that one or more networks lose connection.

In some embodiments, wireless receiver 440 includes and/or is coupled to one or more transmitters (e.g., transmitter 106, etc.) to transmit data to one or more cellular networks. For example, wireless receiver 440 may include a 0.6 Watt transmitter for local transmission and a 3 Watt transmitter for extended transmission. In some embodiments, the one or more transmitters transmit on one or more frequencies and/or frequency bands (e.g., 890-915 MHz, etc.). In some embodiments, a load supervisor, shown as transmission manager 442, monitors the transmission load for each transmitter and/or the router 430. For example, the number of channels may change based on current demand. For example, a Verizon network channel and a conventional network channel may be bonded to provide connection during low traffic periods, while a Verizon network channel, a Sprint network channel, and a conventional network channel may be bonded to provide a high bandwidth connection during peak traffic periods.

In various embodiments, wireless receiver 440 and/or router 430 include one or more cellular networking components (e.g., SIM cards, long-term-evolution (“LTE”) adapters, etc.). In some embodiments, wireless receiver 440 includes one or more different cellular networking components, each configured to communicate on a different cellular network. For example, a LTE router may have three SIM cards, each for different cellular network carriers.

In some embodiments, transmission manager 442 is configured to allocate channels not currently in use to increase the quality of service. In some embodiments, transmission manager 442 sends packets in parallel through separate cellular, Bluetooth, and/or WLAN pathways. For example, if a Bluetooth or WLAN connection is established, transmission manager 442 may immediately route packets using the appropriate connection standard. In some embodiments, transmission manager 442 pings its environment to decide on optimal transmission medium. For example, if the signal reception is poor for a first WLAN pathway, transmission manager 442 may send some packets in parallel through both the primary WLAN pathway and a secondary communication channel (cellular, Bluetooth, and/or WLAN) to make sure some of the packets arrive at their destinations. In some embodiments, if the signal strength is adequate, transmission manager 442 prefers Bluetooth and/or WLAN pathways to minimize the number of user devices using the expensive cellular system.

As shown in FIG. 5, memory 150 includes application manager 500 and application circuit 510, according to an exemplary embodiment. Application manager 500 manages operation of mobile response unit 100. In some embodiments, application manager 500 is configured to facilitate customization of the look and feel of the display interface of mobile response unit 100. For example, via application manager 500 a user may select from a number of applications or functionalities (e.g., functions provided by application circuit 510, etc.) to be displayed on a home screen. In some embodiments, application manager 500 is accessible via an online portal (e.g., a web application, etc.). In some embodiments, application manager 500 supports a remote desktop protocol (“RDP”) connection to connect mobile response unit 100 with one or more other devices and/or services.

Application circuit 510 is embedded and/or locally executed (e.g., by mobile response unit 100, etc.) applications. For example, application circuit 510 may include embedded web applications. In some embodiments, application manager 500 facilitates a user to import new functions (e.g., components of application circuit 510) and/or select from already-loaded functions. For example, a user may embed a new function (e.g., component of application circuit 510) via a HTML link. This facilitates application manager 500 to be customizable and adapt to unforeseen needs. For example, a user could embed a payroll web application to manage payroll via mobile response unit 100. In some embodiments, functions (e.g., components) of application circuit 510 are stored locally on mobile response unit 100 (e.g., in memory 150, etc.). Additionally or alternatively, functions of application circuit 510 may be stored in remote database 210.

As shown in FIG. 5, application circuit 510 includes resident displacement circuit 515, radio circuit 520, weather circuit 525, emergency contacts circuit 530, emergency support circuit 535, learning circuit 540, emergency response circuit 545, and record management circuit 550. In other embodiments, mobile response unit 100 includes a different number, type, combination, and/or configuration of functions/circuits.

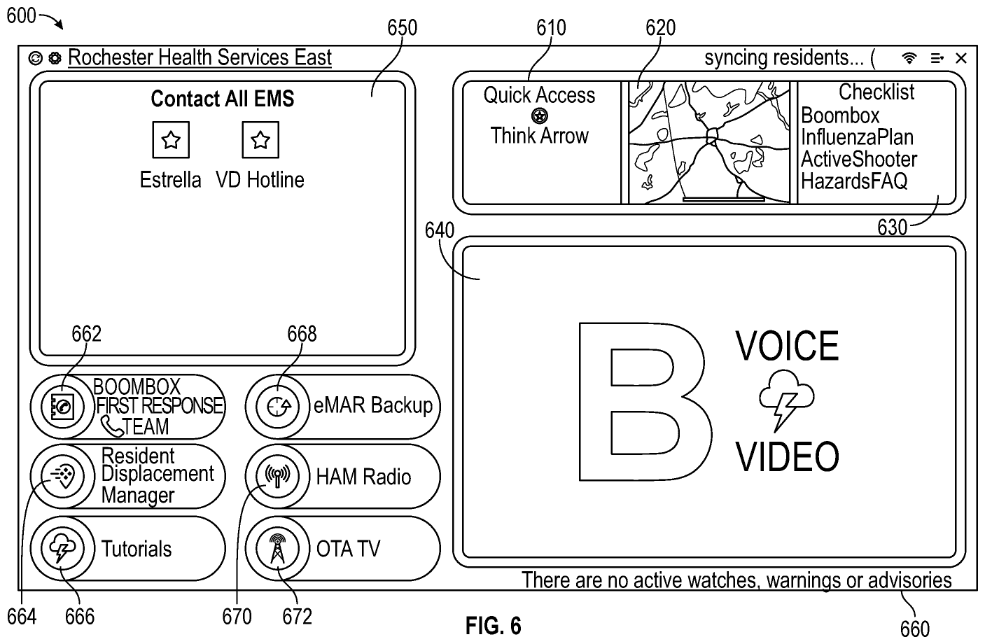

Resident displacement circuit 515 facilitates a user to track and manage a location of one or more individuals associated with a LTC facility. In various embodiments, resident displacement circuit 515 is configured to manage the relocation of LTC residents and/or staff during an emergency and/or natural disaster. For example, via resident displacement circuit 515 a user may assign LTC residents and staff to one or more different LTC evacuation facilities. For example, a LTC facility or other institution capable of providing care for LTC residents may indicate a number of available beds and a user using resident displacement circuit 515 may assign, via an interface, one or more LTC residents to the available beds. For example, a first LTC facility in Fort Lauderdale may indicate via a website a number of available beds and a second LTC facility experiencing a natural disaster in Tampa may assign its residents to the available beds via resident displacement circuit 515. Resident displacement circuit 515 is described in detail below with reference to FIGS. 6-15.

Radio circuit 520 provides radio communication functionality. In some embodiments, radio circuit 520 is a HAM radio. Additionally or alternatively, radio circuit 520 may be a web-based radio (e.g., a web-based HAM radio, etc.). For example, radio circuit 520 may launch a web-based HAM radio controller on mobile response unit 100. In some embodiments, radio circuit 520 transmits and/or receives via a radio antenna coupled to and/or included with mobile response unit 100. For example, radio circuit 520 may connect to an external radio antenna and transmit via the external radio antenna. Weather circuit 525 displays information related to weather conditions. In some embodiments, weather circuit 525 determines a location of mobile response unit 100 (e.g., via GPS receiver 410, etc.) and displays weather conditions based on the location. In some embodiments, weather circuit 525 uses one or more third party weather services. For example, weather circuit 525 may display real time satellite imagery of local weather conditions. In some embodiments, weather circuit 525 predicts the path of a weather element (e.g., a thunderstorm, a flood, a tornado, etc.) and alerts a user if they are in the predicted path. In some embodiments, weather circuit 525 is configured to detect one or more storm patterns. For example, weather circuit 525 may predict if a tornado is likely to touchdown nearby.

Emergency contacts circuit 530 includes contact information for one or more emergency contacts. In some embodiments, emergency contacts circuit 530 includes emergency contacts associated with the LTC facility. For example, emergency contacts circuit 530 may include the phone number of the manager of the LTC facility. In some embodiments, users customize the information included in emergency contacts circuit 530. In some embodiments, the information in emergency contacts circuit 530 is populated from a cloud-based database (e.g., remote database 210, etc.). In some embodiments, emergency contacts circuit 530 provides a phone number, address, email, or other information for each emergency contact. In some embodiments, emergency contacts are organized in a hierarchy. Additionally or alternatively, emergency contacts may be organized by emergency type. For example, contacts related to a natural disaster may be grouped together, and contacts related to an active shooter may be grouped together. In some embodiments, emergency contacts circuit 530 includes contact information for various government emergency lines. For example, emergency contacts circuit 530 may include the local non-emergency phone number.

In some embodiments, emergency support circuit 535 is configured to connect directly to a member of a first response team. In some embodiments, emergency support circuit 535 is coupled to (e.g., notifies devices based on data from, triggered by, etc.) emergency key 321. In some embodiments, emergency support circuit 535 facilitates a user to easily call, message, video chat, or otherwise contact emergency support personnel. For example, emergency support circuit 535 may automatically initiate a video chat with a member of a first response team. In some embodiments, emergency support circuit 535 is configured to follow an emergency procedure. For example, emergency support circuit 535 may reference a cloud-based database (e.g., external database 222, etc.) to determine an appropriate course of action. In some embodiments, emergency support circuit 535 is coupled to emergency contacts circuit 530. For example, a user may select a contact from emergency contacts circuit 530 to add to an automatic call list of emergency support circuit 535. Additionally or alternatively, emergency support circuit 535 may automatically open and initiate a call when a user selects a contact from emergency contacts circuit 530.

In some embodiments, learning circuit 540 facilitates accessing emergency preparedness (“EP”) training materials. In some embodiments, EP training materials include video tutorials. In some embodiments, learning circuit 540 implements a learning plan and can track individuals' certifications and/or learning progress. For example, learning circuit 540 may require a user to complete an active shooter training and may track the user's progress through the training. Additionally or alternatively, learning circuit 540 may include manuals and/or descriptions on the functions and operation of mobile response unit 100.

Emergency response circuit 545 displays one or more emergency response plans. In some embodiments, emergency response circuit 545 includes one or more checklists and/or procedures. For example, emergency response circuit 545 may display a list of text links and/or other graphical response plans. In some embodiments, a user customizes the emergency response plans presented by emergency response circuit 545. Additionally or alternatively, emergency response circuit 545 may automatically pull the emergency response plans from a cloud-based database (e.g., external database 222, etc.). In some embodiments, the emergency response plans include static content (e.g., text documents, pictures, etc.) and/or dynamic content (e.g., web based applications, etc.).

Record management circuit 550 is configured to manage the retrieval and storage of data from external server 220 and/or external database 222. In some embodiments, record management circuit 550 is coupled to local database 144 and/or remote database 210. In some embodiments, record management circuit 550 interfaces with any of the modules and/or components of mobile response unit 100. For example, record management circuit 550 may automatically retrieve EMR data associated with residents of a LTC facility from a third party EMR provider serve and store the EMR data in local database 144 and/or remote database 210. In some embodiments, record management circuit 550 periodically (e.g., daily, hourly, etc.) backs up mobile response unit 100 to remote database 210. In various embodiments, a record management circuit 550 is user-customizable to determine what data is retrieved, stored, and/or backed up.

Speaking now generally, a resident displacement manager is described for managing the transfer of LTC facility residents and the maintenance of associated medical records. The resident displacement manager is configured to support LTC facility staff in transferring residents to other LTC facilities. The resident displacement manager enables other LTC facilities (e.g., receiving facilities, etc.) to indicate openings (e.g., available beds, etc.) and/or accept residents for transfer. LTC facility staff at the displacing facility (e.g., LTC facilities transferring residents to a receiving facility, etc.) review a list of receiving facilities and assign residents to the receiving facilities. The resident displacement manager transfers the medical records associated with the transferred resident to the receiving facility. In other embodiments, the resident displacement manager of the present application is used in other facilities (e.g., clinics, hospitals, daycares, schools, zoos, etc.). For example, the resident displacement manager may be used to track the wellbeing of a number of students in a school following an active shooter event.

The resident displacement manager of the present disclosure simplifies the resident transfer process by automating many tasks associated with resident transfer and improving the transfer process. For example, LTC facility staff may traditionally call each receiving facility individually to determine a number of available beds and/or request the transfer of residents. Furthermore, medical records associated with a transferring resident may be physically printed and transferred with the transferring resident. In addition to simplifying the resident transfer process, the resident displacement manager of the present disclosure supports LTC facilities in complying with government regulations. For example, the resident displacement manger may locally store medical records (e.g., retrieve and maintain an offline copy, facilitate live charting, etc.) to maintain access to the medical records even during a connectivity outage with the electronic medical record system (e.g., due to natural disaster, etc.). Additionally or alternatively, the resident displacement manager of the present disclosure may facilitate regulatory data compliance. For example, the resident displacement manager may transmit electronic medical records according to an Occupational Safety and Health Administration (“OSHA”) regulation and/or a Health Insurance Portability and Accountability Act (“HIPAA”) regulation. In some embodiments, the resident displacement manager of the present disclosure monitors the operation of one or more components of the resident displacement manager (e.g., a processing circuit, a fan, etc.) and notifies one or more individuals based on determining an anomaly in the operation. For example, a monthly report including a list specifying the operational status of each component of the resident displacement manager may be sent to one or more individuals.

Referring now to FIG. 6, user interface 600 of mobile response unit 100 is shown, according to an exemplary embodiment. In various embodiments, user interface 600 is displayed by display 310. In some embodiments, user interface 600 is a home screen of mobile response unit 100. In some embodiments, user interface 600 is customizable via application manager 500, as described in detail with reference to FIG. 5. User interface 600 includes resident displacement manager 664. User interface 600 includes information and functions to assist LTC staff in the event of a natural disaster or emergency. For example, user interface 600 may include emergency contacts and/or the ability to initiate a phone call with the emergency contacts. In some embodiments, user interface 600 is a user interface of an emergency device (e.g., a mobile response unit, etc.) for responding to natural disasters and/or emergencies. Additionally or alternatively, user interface 600 may be standalone. For example, user interface 600 may be a web application that is accessible on any computing device. In some embodiments, user interface 600 includes one or more functionalities 610-650, and a number of buttons 662-672. Functionalities 610-650 may be native applications (e.g., located on and executed by a mobile response unit, etc.). Additionally or alternatively, functionalities 610-650 may be embedded applications (e.g., widgets, etc.). In some embodiments, functionalities 610-650 are customizable (e.g., relocated, added, removed, edited, etc.) to change the appearance and/or functionality of user interface 600. Functionalities 610-650 include quick access 610, weather 620, emergency response procedure 630, emergency support 640, and/or emergency contacts 650. In some embodiments, functionalities 610-650 include a different number, type, and/or combination of functionalities 610-650. In various embodiments, functionalities 610-650 and/or buttons 662-672 correspond to components of application circuit 510. For example, resident displacement manager 664 may correspond to resident displacement circuit 515.

Buttons 662-672 provide additional functionality to functionalities 610-650. In some embodiments, buttons 662-672 represent functionalities 610-650. For example, one of buttons 662-672 may replace emergency contacts 650. In various embodiments, selection of buttons 662-672 launches one or more applications. For example, selection of resident displacement manager 664 may launch resident displacement manager 664. Buttons 662-672 are customizable as described in detail with reference to modules 610-650. Buttons 662-672 include support button 662, resident displacement manager 664, tutorials button 666, eMAR backup button 668, HAM radio button 670, and/or antenna television button 672. In some embodiments, buttons 662-672 include a different number, type, and/or combination of buttons 662-672.

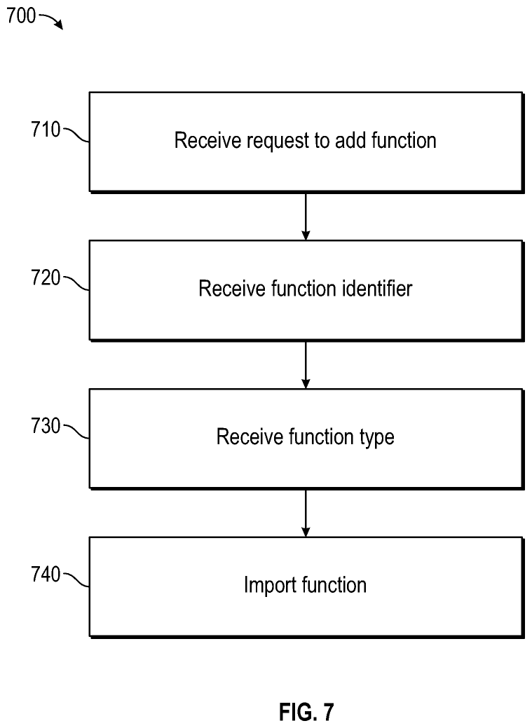

In some embodiments, as shown in FIG. 7, a method 700 of adding functions to user interface 600 is used to add new functionality to user interface 600. For example, a user may embed a web application in user interface 600 via method 700. In other embodiments, the system does not permit the user to add features or functions to, remove features or functions from, customize, or otherwise change the user interface 600. At step 710, user interface 600 receives a request to add a function (e.g., functionalities 610-650, etc.). In various embodiments, a user selects a configuration option of user interface 600 in order to initiate method 700 (i.e., step 710). At step 720, user interface receives a function identifier. In various embodiments, a user inputs a function identifier in the form of a HTML link to a web application. Additionally or alternatively, a user may provide a system path to the location in memory of the function. In some embodiments, user interface 600 is configured to detect and/or adjust to the type of identifier provided. For example, a first identifier specifying a download location for a function may download the function while a second identifier specifying a web based application may scale and embed the application.

At step 730, user interface 600 receives a function type. In some embodiments, a user indicates the function type by selecting the module type from a list. Additionally or alternatively, user interface 600 may automatically determine a function type. The function type describes the functionality of the function. For example, a first weather function may have a “news” type while a second emergency contacts function may have a “contacts” type. In various embodiments, user interface 600 adjusts method 700 based on the type of function. For example, a “news” type function may be imported as a scrolling ticker at the bottom of user interface 600 while a “contacts” type function may be imported as a function option (e.g., functionalities 610-650, etc.) on user interface 600. Additionally or alternatively, a user may customize the function prior to import. For example, the user may select a location and appearance of the function. In other embodiments, user interface 600 automatically determines a function location.

At step 740, the function is imported into user interface 600. In some embodiments, step 740 includes setup of the function. For example, specifying a location for a weather function or a list of contacts for a contacts function. In some embodiments, step 740 results in the function being displayed on user interface 600. Additionally or alternatively, step 740 may result in the function being added to a list of available functionalities that can be subsequently be used.

### Resident Displacement Manager

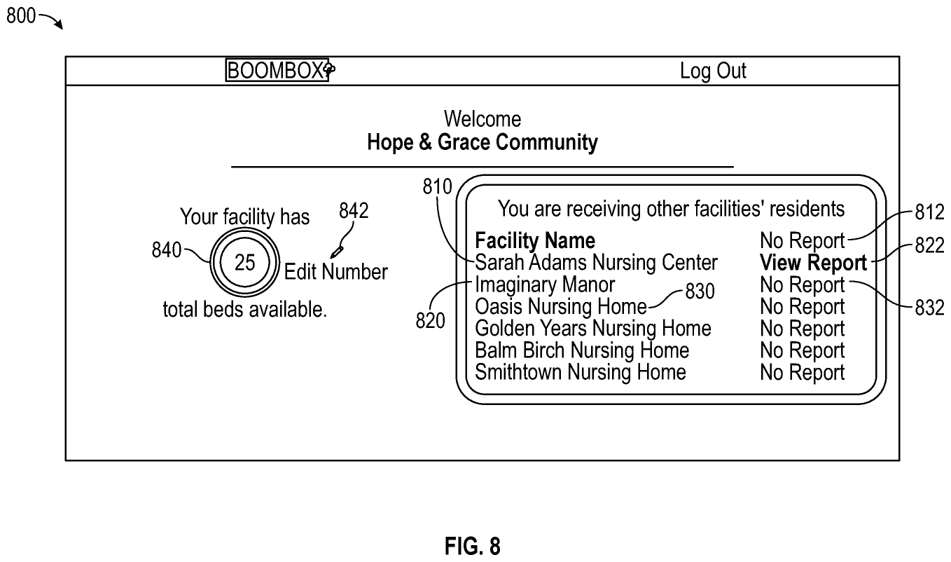

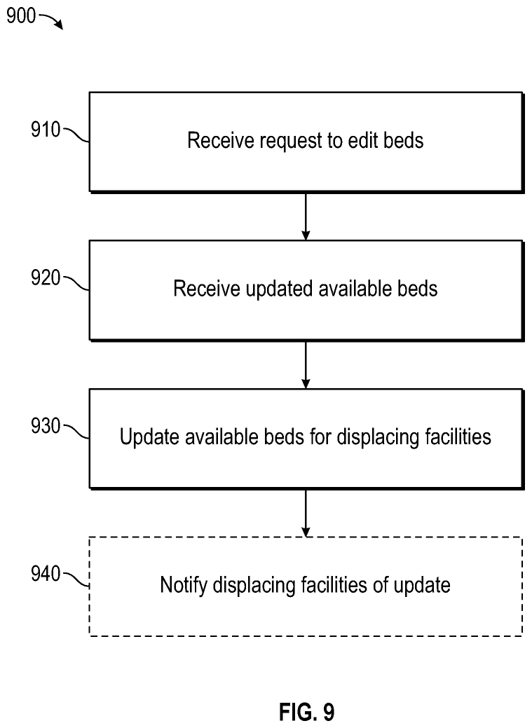

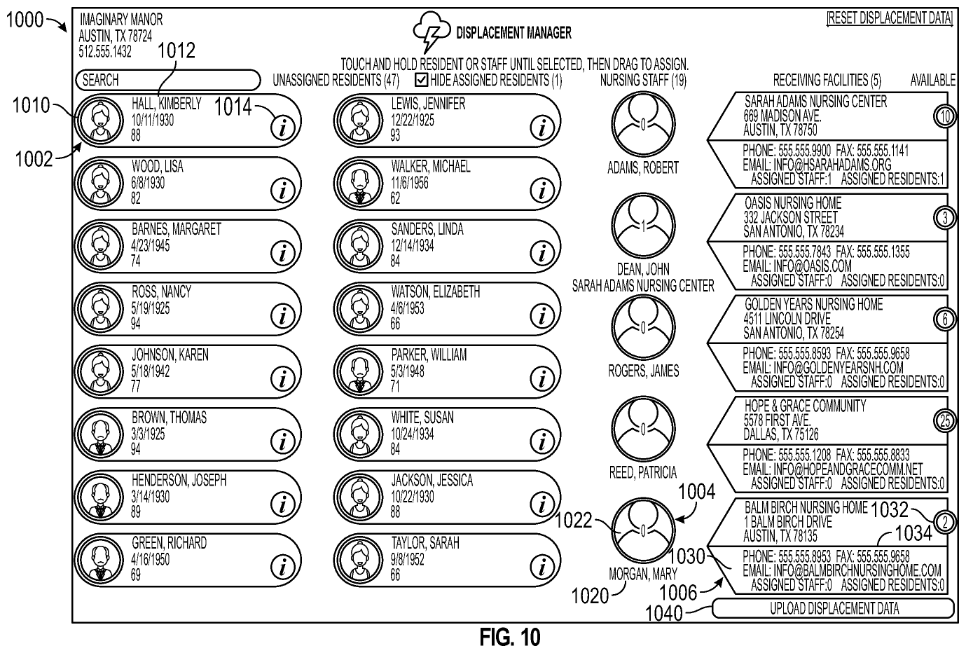

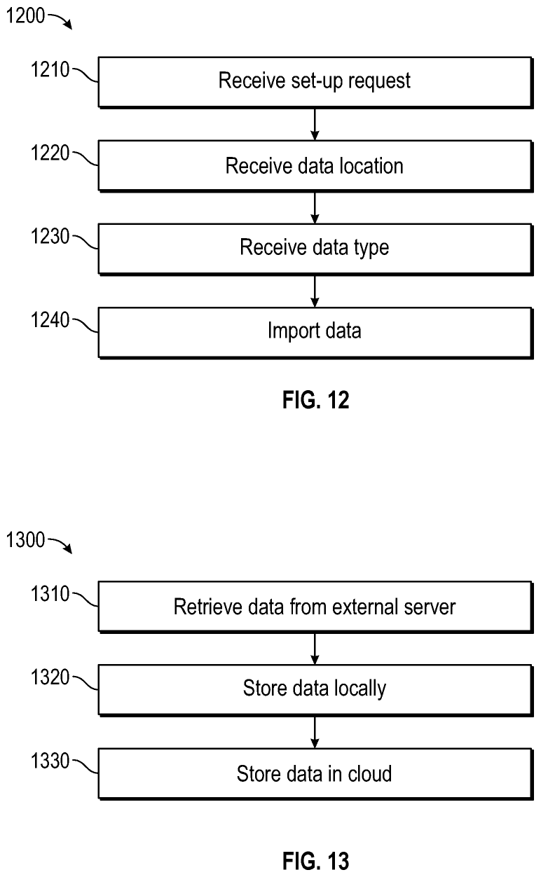

In some embodiments, resident displacement manager 664 part of a larger emergency response solution (e.g., user interface 600, mobile response unit 100, etc.). Additionally or alternatively, resident displacement manager 664 may be a standalone system. For example, resident displacement manager 664 may be accessible as a web application on a smart phone. In various embodiments, resident displacement manager 664 includes a receiving user interface 800, as described in detail with reference to FIGS. 8-9, a displacement user interface 1000, as described in detail with reference to FIGS. 10-12, and/or one or more back-end systems, as described in detail with reference to FIGS. 13-15. In some embodiments, resident displacement manager 664 facilitates live mobile charting (e.g., reviewing, adding, modifying, etc.) of medical records.

Although elements of the present disclosure are described in terms of user interfaces (e.g., user interface 600, resident displacement manager 664, etc.), it should be understood that the systems and methods described herein may be and/or be implemented on and/or at least partially by one or more computing devices (e.g., circuits, servers, databases, microprocessors, etc.). In some embodiments, the systems and methods described herein are implemented as computer instructions (e.g., machine code, etc.) and cause one or more processors, processing circuits, and/or computing devices to carry out details described herein.

Turning now to FIG. 8, receiving user interface 800 of the resident displacement manager is shown, according to an exemplary embodiment. In some embodiment, receiving user interface 800 is accessible from resident displacement manager 664 of user interface 600. Additionally or alternatively, receiving user interface 800 may be independent of user interface 600 (e.g., a standalone web application, etc.). For example, receiving user interface 800 may be accessible from a website and/or a mobile device application. In some embodiments, receiving user interface 800 facilitates one or more receiving facilities to indicate a number of available beds to receive residents. For example, in the event of a natural disaster (e.g., a hurricane, etc.) in Tampa, a LTC facility in Fort Lauderdale could use receiving user interface 800 to indicate to LTC facilities in Tampa how many residents they are able to receive. Furthermore, receiving user interface 800 facilitates receiving facilities to review the individual residents being transferred. For example, using receiving user interface 800, a receiving facility may view a list of which residents they are receiving. Additionally or alternatively, receiving user interface 800 may display and/or enable access to the medical records associated with the residents they are receiving. In various embodiments, resident displacement manager 664 tabulates (e.g., adds, combines, calculates, etc.) the total number of available beds by combining the number of available beds at each receiving facility. In some embodiments, resident displacement manager 664 facilitates LTC facilities to transfer LTC staff to other LTC facilities. For example, a displacing facility may transfer three residents and three health care professionals associated with the three residents to a receiving facility. A receiving facility may be able to view information about the health care professionals (e.g., name, photographs, certifications, license, specialties, etc.).

In some embodiments, the number of available beds is based on location. For example, the resident displacement manager may facilitate an indication, by displacing facilities and/or receiving facilities, of a distance threshold, and the resident displacement manager may calculate a number of available beds based on the distance threshold. As a concrete example, a first receiving facility may indicate they have 18 available beds and can receive residents from up to 25 miles away. A second receiving facility may indicate they have 12 available beds and can receive residents from up to 10 miles away. A first displacing facility may be able to transfer residents up to 15 miles away and be located 9 miles from both the first receiving facility and the second receiving facility. The first displacing facility may see that there are 30 available beds. Meanwhile, a second displacing facility may be able to transfer residents 17 miles and located 9 miles from the first receiving facility and 18 miles from the second receiving facility. The second displacing facility may see that there are 18 beds available.

Receiving user interface 800 includes a number of receiving facilities 810-830 and a number of available beds 840. Available beds 840 indicate the number of available beds the receiving facility has indicated they have available. In some embodiments, displacing facilities request an indication of a number of available beds from one or more receiving facilities. For example, a first displacing facility may have a mutual occupancy agreement with a second receiving facility. In the event of a natural disaster, the first displacing facility may request that the second receiving facility indicate a number and type (e.g., male beds, female beds, life support system equipped beds, dementia ward beds, intensive care unit beds, etc.) of available beds. In various embodiments, available beds 840 is unique to each receiving facility. Available beds 840 includes edit option 842. Edit option 842 facilitates a receiving facility to change the number of beds they indicate they have available. A method of changing the number of available beds is described in detail below with reference to FIG. 9.

Receiving facilities 810-830 lists the names and/or other information associated with each displacing facility that is transferring residents to the receiving facility. In some embodiments, receiving facilities 810-830 lists the number of residents to be received from each receiving facility. Receiving facilities 810-830 includes details 812-832. Details 812-832 provides a user with in-depth information associated with the residents to be transferred. For example, details 812-832 may include a list of the names of each resident being transferred and a link to each resident's medical records.

As shown in FIG. 9, a method 900 is employed to edit the number of available beds. At step 910, receiving user interface 800 receives a request to edit the number of available beds (e.g., available beds 840, etc.). In various embodiments, a user selects edit option 842 to initiate method 900 (e.g., step 910). In some embodiments, step 910 includes opening a new pane and/or dialog to prompt the user and/or receive user input. For example, step 910 may include a dialog: “How many available beds do you have? ______” and a field to receive user input associated with the dialog. It should be understood that although method 900 is described in terms of receiving user interface 800 it is meant in no way to be limiting, and method 900 may additionally or alternatively be carried out, in part or in full, by resident displacement manager 664 and/or any other element described herein.

At step 920, receiving user interface 800 receives an updated number of available beds. In various embodiments, step 920 includes a user submitting an updated number of available beds to a dialog and/or input field. In some embodiments, receiving user interface 800 automatically pulls a number of available beds from a computing system associated with the receiving facility. For example, receiving user interface 800 may query the facility occupancy from a server that manages resident occupancy for the receiving facility. In some embodiments, receiving user interface 800 submits a GET request and/or any other HTTP request to a server associated with the receiving facility.

At step 930, receiving user interface 800 updates a number of available beds displayed to displacing facilities. In various embodiments, step 930 changes a number of available beds displayed to displacing facilities. In some embodiments, step 930 includes retabulating (e.g., adding, calculating, etc.) the number of available beds for each displacing facility. In some embodiments, method 900 includes step 940. At step 940, receiving user interface 800 notifies displacing facilities of the update to the number of available beds. The notification may be a push notification, an automated phone call, a text message, an email, an alert on user interface 600 and/or any other notification. In various embodiments, receiving user interface 800 determines if a displacing facility has assigned a resident to transfer to a bed that is no longer available (e.g., because a receiving facility has reduced the number of available beds, etc.) and notifies the displacing facility.

Turning now to FIG. 10, displacement user interface 1000 for managing the transfer of LTC residents and staff is shown, according to an exemplary embodiment. In some embodiments, displacement user interface 1000 is part of user interface 600 (e.g., is or is included in resident displacement manager 664, etc.). In some embodiments, displacement user interface 1000 is part of a larger resident displacement manager (e.g., resident displacement manager 664, etc.). In various embodiments, displacement user interface 1000 is paired with receiving user interface 800. Additionally or alternatively, displacement user interface 1000 may be standalone (e.g., included in a mobile response unit separate of receiving user interface 800, etc.). For example, displacement user interface 1000 may be included in a mobile response unit for natural disaster and/or emergency relief while receiving user interface 800 may be a web based application accessible via the Internet.

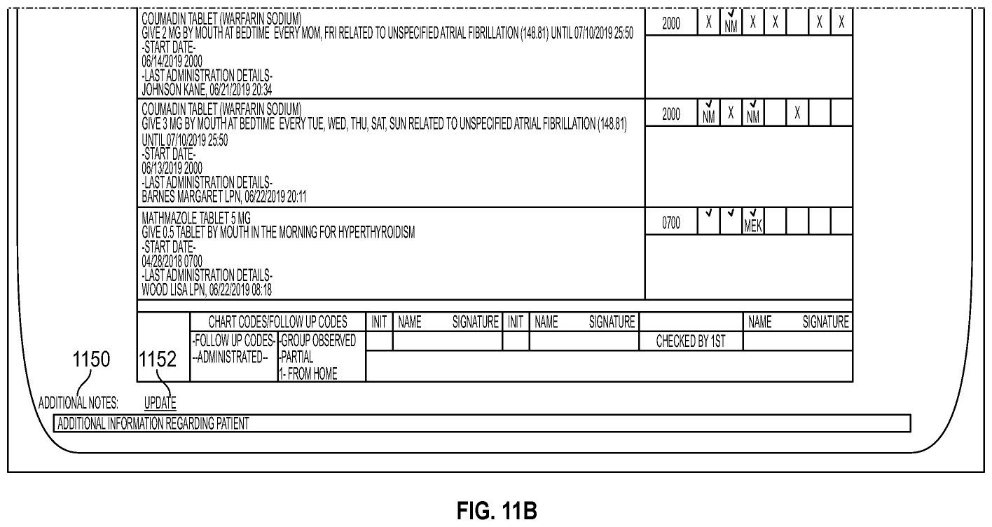

Displacement user interface 1000 includes one or more resident profiles 1002, one or more staff profiles 1004, one or more receiving facilities 1006 and upload 1040. Resident profiles 1002 includes all the residents associated with the LTC facility. In some embodiments, a user manually enters resident data (e.g., by interacting with upload 1040, etc.) into displacement user interface 1000. Additionally or alternatively, displacement user interface 1000 may automatically populate at least some of resident profiles 1002, staff profiles 1004, and/or receiving facilities 1006. For example, displacement user interface 1000 may retrieve a list of residents at an LTC facility from a server associated with the LTC facility and populate resident profiles 1002 based on the list. In some embodiments, upload 1040 causes automatic population of resident and staff data. Automatic population of resident and staff data is described in more detail below with reference to FIGS. 12-13. In some embodiments, a mix of automated and manual entry is used. For example, displacement user interface 1000 may automatically populate resident profiles 1002 but allow a user to manually review and/or edit the information. In various embodiments, displacement user interface 1000 is configured to facilitate users to customize displacement user interface 1000. For example, a user may choose how many residents are displayed on each page or may choose what resident information is displayed. In some embodiments, receiving facilities 1006 are one or more LTC facilities with which the displacing facility has a mutual occupancy agreement. Additionally or alternatively, receiving facilities 1006 may include other LTC facilities (e.g., LTC facilities within a vicinity of the displacing facility, a pool of LTC facilities, etc.).

Displacement user interface 1000 facilitates a user assigning residents and staff to a receiving facility 1006. For example, a user may drag a resident profile 1002 to a receiving facility 1006 to assign the resident to the receiving facility. In various embodiments, once the resident is assigned to the receiving facility, displacement user interface 1000 may send information associated with the resident (e.g., medical records, resident profile, etc.) to the receiving facility. In some embodiments, displacement user interface 1000 sends the receiving facility a notification indicating that resident has been assigned. In various embodiments, displacement user interface 1000 facilitates assigning and/or editing one or more existing assignments. For example, a user may remove a health care professional and a resident assigned to a particular receiving facility and reassign them to a different receiving facility.

Resident profiles 1002 includes resident picture 1010, description 1012, and expand option 1014. In some embodiments, resident picture 1010 is a headshot of the resident. In various embodiments, resident picture 1010 is retrieved from medical records associated with the resident. Additionally or alternatively, resident picture 1010 may be retrieved from a server and/or database associated with the LTC facility. For example, resident picture 1010 may be retrieved from a contacts list maintained by the LTC facility. In some embodiments, displacement user interface 1000 includes the ability to take a photo of the resident. Additionally or alternatively, resident picture 1010 may be received via a website. For example, a user may upload resident picture 1010 to a website and resident picture 1010 may be imported into resident profiles 1002. In some embodiments, selection of resident picture 1010 displays an enlarged version of resident picture 1010. Additionally or alternatively, selection of resident picture 1010 may facilitate editing (e.g., change, add filters, add tags, take a new picture, etc.) of resident picture 1010. In some embodiments, description 1012 includes the name, age, date of birth, and/or allergies of the resident. In some embodiments, description 1012 includes a different number and/or type of information. For example, description 1012 may include resident aliases (e.g., nicknames, etc.).

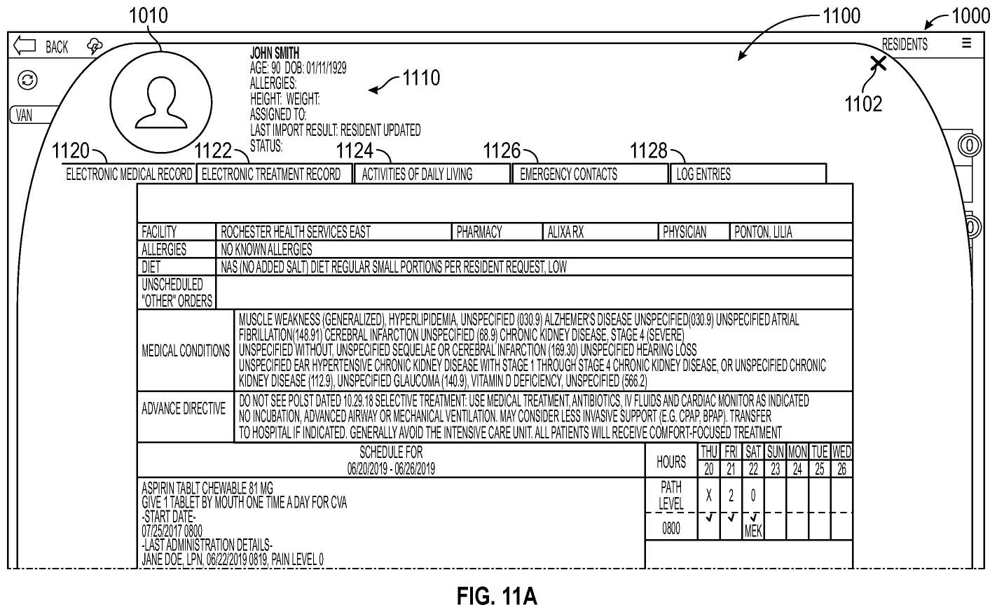

In some embodiments, expand option 1014 facilitates viewing additional details associated with resident profile 1002. For example, expand option 1014 may display medical records associated with the resident, as described in detail below with reference to FIGS. 11A-11B. In various embodiments, a user customizes what information is displayed by expand option 1014.

Staff profile 1004 includes staff picture 1020 and assign option 1022. Similar to resident picture 1010, in some embodiments, staff picture 1020 is a headshot of the staff member. In various embodiments, staff profile 1004 includes a different number and/or type of information associated with each staff member (e.g., specialties, certifications, etc.). In some embodiments, staff picture 1020 includes any of the features described above in reference to resident picture 1010. Assign option 1022 facilitates assigning a staff member to a receiving facility (e.g., receiving facility 1006, etc.). In some embodiments, when no staff are assigned to a receiving facility 1006, assign option 1022 exists independent of staff profile 1004. In some embodiments, assign option 1022 is a button (e.g., an addition operator symbol, etc.). Additionally or alternatively, assign option 1022 may be or include staff picture 1020. Additionally or alternatively, once assigned to a receiving facility, staff profile 1004 may indicate that the staff member is assigned (e.g., by changing an appearance of staff profile 1004, etc.). In some embodiments, a user selects one or more elements of staff profile 1004 to view additional information and/or edit the information associated with the staff member.

Receiving facility 1006 includes assign button 1030, availability 1032, and facility information 1034. Assign button 1030 is configured to facilitate assigning residents (e.g., resident profile 1002, etc.) and/or staff members (e.g., staff profile 1004, etc.) to a receiving facility (e.g., receiving facility 1006, etc.). In some embodiments, assign button 1030 is a button (e.g., an addition operator symbol, etc.). Additionally or alternatively, assign button 1030 may be or include receiving facility 1006. In some embodiments, assign button 1030 opens a dialog to select one or more resident profiles 1002 and/or staff profiles 1004 for assignment. In some embodiments, assigning one or more individuals updates availability 1032. In some embodiments, availability 1032 indicates the number of available beds remaining at the receiving facility. In various embodiments, availability 1032 is linked to receiving user interface 800. For example, availability 1032 may update based on changes in the number of available beds, as described in detail above with reference to FIGS. 8-9. Facility information 1034 includes information associated with receiving facility 1006. In some embodiments, facility information 1034 includes a facility name, address, contact information, website, and/or any other information associated with the receiving facility.

Turning now to FIGS. 11A-11B, details pane 1100 of displacement user interface 1000 is shown, according to an exemplary embodiment. In some embodiments, details pane 1100 displays in-depth information associated with a resident (e.g., resident profile 1002, etc.). In some embodiments, details pane 1100 is displayed in response to a user selecting expand option 1014. For example, details pane 1100 may display medical records associated with the resident. Details pane 1100 includes resident picture 1010, close button 1102, and one or more panes 1120-1128. Selection of close button 1102 closes details pane 1100. Additionally or alternatively, selection outside of details pane 1100 may close details pane 1100.

Panes 1120-1128 include organized information associated with a resident (e.g., resident profile 1002, etc.). In various embodiments, panes 1120-1128 are generated and/or populated based on information retrieved from a medical record associated with a resident. Panes 1120-1128 include electronic medical records 1120, electronic treatment records 1122, activities of daily living 1124, emergency contacts 1126, and log entries 1128. In some embodiments, panes 1120-1128 include a different number and/or type of panes. In various embodiments, a user may customize the appearance and contents of panes 1120-1128. In some embodiments, electronic medical records 1120 include additional notes 1150. Electronic medical records 1120 displays one or more medical records associated with the resident. In some embodiments, electronic medical records 1120 display live, up to date medical records. Additionally or alternatively, electronic medical records 1120 may display a backed up copy of the medical records.

Additional notes 1150 include update option 1152. In some embodiments, update option 1152 opens a dialog to update (e.g., edit, modify, alter, add, remove, etc.) information from one or more panes (e.g., panes 1120-1128, etc.) of details pane 1100. For example, a user may add a note about a resident's favorite activities to additional notes 1150.

Electronic treatment records 1122 includes one or more electronic treatment records. In some embodiments, electronic treatment records 1122 include a list of recent treatments a resident has received. For example, electronic treatment records 1122 may include a chart indicating when a patient has received their daily medications.

Activities of daily living 1124 includes one or more activities associated with the resident. In some embodiments, activities of daily living 1124 include exercises and/or physical therapy routines for the resident. In some embodiments, activities of daily living 1124 indicate a number of self-care routines of a resident. For example, activities of daily living 1124 may indicate that resident brushes their own teeth daily. Additionally or alternatively, activities of daily living 1124 may indicate which activities a resident needs help completing. For example, activities of daily living 1124 may indicate a resident requires help to bathe himself or herself.

Emergency contacts 1126 includes one or more emergency contacts. In some embodiments, emergency contacts 1126 includes phone numbers, email addresses, postal addresses, and/or other contact information. In some embodiments, emergency contacts 1126 includes one or more pictures and/or name pronunciations associated with the listed emergency contacts. Log entries 1128 includes one or more record and/or log entries. In some embodiments, log entries 1128 includes a history of recent updates to resident profile 1002. For example, log entries 1128 may indicate the last time the information associated with the resident was retrieved from a master electronic medical record. In some embodiments, log entries 1128 includes one or more notes taken by a health care professional. For example, log entries 1128 may include a note indicating the resident felt ill after consuming dairy.

Referring now generally to FIGS. 12-14, a number of flow diagrams are shown, according to an exemplary embodiment. In some embodiments, FIGS. 12-14 represent one or more backend systems of the resident displacement manager (e.g., resident displacement manager 664, receiving user interface 800, displacement user interface 1000, etc.) of the present disclosure. For example, the backend systems may be one or more computing devices, circuits, servers, databases, microprocessors, routines, threads, virtual machines, containers, or the like. Additionally or alternatively, one or more devices (e.g., a mobile response unit, etc.) may act in coordination with the backend systems.

As shown in FIG. 12, a method 1200 of set-up for resident displacement manager 664 is performed during a configuration (e.g., set-up, etc.) and/or installation process for resident displacement manager 664. Additionally or alternatively, method 1200 may be performed to import and/or update data. For example, a user, via method 1200, may import medical records into resident displacement manager 664 to automatically populate displacement user interface 1000. In other embodiments, resident displacement manager 664 omits method 1200. In some embodiments, method 1200 occurs automatically in response to an event (e.g., a stale data server response, etc.). In some embodiments, method 1200 is triggered by a user (e.g., via upload 1040, etc.). In various embodiments, method 1200 facilitates customizing (e.g., adding/removing modules, changing a layout, changing a look and feel, etc.) resident displacement manager 664.

At step 1210, a user interface (e.g., receiving user interface 800, displacement user interface 1000, etc.) of resident displacement manager 664 receives a set-up request. In various embodiments, a user selects a configuration menu option to perform step 1210. At step 1220, a user interface of resident displacement manager 664 receives a data location. In some embodiments, the data location is a server and/or database address (e.g., an IP address, a MAC address, etc.). Additionally or alternatively, the data location may be a file path and/or a link (e.g., a HTML link, etc.) to a file or other electronic medium having the data. In some embodiments, step 1220 includes pinging (e.g., issuing a packet internet groper (“PING”) command, etc.) the location indicated by the data location to verify that it is valid.

At step 1230, a user interface of the resident displacement manager 664 receives a data type. The data type may indicate a source of the data. For example, the data type may indicate the data location is a server or a cloud based document. Additionally or alternatively, the data type may indicate a category of data. For example, the data type may indicate the data at the data location is medical records. In some embodiments, method 1200 handles the data differently depending on the data type. For example, a first “contacts” data type may be parsed to extract contacts for a LTC facility contacts list, while a second “medical records” data type may be linked to resident profiles in displacement user interface 1000.

At step 1240, a user interface of the resident displacement manager 664 imports the data specified by the data location. In various embodiments, step 1240 varies based on the data type of the data being imported. For example, “medical records” data may be stored locally on a device (e.g., a mobile response unit, etc.) and also redundantly uploaded to a cloud server, while “contacts” data may be only saved locally. In some embodiments, importing the data includes populating one or more user interfaces (e.g., receiving user interface 800, displacement user interface 1000, etc.). For example, step 1240 may include generating a number of resident profiles 1002 based on resident data from a LTC facility. In some embodiments, step 1240 modifies already existing data and/or combines multiple data sources. For example, step 1240 may include generating a number of resident profiles 1002 based on a combination of resident data and medical records (e.g., EMR, etc.) data. In various embodiments, method 1200 is used to import data of any kind. For example, method 1200 may import contacts into a contacts module (e.g., emergency contacts 650, etc.) of user interface 600.

As shown in FIG. 13, a method 1300 of backup is performed automatically (e.g., on a schedule, in response to a stale data server response, etc.) to refresh data and/or maintain up to date records. Although method 1300 is described in terms of backing up data from an external server, it should be understood that method 1300 also applies to backing up local data. In some embodiments, method 1300 is data specific. For example, medical records may be backed up hourly while contacts may be backed up monthly. In some embodiments, method 1300 is performed at least partially in associated with method 1200. For example, method 1300 may be understood to be part of step 1240 of method 1200.

At step 1310, data is retrieved from an external server. In some embodiments, the external server is a third party server. For example, the external server may be an electronic medical record provider server. In some embodiments, the external server can be a different data source (e.g., a database, a spreadsheet, etc.). In some embodiments, step 1310 includes a GET request and/or any HTTP request to the external server. In some embodiments, the method of retrieval depends on the data source (e.g., external server, etc.) type. For example, first data may be retrieved from a first server using a GET request while second data from a second server may be retrieved via a publish/subscribe model. Accordingly, in some embodiments, a data push initiates method 1300 instead of an active retrieval (e.g., a GET request, etc.). In some embodiments, step 1310 includes parsing the retrieved data. For example, the retrieved data may be an unordered list of contacts, and step 1310 may include alphabetizing and/or categorizing the list. In some embodiments, step 1310 includes comparing the data at the external server to existing data to determine what data has changed. For example, step 1310 may include only retrieving new and/or changed data.

At step 1320, the data is stored locally. In various embodiments, step 1320 includes storing the data in memory. In some embodiments, step 1320 is omitted and/or the data is only stored in the cloud (step 1330). Additionally or alternatively, step 1320 may include populating one or more user interfaces (e.g., resident displacement manager 664, receiving user interface 800, displacement user interface 1000, etc.) with the data. For example, step 1320 may include populating resident profiles 1002. In some embodiments, step 1320 includes notifying a user of the update. In some embodiments, step 1320 includes an indication of what data has changed and/or is new. For example, an appearance of newly imported resident profiles 1002 may be changed.

At step 1330, the data is stored in the cloud. In various embodiments, step 1330 includes storing the data in memory of one or more remotely hosted computing devices, circuits, servers, databases, a combination thereof, or the like. In some embodiments, the cloud is a computing device maintained by the LTC facility. In some embodiments, step 1330 includes a RESTful API (e.g., PUT request, POST request, etc.) to interact with the cloud. Additionally or alternatively, step 1330 may include a publish/subscribe model. In some embodiments, step 1330 may include writing to an audit log to document backup of the data.

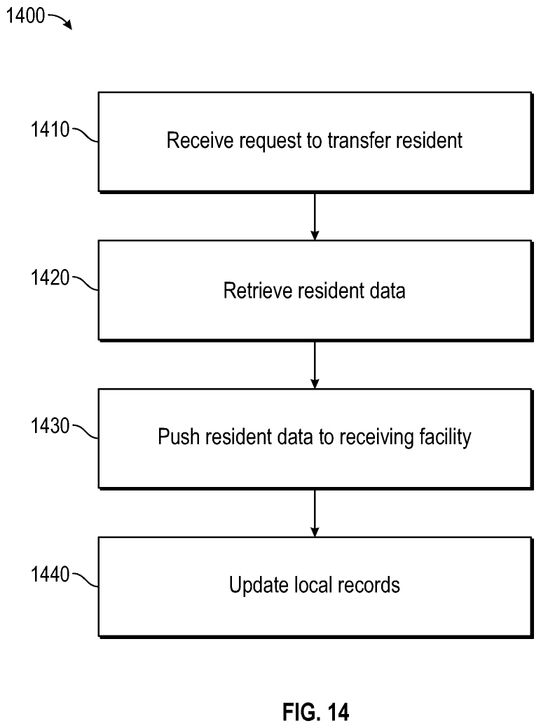

Turning now to FIG. 14, a flow diagram of method 1400 for transferring residents is shown, according to an exemplary embodiment. In some embodiments, method 1400 is performed in response to a user assigning a resident to a receiving facility, as described in detail above with reference to FIGS. 10-11B. At step 1410, a request is received to transfer a resident. In some embodiments, step 1410 includes a determination of priority (e.g., time, location, need, etc.). In some embodiments, the priority is user customizable. For example, a user may create a priority rule to reflect a term included in a mutual occupancy agreement between a displacing facility and a receiving facility. For further example, a first request for transfer from a first displacing facility located next to a severe weather event may have priority over a second request for transfer from a second displacing facility located remotely of a severe weather event. In various embodiments, the request includes information associated with the resident and the transfer (e.g., a resident ID, resident medical records, a link to resident information, an IP address of a server associated with the receiving facility, etc.).

At step 1420, resident data is retrieved. In some embodiments, step 1420 includes retrieving (e.g., downloading, acquiring a link to, etc.) resident data from a server or other location. In some embodiments, resident data is stored locally (e.g., on a mobile response unit, etc.) and is packaged for distribution. Additionally or alternatively, step 1420 may include any of the data retrieval steps described above in reference to FIGS. 12-13. In some embodiments, step 1402 includes generating and/or obtaining a pointer to the data location (e.g., an IP address of the server, etc.). In some embodiments, one or more authentication methods (e.g., passwords, keys, etc.) are included in the pointer to allow access to the data.

At step 1430, the data is pushed to the receiving facility. In some embodiments, pushing the data to the receiving facility includes sending the data to a server associated with the receiving facility. In some embodiments, step 1430 includes notifying a user associated with the receiving facility of the data. In some embodiments, step 1430 includes sending an email with the data to an email address associated with the receiving facility. Additionally or alternatively, step 1430 may include providing the receiving facility access to the data through a user interface (e.g., receiving user interface 800, etc.). In some embodiments, the data is provided to the receiving facility by sending a pointer to the data to the receiving facility. In some embodiments, step 1430 includes a RESTful API (e.g., PUT request, POST request, etc.) to interact with the receiving facility.

At step 1440, local records are updated. In some embodiments, updating local records includes updating one or more records associated with a displacing facility (e.g., a resident management server of a displacing facility, etc.). In some embodiments, step 1440 includes writing to an audit record to indicate transfer of the residents. Additionally or alternatively, device (e.g., a mobile response unit, etc.) memory may be updated to reflect the change. In some embodiments, step 1440 includes triggering a printer to print a transfer receipt. Additionally or alternatively, step 1440 may include sending one or more notifications (e.g., text messages, emails, automated phone calls, etc.) to notify one or more individuals of the transfer. For example, step 1440 may include an automated call to one or more family members of the transferred resident. Additionally or alternatively, step 1440 may include updating one or more user interfaces (e.g., resident displacement manager 664, receiving user interface 800, displacement user interface 1000, etc.). For example, availability 1032 may be updated.

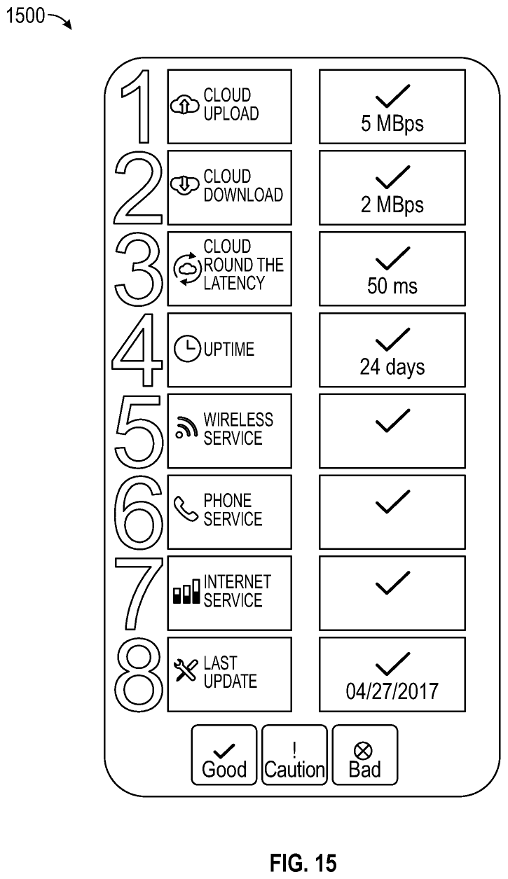

Referring now to FIG. 15, an illustrative GUI that may be used within a system for emergency preparedness (e.g., mobile response unit 100, resident displacement manager 664, etc.) is shown, according to some embodiments of the present disclosure. In some embodiments, the system may monitor, on a continuous or near-continuous basis, all facility devices (e.g., user device 202, etc.) within the system. The illustrative GUI 1500 may provide a health check report for a particular facility device. GUI 1500 may be accessible by admins and/or members of the first response team (e.g., using respective admin devices and/or user device 202). In some embodiments, GUI 1500 may correspond to a health check report that is sent to a facility on a periodic basis (e.g., monthly). The illustrative GUI 1500 may include several facility-related metrics, such as the status of the facility's phone and wireless service, patch information, upload time, and information regarding any outages at the facility.

### Disaster Relief System

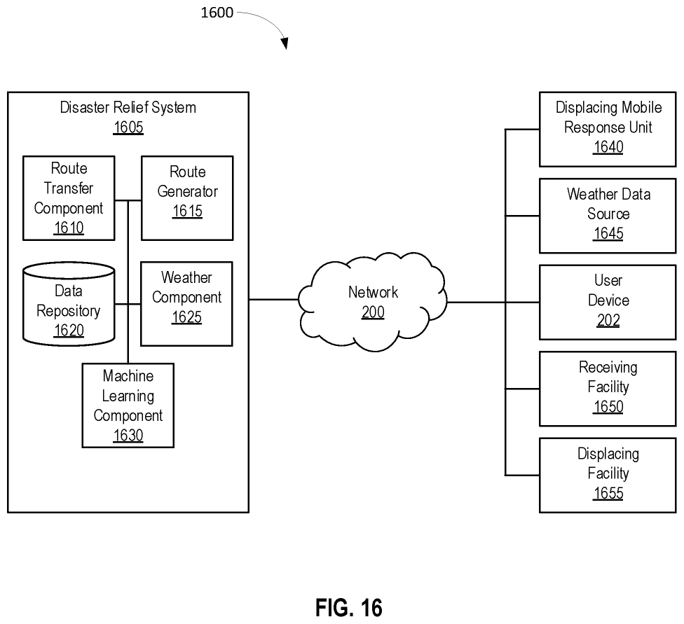

Referring now to FIG. 16 a block diagram of a system 1600 is shown, according to an exemplary embodiment. The system 1600 can include at least one disaster relief system 1605, at least one displacing mobile response unit 1640, at least one weather data source 1645, at least one user device 202, at least one receiving facility 1650, at least one displacing facility 1655, and the network 200. The disaster relief system 1605 can include at least one route transfer component 1610, at least one route generator 1615, at least on data repository 1620, at least one weather component 1625 and at least one machine learning component 1630. In some embodiments, at least one component of the disaster relief system 1605 includes similar components to that of the mobile response unit 100. In some embodiments, the disaster relief system 1605 can perform similar functionality to that of the mobile response unit 100. In some embodiments, at least one component of the displacing mobile response unit 1640 includes similar components to that of the mobile response unit 100. In some embodiments, the displacing mobile response unit 1640 can perform similar functionality to that of the mobile response unit 100. In some embodiments, the mobile response unit 100 can perform similar functionality to that of the displacing mobile response unit 1640. In some embodiments, the displacing mobile response unit 1640 can be associated with the displacing facility 1655. In some embodiments, the mobile response unit 100 and/or the displacing mobile response unit 1640 can perform similar functionality to that of the disaster relief system 1605. In some embodiments, at least one component of the mobile response unit 100 or at least one component of the displacing mobile response unit 1640 includes similar components to that of the disaster relief system 1605. In some embodiments, the receiving facility 1650 can be at least one of the receiving facilities 1006.

The route transfer component 1610 can interface, interact or communicate, via the network 200, with at least one of the displacing mobile response unit 1640, the user device 202, the receiving facility 1650, the displacing facility 1655 and/or an external system. The route transfer component 1610 can receive a request for directions to a receiving facility (e.g., direction to the receiving facility 1650). In some embodiments, the route transfer component 1610 can receive the request from at least one of the displacing mobile response unit 1640, a user device associated with the displacing facility 1655 (e.g., the user device 202), the displacing facility 1655 and/or an external system (e.g., a remote center). For example, the route transfer component 1610 can receive, from the displacing mobile response unit 1640, the request for directions to the receiving facility 1650. In some embodiments, the request can include a location of a resident associated with the displacing facility 1655 and a location of a caretaker that is associated with the resident. In some embodiments, the location of the resident and the location of the caretaker can be the same location (e.g., the location of the displacing facility 1655). In some embodiments, the location of the resident and the location of the caretaker are not the same location. In some embodiments, the location of the resident can be the location of the displacing mobile response unit 1640. In some embodiments, the request can include a location of a receiving facility (e.g., a location of the receiving facility 1650). In some embodiments, the location of the receiving facility 1650 and/or the location of the displacing facility 1655 are stored, kept, maintained, or otherwise located in the data repository 1620. In some embodiments, the route transfer component 1610 can receive, responsive to receiving the request for directions to the receiving facility 1650, a location, from the data repository 1620, of the displacing facility 1655 and/or the receiving facility 1650. The route transfer component 1610 can provide, to the route generator 1615, the location of the resident and the location of the receiving facility 1650.

The route generator 1615 can generate, determine, identify, or otherwise create at least one route based on the location of the resident and the receiving facility 1650. For example, the route generator 1615 can identify at least one of a roadway, a street, an interstate, a highway, a freeway and/or an expressway that can be taken, traveled, or otherwise followed in order to take the resident from their current location to the receiving facility 1650. In some embodiments, the route generated by the route generator 1615 can include various combinations of roadways, streets, interstates, highways, freeways and/or expressways. For example, the route can begin by traveling on a street that leads away from the location of the resident, the street can lead to an interstate, the interstate can lead to a subsequent street and the subsequent street can lead to the receiving facility 1650. The route generator 1615 can provide, to the route transfer component 1610, the route.

The route transfer component 1610, responsive to receiving the route from the route generator 1615, can provide, to the displacing mobile response unit 1640, a route to the receiving facility 1650. In some embodiments, the route transfer component 1610 can provide, to the user device 202 that is associated with the displacing facility 1655, the route to the receiving facility 1650. The route transfer component 1610, responsive to providing the route to the displacing mobile response unit 1640, can provide an indication to the receiving facility 1650. The indication can include a notification that the resident and/or a caretaker associated with the resident are in route to the receiving facility 1650. In some embodiments, the indication can be provided to at least one of a user device (e.g., the user device 202) associated with the receiving facility 1650, a mobile response unit 100 associated with the receiving facility 1650 and/or a notification system associated with the receiving facility 1650 (e.g., a display screen located near a wing of the receiving facility 1650 that can receive residents). In some embodiments, the indication can include at least one record associated with the resident and/or at least one record associated with the caretaker. For example, the indication can include a medical record that pertains to the resident. The indication can also include a record that provides information pertaining to the caretaker. For example, the record can include all of the caretaker's qualifications and/or responsibilities.

In some embodiments, the disaster relief system 1605 can receive a request, responsive to providing the indication to the receiving facility 1650, to provide at least one record that is associated with the resident and/or caretaker associated with the resident. In some embodiments, the displacing facility 1655 can provide the records to the receiving facility 1650. For example, the records can be stored locally on the displacing mobile response unit 1640 and an operator of the displacing mobile response unit 1640 can provide the records to the receiving facility 1650.

The weather component 1625 can interface, interact, or otherwise communicate, via the network 200, with the weather data source 1645. The weather data source 1645 can include at least one of a government weather system (e.g., NOAA, National Weather service, etc.) and/or a third-party weather system (e.g., WeatherBug, AccuWeather, a weather application programming interface (API) or any other possible third-party weather system). The weather component 1625 can receive, from the weather data source 1645, a weather alert. In some embodiments, the weather alert can include information that pertains to a weather event. For example, the weather alert can include information about a hurricane. In some embodiments, the information can include at least one of a location of the weather event, a predicted travel pattern of the weather event, a predicted duration of the weather event and/or a predicted severity of the weather event. The weather component 1625, responsive to receiving the weather alert, can determine that the route, provided to the displacing mobile response unit 1640, will be impacted by the weather event.

In some embodiments, the route will be impacted by the weather event if a road closure occurs (e.g., a portion of the route is or will be closed due to the weather event). In some embodiments, the weather component 1625 can interface, interact, or otherwise communicate with the route generator 1615. The weather component 1625 can provide, to the route generator 1615, an indication that the route provided to the displacing mobile response unit 1640 will be impacted by the weather event. In some embodiments, the route generator 1615, responsive to communicating with the weather component 1625, can update the route provided to the displacing mobile response unit 1640. In some embodiments, the disaster relief system 1605 and/or the displacing mobile response unit 1640 can perform multicasting. In some embodiments the route from the displacing facility 1655 to the receiving facility 1650 can be displayed one or more devices (e.g., multicast). In some embodiments, the indication that the resident and the caretaker associated with the resident are in route to the receiving facility 1650 can be displayed by one or more devices. In some embodiments, an alert that a weather event will impact the route can be provided by multicast. In some embodiments, an alert that a weather event will impact the receiving facility 1650 can be provided by multicast. For example, the displacing mobile response unit 1640 can perform multicast by displaying, via a display (e.g. the display 310), the route from the displacing facility 1655 to the receiving facility 1650 and by causing a device (e.g., the user device 202) to also display the route from the displacing facility 1655 to the receiving facility 1650.

The route generator 1615 can provide, to the route transfer component 1610, the updated route. The route transfer component 1610 can provide the updated route to the displacing mobile response unit 1640. In some embodiments, the route transfer component 1610 can provide the updated route to the user device 202 that is associated with the displacing facility 1655. In some embodiments, the route is updated to avoid the weather event. For example, the route generator 1615 can identify at least one of a roadway, a street, an interstate, a highway, a freeway and/or an expressway that can be taken to avoid the weather event. In some embodiments, the route transfer component 1610, responsive to the route being updated, can provide an alert, to the displacing mobile response unit 1640, an alert that the route has been updated. The route transfer component 1610 can provide, to the user device associated with the displacing facility 1655, the alert. In some embodiments, the alert can cause the displacing mobile response unit 1640 to display, via a user interface (e.g., the user interface 120), a prompt to accept the updated route. The route transfer component 1610 can receive, from the displacing mobile response unit 1640, an indication that an operator of the displacing mobile response unit 1640 has selected the prompt to accept the updated route.

The machine learning component 1630 can monitor, facilitate, track, or otherwise assist in the transfer of a resident from a displacing facility to a receiving facility. For example, the machine learning component 1630 can identify at least one resident that can be transferred from a displacing facility (e.g., the displacing facility 1655) to a receiving facility (e.g., the receiving facility 1650 and/or at least one of the receiving facilities 1006). The machine learning component 1630 can include at least one of supervised machine learning, unsupervised machine learning, neural networks, decision trees, Bayesian networks and/or hierarchical Bayesian networks. In some embodiments, the machine learning component 1630 can use information, provided by the weather component 1625, to determine that a facility will be impacted by a weather event. For example, the machine learning component 1630 can determine that the displacing facility 1655 will be impacted by a hurricane. The machine learning component 1630, responsive to determining that the displacing facility 1655 will be impacted by a weather event, can identify at least one facility (e.g., receiving facility 1650) that can receive at least one resident of the displacing facility 1655. In some embodiments, the machine learning component 1630 can use the weather event to identify the receiving facility 1650. For example, the machine learning component 1630 can identify the receiving facility 1650 by determining that the receiving facility 1650 is not predicted to be impacted by the weather event.

The machine learning component 1630, responsive to identifying the receiving facility 1650, can interface, interact or otherwise communicate with the data repository 1620 to receive information that pertains to at least one resident of the displacing facility 1655 and/or information that pertains to the receiving facility 1650. The machine learning component 1630 can use the information, provided by the data repository 1620, to identify at least one resident that can be transferred from the displacing facility 1655 to the receiving facility 1650. For example, the machine learning component 1630 can receive, from the data repository 1620, a medical record that is associated with a resident. The machine learning component 1630 can, using the medical record, determine that the receiving facility 1650 can receive the resident. In some embodiments, the machine learning component 1630, responsive to determining that the receiving facility 1650 can receive the resident, can provide an indication to the route generator 1615. The indication can include the location of the receiving facility 1650 and the location of the resident. In some embodiments, the machine learning component 1630 can provide the indication to the displacing mobile response unit 1640 and/or the user device 202 that is associated with the displacing facility 1655.

The route generator 1615, responsive to receiving the indication from the machine learning component 1630, can generate a route to the receiving facility 1650. The route generator 1615 can provide, to the route transfer component 1610, the route. The route transfer component 1610 can, responsive to receiving the route from the route generator 1615, provide, to at least one of the displacing mobile response unit 1640 and/or the user device 202 that is associated with the displacing facility 1655, the route.

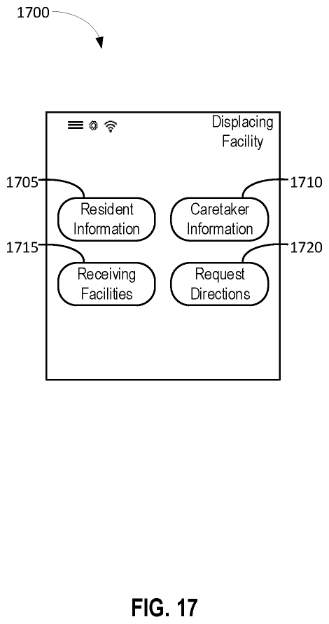

Referring now to FIG. 17, an example user interface 1700 is shown. The user interface 1700 can be generated, provided, presented, shown, or otherwise displayed by the displacing mobile response unit 1640. In some embodiments, the user interface 1700 can be displayed by a user device (e.g., the user device 202) that is associated with the displacing facility 1655. In some embodiments, the user interface 1700 can display information that was provided by the disaster relief system 1605. For example, the user interface 1700 can display the route that was provided by the route transfer component 1610. The user interface 1700 can include a resident information icon 1705, a caretaker information icon 1710, a receiving facilities icon 1715 and a request directions icon 1720.

An operator of the displacing mobile response unit 1640 can select the resident information icon 1705 to view or otherwise be provided information that pertains to residents of a displacing facility (e.g., the displacing facility 1655). In some embodiments, the information can include a list of the residents that currently reside within the displacing facility 1655. In some embodiments, the operator of the displacing mobile response unit 1640 can select a resident from the list of resident to view more information about the selected resident. In some embodiments, the operator of displacing mobile response unit 1640 can be directed, responsive to selecting the resident, to a user interface that is similar to that shown in FIG. 11A. The operator of the displacing mobile response unit 1640 can select the caretaker information icon 1710 to view information that pertains to caretakers of the displacing facility 1655. For example, the information can include a list of the caretakers as well as a list of residents that each caretaker provides care too.

The operator of the displacing mobile response unit 1640 can select the receiving facilities icon 1715 to view information that pertains to receiving facilities. For example, the information can include a list of facilities that can receive at least one resident from the displacing facility 1655. The operator of the displacing mobile response unit 1640 can select the request directions icon 1720 to provide a request, to the disaster relief system 1605, for directions to a receiving facility that the operator had chosen. In some embodiments, the operator can be prompted, responsive to selecting the request directions icon 1720, to provide the location of the resident and/or the location of the receiving facility.

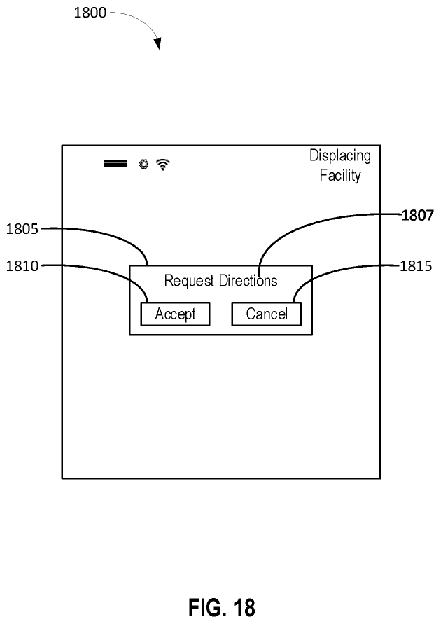

Referring now to FIG. 18, a user interface 1800 is shown. The user interface 1800 can be generated, provided, presented, shown, or otherwise displayed by the displacing mobile response unit 1640. In some embodiments, the user interface 1800 can be displayed by the user device that is associated with the displacing facility 1655. In some embodiments, the user interface 1800 can be generated responsive to the operator selecting the request directions icon 1720. In some embodiments, the user interface 1800 can be an overlay on top of the user interface 1700. In some embodiments, the user interface 1800 is a separate user interface. The user interface 1800 can display similar information to that of user interface 1700. In some embodiments, the user interface 1700 and the user interface 1800 can be combined into a single user interface.

The user interface 1800 can include a window 1805. The window 1805 can be or include at least one of a dialog box, a dialog window, a display window, a display area, or a prompt including an indication. The window 1805 can include a request directions prompt 1807, an accept icon 1810 and a cancel icon 1815. The operator can select the accept icon 1810 to complete the request for directions to the receiving facility. The operator can select the cancel icon 1815 to cancel the request for directions to the receiving facility. In embodiments, the disaster relief system 1605 can receive, responsive to the operator selecting the accept icon 1810, the request for directions to the receiving facility.

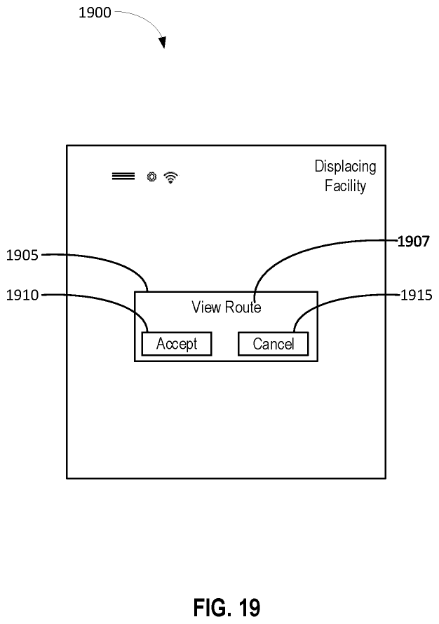

Referring now to FIG. 19, a user interface 1900 is shown. The user interface 1900 can be generated, provided, presented, shown, or otherwise displayed by the displacing mobile response unit 1640. In some embodiments, the user interface 1900 can be displayed by the user device that is associated with the displacing facility 1655. In some embodiments, the user interface 1900 can be generated responsive to the operator selecting the accept icon 1810. In some embodiments, the user interface 1900 is generated responsive to the disaster relief system 1605 providing, to at least one of the displacing mobile response unit 1640 and/or the user device associated with the displacing facility 1655, the route to the receiving facility 1650. In some embodiments, the user interface 1900 can be an overlay of the user interface 1700 and/or the user interface 1800. In some embodiments, the user interface 1900 is a separate user interface. The user interface 1900 can display similar information to that of the user interface 1700 and/or the user interface 1800. In some embodiments, the user interface 1900, the user interface 1800 and/or the user interface 1700 can be combined into a single user interface.

The user interface 1900 can include a window 1905. The window 1905 can be or include at least one of a dialog box, a dialog window, a display window, a display area, or a prompt including an indication. The window 1905 can include a view route prompt 1907, an accept icon 1910 and a cancel icon 1915. The operator can view, responsive to selecting the accept icon 1910, the route to the receiving facility 1650. The operator can select the cancel icon 1915 to remove the window 1905. In some embodiments, selecting the cancel icon 1915 can cancel the request to receive directions to the receiving facility 1650.

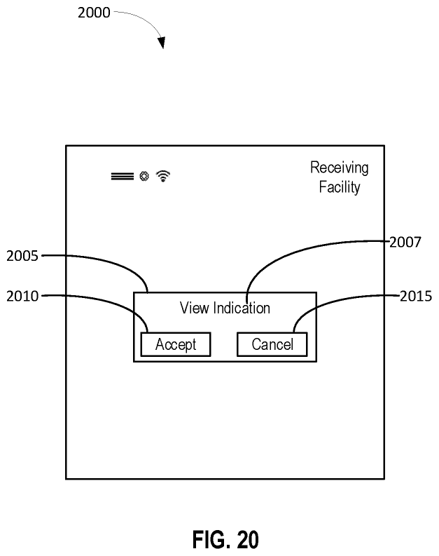

Referring now to FIG. 20, a user interface 2000 is shown. The user interface 2000 can be generated, provided, presented, shown, or otherwise displayed by at least one of a mobile response unit (e.g., the mobile response unit 100) that is associated with the receiving facility 1650 and/or a user device (e.g., the user device 202) that is associated with the receiving facility 1650. In some embodiments, the user interface 2000 can display information that was provided by the disaster relief system 1605. In some embodiments, the user interface 2000 can be generated responsive to the operator of the displacing mobile response unit 1640 selecting at least one of the request directions icon 1720, the accept icon 1810 and/or the accept icon 1910. In some embodiments, the user interface 2000 is generated responsive to the disaster relief system 1605 providing the indication, to at least one of the mobile response unit associated with the receiving facility 1650 and/or the user device associated with the receiving facility 1650, that the resident and the caretaker associated with the resident are in route to the receiving facility 1650.

The user interface 2000 can include a window 2005. The window 2005 can be or include at least one of a dialog box, a dialog window, a display window, a display area, or a prompt including an indication. The window 2005 can include a view indication prompt 2007, an accept icon 2010 and a cancel icon 2015. An operator of the mobile response unit associated with the receiving facility 1650 and/or an operator of the user device associated with the receiving facility 1650 can select the accept icon 2010 in order to view the indication that was provided by the disaster relief system 1605. The operator of the mobile response unit and/or the operator of the user device can select the cancel icon 2015 to remove the window 2005.

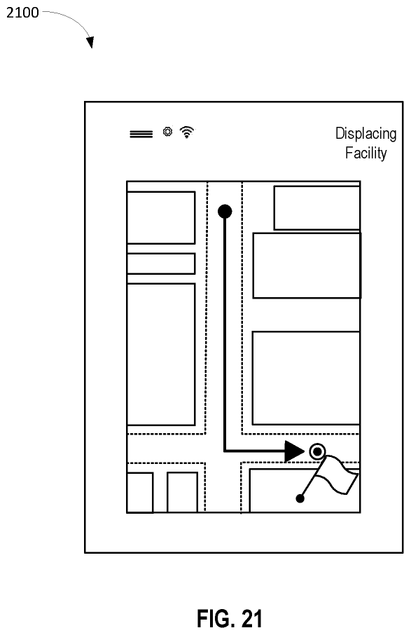

Referring now to FIG. 21, an example user interface 2100 is shown. The user interface 2100 can be generated, provided, presented, shown, or otherwise displayed by at least one the displacing mobile response unit 1640, the user device associated with the displacing facility 1655, the mobile response unit associated with the receiving facility 1650 and/or the user device associated with the receiving facility 1650. In some embodiments, the user interface 2100 can display information that was provided by the disaster relief system 1605. For example, the user interface 2100 can display the route to the receiving facility 1650. The user interface 2100 can be generated responsive to the disaster relief system 1605 providing, to the displacing mobile response unit 1640, the route to the receiving facility 1650. In some embodiments, the user interface 2100 can be generated responsive to the disaster relief system 1605 providing, to the mobile response unit associated with the receiving facility 1650, the indication that the resident and the caretaker associated with the resident are in route to the receiving facility 1650. In some embodiments, the user interface 2100 can provide step-by-step directions, to the operator of the displacing mobile response unit 1640, to the receiving facility. In some embodiments, the user interface 2100 can provide updates, to the operator of the mobile response unit associated with the receiving facility 1650, pertaining to the location of the resident and the caretaker associated with the resident. In some embodiments, the user interface 2100 can include at least one of a location of an additional mobile response unit (e.g., a second mobile response unit 100), a location of a government facility (e.g., a military base, a temporary hospital, etc.) and/or a location to an additional receiving facility (e.g., a second receiving facility 1650).

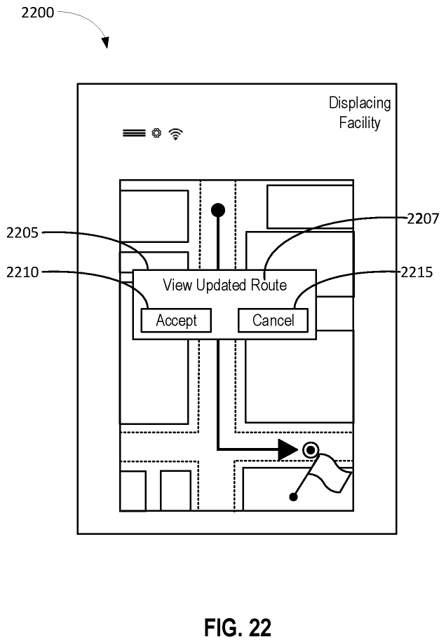

Referring now to FIG. 22, a user interface 2200 is shown. The user interface 2200 can be generated, provided, presented, shown, or otherwise displayed by at least one the displacing mobile response unit 1640, the user device associated with the displacing facility 1655, the mobile response unit associated with the receiving facility 1650 and/or the user device associated with the receiving facility 1650. In some embodiments, the user interface 2200 can display information that was provided by the disaster relief system 1605. The user interface 2200 can be an overlay on top of the user interface 2100. In some embodiments, the user interface 2200 is a separate user interface. The user interface 2200 can display similar information to that of user interface 2100. In some embodiments, the user interface 2200 and the user interface 2100 can be combined into a single user interface. In some embodiments, the user interface 2200 can be generated responsive to the disaster relief system 1605 and/or the displacing mobile response unit 1640 determining that the route to the receiving facility 1650 will be impacted by a weather event.

The user interface 2200 can include a window 2205. The window 2205 can be or include at least one of a dialog box, a dialog window, a display window, a display area, or a prompt including an indication. The window 2205 can include a view updated route prompt 2207, an accept icon 2210 and a cancel icon 2215. An operator of the displacing mobile response unit 1640 can select the accept icon 2210 to view the updated route that was provided by the disaster relief system 1605. The operator of the displacing mobile response unit 1640 can select the cancel icon 2215 to remove the window 2205.

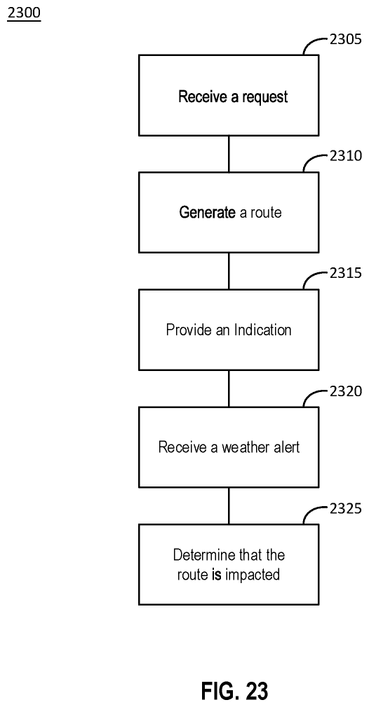

Referring now to FIG. 23, a flow diagram of a method 2300 for providing directions from a displacing facility to a receiving facility during a weather event is shown, according to an exemplary embodiment. In some embodiments, at least one step of the method 2300 can be performed by at least one of the disaster relief system 1605, the displacing mobile response unit 1640, the mobile response unit 100 and/or a component of the system 1600. In some embodiments, a facility (e.g., the displacing facility 1655) can be impacted by a weather event. For example, the displacing facility 1655 can be impacted by a hurricane. The weather event can result in at least one resident being displaced from the displacing facility 1655. The method 2300 described herein can facilitate a seamless transfer of a resident from the displacing facility 1655 to a receiving facility (e.g., the receiving facility 1650).

In step 2305, the route transfer component 1610 can receive a request for directions to the receiving facility 1650. In some embodiments, the route transfer component 1610 can receive the request from at least one of the displacing mobile response unit 1640, the user device associated with the displacing facility 1655 and/or an external system. The request can include a location of a resident and a location of a caretaker that is associated with the resident. The resident can currently reside within the displacing facility 1655. In some embodiments, the route transfer component 1610 can receive the request responsive to an operator of the displacing mobile response unit 1640 selecting the request directions icon 1720. In some embodiments, the route transfer component 1610 can receive, from the data repository 1620, the location of the displacing facility 1655 and/or the location of the receiving facility 1650. In some embodiments, the location of the displacing facility 1655 and/or the location of the receiving facility 1650 is provided with the request. The route transfer component 1610 can provide, to the route generator 1615, the location of the receiving facility 1650 and the location of the displacing facility 1655.

In step 2310, at least one route can be generated. For example, the route generator 1615 can, responsive to receiving the location of the displacing facility 1655 and the location of the receiving facility 1650, can generate a route, from the displacing facility 1655, to the receiving facility 1650. The route generator 1615 can provide, to the route transfer component 1610, the route to the receiving facility 1650. The route transfer component 1610 can, responsive to receiving the route from the route generator 1615, provide, to the displacing mobile response unit 1640, the route. In some embodiments, the route transfer component 1610 can, responsive to providing the route to the displacing mobile response unit 1640, can cause the displacing mobile response unit 1640 to display at least one of the user interface 1900 and/or the user interface 2100. In some embodiments, the route transfer component 1610 can cause the displacing mobile response unit 1640 to display the route.

In step 2315, the route transfer component 1610 can provide, to the receiving facility 1650, an indication that the resident and the caretaker associated with the resident are in route to the receiving facility 1650. In some embodiments, the route transfer component 1610 provides the indication responsive to an operator of the displacing mobile response unit 1640 selecting the accept icon 1910.

In step 2320, the weather component 1625 can receive, from the weather data source 1645, at least one weather alert. The weather alert can include information that pertains to at least one weather event. In some embodiments, the weather component 1625 can provide, to the weather data source 1645, a request for the weather alert. In some embodiments, the weather component 1625 can receive the weather alert continuously from the weather data source 1645. In some embodiments, the weather component 1625 can receive the weather alert in predetermined intervals. For example, the weather component 1625 can receive the weather alert every five minutes, 10 minutes, or any other time amount. In some embodiments, the weather component 1625 can provide, to the displacing mobile response unit 1640, information that pertains to the weather event. In some embodiments, the weather component 1625 can cause the displacing mobile response unit 1640 to display at least a portion of the information that pertains to the weather event

In step 2325, the weather component 1625 can determine that the route is impacted by the weather event. For example, the weather component 1625 can determine that the weather event has caused a road closure. The road closure can impact the route (e.g., the route includes the road that is closed). The weather component 1625 can provide, to the route generator 1615, an indication that route is impacted by the weather event. The route generator 1615 can update the route. In some embodiments, the route is updated to avoid the weather event. The route generator 1615 can provide, to the route transfer component 1610, the update route. The route transfer component 1610 can provide, to the displacing mobile response unit 1640, the updated route. In some embodiments, the route transfer component 1610 can provide, to the displacing mobile response unit 1640, an alert that the route has been updated. The alert can cause the displacing mobile response unit 1640 to display a prompt to accept the updated route (e.g., the view updated route prompt 2207). The route transfer component 1610 can receive, from the displacing mobile response unit 1640, an indication that the operator of the displacing mobile response unit 1640 has selected the prompt (e.g., the operator selected the accept icon 2210).

In some embodiments, the weather component 1625 can determine that the weather event will impact the receiving facility 1650. For example, the weather component 1625 can determine that a hurricane will cause flooding near the receiving facility 1650. The weather component 1625 can provide, to the machine learning component 1630, an indication that the weather event will impact the receiving facility 1650. The machine learning component 1630, responsive to receiving the indication from the weather component 1625, can identify at least one new facility (e.g., at least one of the receiving facilities 1006) that can, responsive to the weather event impacting the receiving facility 1650, receive the resident and the caretaker associated with the resident. The machine learning component 1630, responsive to identifying the new facility, can provide, to the route generator 1615, the location of the new facility. The route generator 1615, responsive to receiving the location of the new facility, can generate a route that can be taken to reach the new facility. In some embodiments, the route generator 1615 updates the route that was previously provided to the displacing mobile response unit 1640. In some embodiments, the route generator 1615 can provide a new route to the displacing mobile response unit 1640. In some embodiments, the route generator 1615 can update, responsive to the weather event impacting the receiving facility 1650, the route to avoid the receiving facility 1650.

In some embodiments, the machine learning component 1630 can provide, to the displacing mobile response unit 1640, an indication that the weather event will impact the receiving facility 1650. The indication can cause the displacing mobile response unit 1640 to display at least one facility that can receive the resident and the caretaker associated with resident.

In some embodiments, the route transfer component 1610 can provide, to the receiving facility 1650, an indication that the weather event will impact the receiving facility 1650. The indication can include a notice that the resident and the caretaker associated with the resident are no longer in route to the receiving facility 1650.

### Communication System

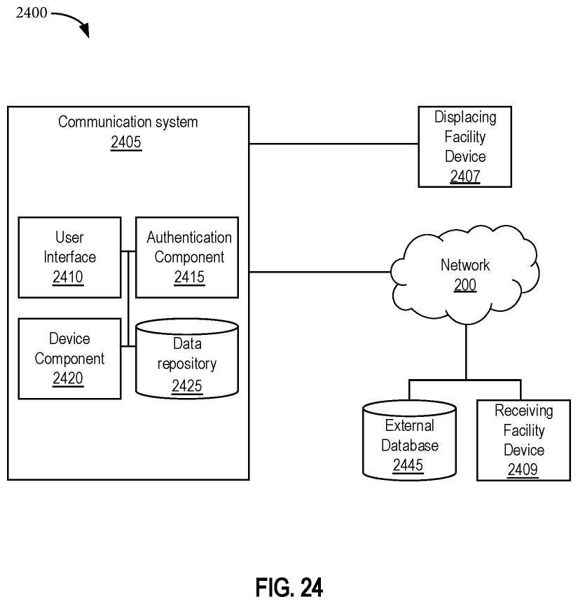

Referring now to FIG. 24, a system 2400 for facilitating communication between devices is shown, according to an exemplary embodiment. The system 2400 can include at least one communication system 2405, at least one displacing facility device 2407, at least one receiving facility device 2409, at least one external database 2445, and the network 200. The communication system 2405 can include at least one user interface 2410, at least one authentication component 2415, at least one device component 2420 and at least one data repository 2425. The communication system 2405 can perform similar functionality to that of the mobile response unit 100. In some embodiments, the communication system 2405 and/or a component of the communication system 2405 can include at least one similar component to that of the mobile response unit 100. In some embodiments, the communication system 2405 can be located at a medical facility (e.g., a hospital, a temporary medical facility, a LTC facility, etc.). In some embodiments, the communication system 2405 can be remote to the medical facility (e.g., not located at the medical facility). In some embodiments, the communication system 2405 can be included within a vehicle (e.g., an ambulance, a medical transport vehicle, a helicopter, a car, etc.). In some embodiments, the displacing facility device 2407 can perform similar functionality to that of the mobile response unit 100 and/or the displacing facility device 2407 can include components similar to the components of the mobile response unit 100. In some embodiments, the displacing facility device 2407 can perform similar functionality to that of the communication system 2405 and/or the displacing facility device 2407 can include components similar to the components of the communication system 2405. In some embodiments, the receiving facility device 2409 can perform similar functionality to that of the mobile response unit 100 and/or the receiving facility device 2409 can include components similar to the components of the mobile response unit 100. In some embodiments, the receiving facility device 2409 can perform similar functionality to that of the communication system 2405 and/or the receiving facility device 2409 can include components similar to the components of the communication system 2405.

The user interface 2410 can facilitate user interaction with the communication system 2405. For example, the user interface 2410 can display, create, provide, or otherwise present information to a user. In some embodiments, the user can be a caretaker that is associated with a resident of a facility. The user interface 2410 can receive information from the user and the user interface 2410 can provide the information to the communication system 2405. In some embodiments, the user interface 2410 can display a prompt requesting a user to enter personal identification information. For example, the prompt can include a request to provide at least one of a personal identification number (PIN), a username and a password associated with the username, a passcode that was provided to a user device that is associated with the user (e.g., a one-time passcode that was provided to the user device 202), an user identification number, or biometric information (e.g., a retinal scan, fingerprint, etc.). In some embodiments, the user interface 2410 can include a scanner. The scanner can detect, determine, or otherwise identify information that is associated with the user. For example, the scanner could detect, from a user identification badge, the user identification number. In some embodiments, the user interface 2410, responsive to receiving the personal identification information, can provide, to the authentication component 2415, the personal identification information.

In some embodiments, the user has previously performed a setup process. The setup process can include creating at least one of an account, a portfolio, or a file with the communication system 2405. In some embodiments, the setup process includes providing the personal identification information to the communication system 2405. In some embodiments, the communication system 2405 can provide the personal identification information to the data repository 2425 and/or the external database 2445. The data repository 2425 and/or the external database 2445 can store, maintain, or otherwise keep the personal identification information.

The authentication component 2415, responsive to receiving the personal identification information, can determine that the user is authorized to interact with the communication system 2405. In some embodiments, the authentication component 2415 can communicate with at least one of the data repository 2425 and/or the external database 2445 in order to receive the personal identification information. In some embodiments, the authentication component 2415 can compare the personal identification information received from the user interface 2410 with the personal identification information received from the data repository 2425 and/or the personal identification information received from the external database 2445. The authentication component 2415, responsive to determining that the personal identification information matches, can authenticate the user. The authentication component 2415 can provide an indication, to at least one component of the communication system 2405, that the user has been authenticated. In some embodiments, the user interface 2410 can display that the user has been authenticated.

In some embodiments, the user can use the displacing facility device 2407 to interface, interact or otherwise communicate with the communication system 2405. For example, the user can provide, via the displacing facility device 2407, the personal identification information to the communication system 2405. In some embodiments, the displacing facility device 2407 can include at least one a smartphone, a tablet, a laptop computer, a desktop computer, and/or any other mobile and/or stationary computing device. In some embodiments, the displacing facility device 2407 can be the user device 202. In some embodiments, all of the information provided, via the user interface 2410, to the communication system 2405 can be provided by the displacing facility device 2407, the receiving facility device 2409, the user device 202 and/or any other external system or device.

The device component 2420 can interface, interact, or otherwise communicate with at least one of the displacing facility device 2407, the receiving facility device 2409 and/or the external database 2445. For example, the device component 2420 can receive, from the displacing facility device 2407, a user's personal identification information. In some embodiments, the device component 2420 can provide a Wi-Fi signal to the displacing facility device 2407 and/or the receiving facility device 2409. The device component 2420 can provide, to the displacing facility device 2407, access to at least one record associated with at least one resident of a displacing facility. For example, the device component 2420 can provide, to the displacing facility device 2407, the record that is associated with the resident. In some embodiments, the record associated with the resident is stored in at least one of the data repository 2425 and/or the external database 2445. In some embodiments, the record includes information similar to that shown in FIGS. 11A and 11B.

The device component 2420 can receive, from the displacing facility device 2407, a request to provide information associated with at least one receiving facility. In some embodiments, the information can include a number of available beds at the receiving facility. In some embodiments, the information associated with the receiving facility is stored in at least one of the data repository 2425 and/or the external database 2445. In some embodiments, the device component 2420 can receive, from the receiving facility device 2409, the information associated with the receiving facility. The device component 2420 can provide, to the displacing facility device 2407, the information associated with the receiving facility.

The device component 2420 can receive, from the displacing facility device 2407, an indication of a transfer of the resident, associated with the displacing facility, to the receiving facility. In some embodiments, the indication of the transfer of the resident can include the operator of the displacing facility device 2407 selecting and/or identifying a receiving facility. In some embodiments, the operator of the displacing facility device 2407 can select the receiving facility using the information associated with the receiving facility. For example, the operator can select the receiving facility that has a certain amount of available beds, a particular type of room available (e.g., an intensive care unit (ICU), a particular type of equipment (an X-Ray machine), a particular type of caretaker (e.g., a surgeon, a nurse, etc.), and/or a particular location. In some embodiments, the information associated with the receiving facility can include the information described herein. The device component 2420, responsive to receiving the indication of the transfer of the resident, can provide, to the receiving facility device 2409, a prompt to accept the transfer of the resident. In some embodiments, the device component 2420 can provide, to the receiving facility device 2409, a signal that causes the receiving facility device 2409, via a user interface, to display the prompt to accept the transfer of the resident.

The device component 2420 can receive, from the receiving facility device 2409, an indication to accept the transfer of the resident. In some embodiments, the device component 2420 can update the information associated with the receiving facility to indicate that a bed has been designated for the resident that will be transferred to the receiving facility. The device component 2420 can, responsive to receiving the indication to accept the transfer of the resident, provide, to the receiving facility device 2409, the record associated with resident. In some embodiments, the device component can provide an indication to at least one of the data repository 2425 and/or the external database 2445 that the receiving facility has accepted the transfer of the resident. The device component 2420, the data repository 2425 and/or the external database 2445 can update the record associated with the resident to include an indication that the resident will be transferred to the receiving facility that accepted the transfer of the resident.

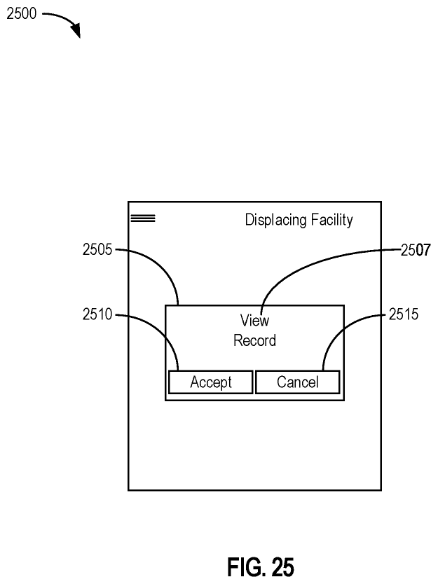

Referring now to FIG. 25, a user interface 2500 is shown, according to an exemplary embodiment. The user interface 2500 can be generated, provided, presented, shown, or otherwise displayed by at least one of the communication system 2405, the displacing facility device 2407, the receiving facility device 2409 and/or the user device 202. In some embodiments, the communication system 2405 can provide signals to at least one the displacing facility device 2407, the receiving facility device 2409 or the user device 202. The signals can cause a device to display the user interface 2500. For example, the device component 2420 can provide signals that cause the displacing facility device 2407 to display the user interface 2500. In some embodiments, the device component 2420 provides the signals to the displacing facility device 2407 responsive to the authentication component 2415 authenticating the displacing facility device 2407. In some embodiments, a mobile application can be stored on the displacing facility device 2407 and the mobile application can cause the displacing facility device 2407 to display the user interface 2500. Similarly, a mobile application can be stored on the receiving facility device 2409 and the mobile application can cause the receiving facility device 2409 to display the user interface 2500.

The user interface 2500 can include at least one window 2505. The window 2505 can be or include at least one of a dialog box, a dialog window, a display window, a display area, or a prompt including an indication. The window 2505 can include at least one view record prompt 2507, at one accept icon 2510 and at least one cancel icon 2515. An operator of the device, that is displaying the user interface 2500, can select the accept icon 2510 in order to view the record that has been provided by the communication system 2405. In some embodiments, the operator of the displacing facility device 2407 can be provided at least one record that is associated with at least one resident of a displacing facility, responsive to the operator selecting the accept icon 2510. In some embodiments, the record can be displayed within the user interface 2500, as an overlay on top of user interface 2500 or a new user interface can be generated. In some embodiments, a user interface similar to that shown in FIG. 11A and/or FIG. 11B can be generated and/or information similar to that shown in FIG. 11A and/or FIG. 11B can be shown in the user interface 2500. The operator of the displacing facility device 2407 can select the cancel icon 2515 to remove the window 2505 and/or to cancel a request to be provided with at least one record.

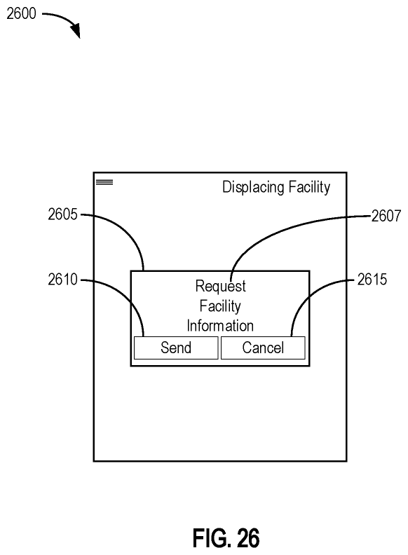

Referring now to FIG. 26, a user interface 2600 is shown, according to an exemplary embodiment. The user interface 2600 can be generated, provided, presented, shown, or otherwise displayed by at least one of the communication system 2405, the displacing facility device 2407, the receiving facility device 2409 and/or the user device 202. The user interface 2600 can include at least one window 2605. The window 2605 can be or include at least one of a dialog box, a dialog window, a display window, a display area, or a prompt including an indication. The window 2605 can include at least one request facility information prompt 2607, at least one send icon 2610 and at least one cancel icon 2615.

The user interface 2600 can be generated responsive to the device component 2420 providing, to the displacing facility device 2407, the record associated with the resident. The window 2605 can include information that indicates that an operator can send a request to the communication system 2405 to provide information associated with at least one receiving facility. The operator can select the send icon 2610 to complete the request to provide the information associated with at least one receiving facility. The operator can select the cancel icon 2615 to cancel the request, to provide the information associated with at least one receiving facility, and/or to remove the window 2605.

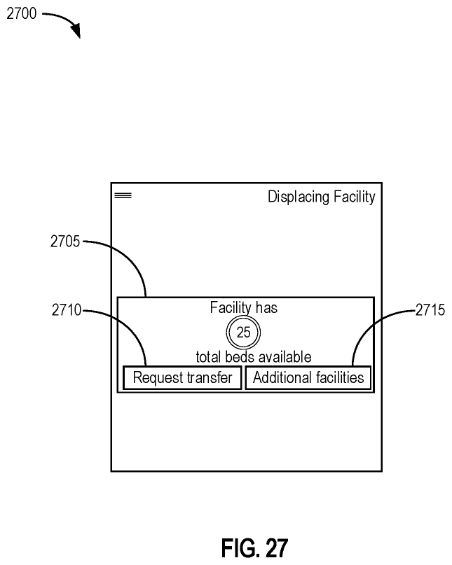

Referring now to FIG. 27, an example user interface 2700 is shown, according to an exemplary embodiment. In some embodiments, the user interface 2700 is generated responsive to at least one of the operator selecting the send icon 2610 and/or the communication system 2405 providing the information associated with at least one receiving facility. In some embodiments, the user interface 2700 can be overlay that is superimposed on top of at least one of the user interface 2500 and or the user interface 2600. The user interface 2700 can include at least one window 2705, at least one request transfer icon 2710 and at least one additional facilities icon 2715.

The window 2705 can be or include at least one of a dialog box, a dialog window, a display window, a display area, or a prompt including an indication. The window 2705 can include the information that is associated with at least one receiving facility. For example, the window 2705 can include a number of total beds available. The operator of the displacing facility device 2407 can select the request transfer icon 2710 to provide an indication, to the communication system 2405, of a transfer of the resident to the receiving facility. The operator can select the additional facilities icon 2715 to be provided information associated with a subsequent receiving facility. In some embodiments, responsive to the operator selecting the additional facilities icon 2715, the device component 2420 can interact with at least one of the data repository 2425 and/or the external database 2445 to receive the information associated with the subsequent receiving facility. In some embodiments, the user interface 2700 can be updated, changed and/or altered to include the information associated with the subsequent receiving facility. In some embodiments, a new user interface can be generated.

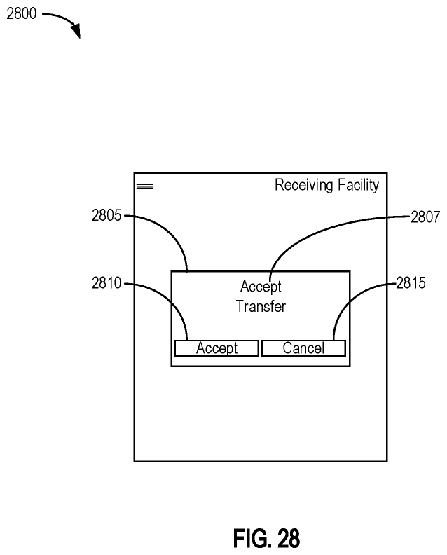

Referring now to FIG. 28, an example user interface 2800 is shown, according to an exemplary embodiment. In some embodiments, the user interface 2800 can be generated, provided, presented, shown, or otherwise displayed by at least one the communication system 2405, the displacing facility device 2407, the receiving facility device 2409 and/or the user device 202. In some embodiments, the user interface 2800 is generated responsive to the operator of the displacing facility device 2407 selecting the request transfer icon 2710. In some embodiments, the user interface 2800 is generated responsive to the communication system 2405 providing, to the receiving facility device 2409, the indication of the transfer of the resident to the receiving facility.

The user interface 2800 can include at least one window 2805. The window 2805 can be or include at least one of a dialog box, a dialog window, a display window, a display area, or a prompt including an indication. The window 2805 can include at least one accept transfer prompt 2807, at least one accept icon 2810 and at least one cancel icon 2815. The window 2805 can include information that pertains to the transfer of a resident. For example, the window 2805 can display an indication to accept a transfer of a resident. In some embodiments, the operator of the receiving facility device 2409 can select the accept icon 2810 to accept the transfer of the resident to the receiving facility. In some embodiments, at least one of the communication system 2405, the displacing facility device 2407 and/or the user device 202 can be provide with an indication that the transfer of the resident to the receiving facility has been accepted. The operator can select the cancel icon 2815 to cancel the request to transfer the resident to the receiving facility and/or to remove the window 2805. In some embodiments, at least one of the communication system 2405, the displacing facility device 2407 and/or the user device 202 can receive an indication that the request to transfer the resident to the receiving facility has been cancelled.

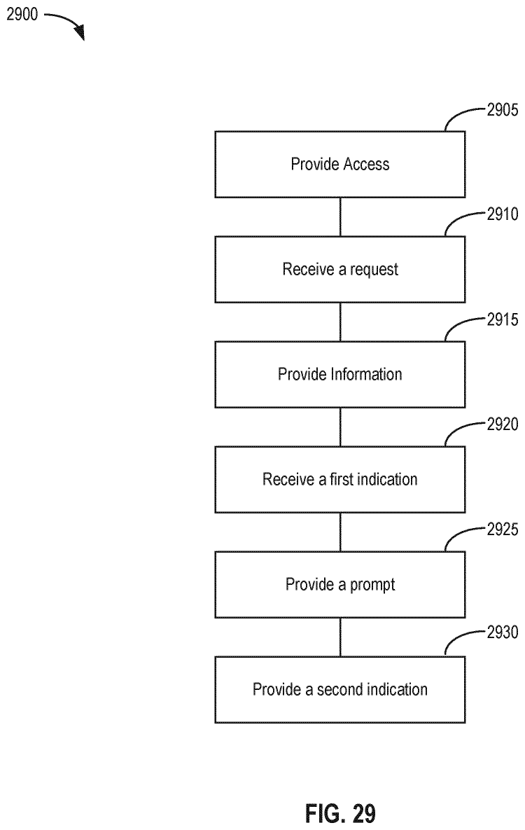

Referring now to FIG. 29, a method 2900 for facilitating communication between devices is shown, according to an exemplary embodiment. In some embodiments, at least one step of the method 2900 can be performed by the communication system 2405, the displacing facility device 2407, the receiving facility device 2409 and/or a component of the system 2400. For example, the device component 2420 can perform at least one step of the method 2900. In some embodiments, a facility that currently houses at least one resident can be impacted by at least one weather event. The weather event can result in at least one resident being displaced from their current facility. The method 2900 described herein can facilitate a seamless transfer of both the resident and at least one record associated with the resident.

In step 2905, the device component 2420 can provide access. In some embodiments, the device component 2420 can provide access to at least one record associated with a resident of a displacing facility. In some embodiments, the device component 2420 can provide access, to the record associated with the resident, to at least one of the displacing facility device 2407, the receiving facility device 2409 and/or the user device 202. In some embodiments, the device component 2420 can provide a continuous or a semi-continuous signal that causes the displacing facility device 2407 to display, via a user interface, the record associated with the resident. The device component 2420, by providing the signal to cause the displacing facility device 2407 to display the record associated with resident, enables the operator to view the record associated with the resident without the need for the displacing facility device 2407 to store, keep, hold, or otherwise maintain a local copy of the record. Furthermore, a device that otherwise would be unable to maintain a local copy of the record can still be provided with the record associated with the resident.

In step 2910, the device component 2420 can receive a request. In some embodiments, the device component 2420 can receive a request to provide information associated with at least one receiving facility. In some embodiments, the device component 2420 can receive the request from at least one of the displacing facility device 2407, the receiving facility device 2409 and/or the user device 202. The information associated with the receiving facility can include a number of beds at the receiving facility. The information associated with the receiving facility can also include at least one of a list of equipment located at the receiving facility, a list of particular types of rooms available at the receiving facility, a list of types of caretaker (e.g., a surgeon, a nurse, etc.) located at the receiving facility, and/or a location of the receiving facility.

In some embodiments, the device component 2420 can receive the request to provide information associated with the receiving facility responsive to an operator, of at least one of the displacing facility device 2407, the receiving facility device 2409 and/or the user device 202, selecting the send icon 1810. The device component 2420, responsive to receiving the request to provide information associated with the receiving facility, can communicate with at least one of the data repository 2425 and/or the external database 2445. The device component 2420, responsive to communicating with the data repository 2425 and/or the external database 2445, can receive the information associated with the receiving facility.

In step 2915, the device component 2420 can provide the information. In some embodiments, the device component can provide, to at least one of the displacing facility device 2407, the receiving facility device 2409 and/or the user device 202, the information associated with the receiving facility device. In some embodiments, the device component 2420 can provide a continuous or semi-continuous signal that causes the displacing facility device 2407 to display the information associated with the receiving facility. For example, the device component 2420 can cause the displacing facility device 2407 to display the information displayed in at least one of the user interface 1000 and/or the user interface 2700. Similar to the signal provided in step 2905, the signal provided to the displacing facility device 2407 allows the displacing facility device 2407 to display the information associated with the receiving facility without the displacing facility storing a local copy of the information associated with the receiving. Additionally, the signal provided by the device component 2420 allows for the information associated with the receiving facility to be immediately updated responsive to the communication system 2405 receiving an indication that the information associated with the receiving facility has changed (e.g., a room located at the receiving facility has been reserved).

In step 2920, the device component 2420 can receive a first indication. In some embodiments, the device component 2420 can receive the first indication from at least one of the displacing facility device 2407, the receiving facility device 2409 and/or the user device 202. In some embodiments, the first indication can be an indication of a transfer of the resident to the receiving facility. In some embodiments, the device component 2420 can receive the first indication responsive to an operator of the displacing facility device 2407 selecting the request transfer icon 2710. In some embodiments, the first indication can include identifying information associated with the receiving facility (e.g., a facility name and a facility location, etc.). The device component 2420 can identify the receiving facility based in part by at least one of the identifying information associated with the receiving facility and/or the information displayed in the user interface 1900.

In step 2925, the device component 2420 can provide a prompt. The prompt can be a prompt to accept first indication of the transfer of the resident from step 2920. In some embodiments, the device component 2420 can provide the prompt to at least one of the receiving facility device 2409 and/or the user device 202. In some embodiments, the device component 2420, responsive to providing the prompt, can cause the receiving facility device 2409 to display the user interface 2000.

in step 2930, the device component 2420 can receive a second indication. The device component 2420 can receive a second indication to accept the transfer of the resident. In some embodiments, the device component 2420 can receive the second indication responsive to an operator of the receiving facility device 2409 selecting the accept icon 2010. In some embodiments, the device component 2420 can provide, to the receiving facility device 2409, the record associated with the resident that was previously provided to the displacing facility device 2407. In some embodiments, the device component 2420 can provide a third indication, to the displacing facility device 2407, that the receiving facility has accepted the transfer of the resident. In some embodiments, the device component 2420 can update the record associated with the resident to include a fourth indication that resident will be transferred to the receiving facility. The device component 2420 can provide the updated record associated with the resident to at least one of the data repository 2425 and/or the external database 2445.

In some embodiments, the displacing facility device 2407, the receiving facility device 2409 and/or the user device can include, store, hold or otherwise maintain at least one mobile application. The mobile application can be or include at least one of a software application, a computer program and/or any other possible application. The mobile application can enable a device (e.g., the displacing facility device 2407) to interface with the mobile response unit 100 and/or perform similar functionality to that of the mobile response unit 100. For example, the displacing facility device 2407, responsive to storing the mobile application, can provide, to the receiving facility device 2409 a prompt to accept a transfer of a resident, from the displacing facility, to the receiving facility. The mobile application can be or include a web-based application. The web-based application can allow a device (e.g., the user device 202) to perform similar functionality to that of the mobile response unit 100 without an operator of the user device 202 having to first store the mobile application of the user device 202.

### Wearable Device

During times of emergency the residents of a facility may need to be evacuated. During the evacuation of the residents, the records (e.g., medical records) associated with the residents must accompany the residents. The resident can be provided a physical copy of their records. For example, the records can be printed and then provided to the resident. However, residents may not be able to keep track of their records, or the residents are incapable of reliably ensuring that their records are not lost. The wearable record device (e.g., wearable device) described herein provides a technical solution where the records associated with a resident can be stored by the wearable device. Advantageously, the wearable device enables a displacing facility (e.g., the facility impacted by an emergency) to seamlessly and efficiently provide the records associated with the resident to the receiving facility. For example, when the resident arrives at a receiving facility the wearable device can provide, to the receiving facility, the records associated with the resident.

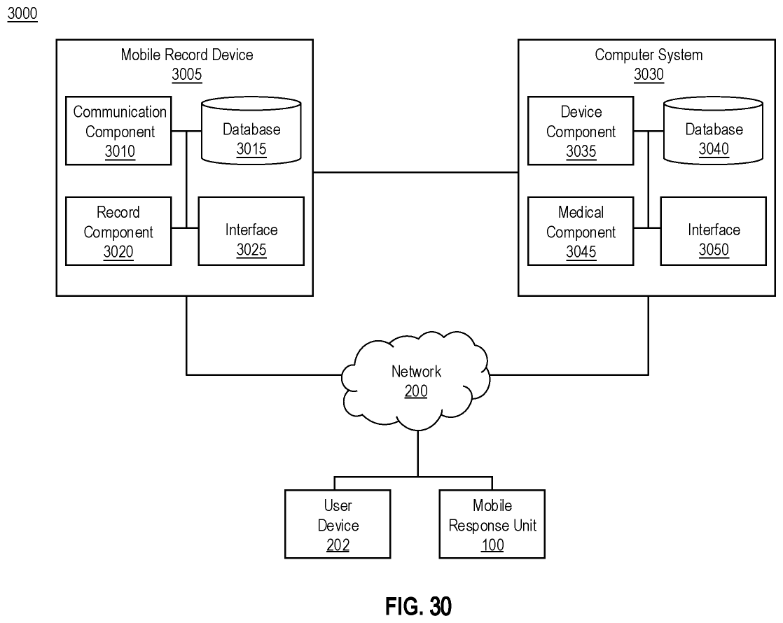

Referring now to FIG. 30, a system 300 is shown, according to an exemplary embodiment. The system 3000 can include at least one mobile record device 3005 (e.g., the wearable device described herein), at least one computer system 3030, the network 200, the mobile response unit 100, and at least one user device 202. The mobile record device 3005 can be or include at least one of a watch, a band, a strap, a bracelet, a necklace, a Carabiner, an airtag, a belt, a ring, a lanyard, a piece of clothing, a mobile phone, a cell phone, a tablet, a laptop, a pair of glasses, wristband or any other possible device that can be wearable. For example, the mobile record device can be a strap. The strap can include at least one hole and at least one pin. The hole can receive, accept, obtain, acquire, or otherwise encounter the pin. The pin can be mounted, attached, secured, or coupled with the hole. The strap, responsive to the pin coupling with the hole, can be mounted, attached, secured, or coupled with an object. The object can be at least one of a person (including the body parts of the person), a backpack, a wheelchair, a vehicle, a crutch, a cast, a neck brace, a purse, a keychain, or any other possible household object. For example, the strap can be coupled with a wrist of a person.

The mobile record device 3005 can include at least one communication component 3010, at least one database 3015, at least one record component 3020 and at least one interface 3025. In some embodiments, at least one component of the mobile record device 3005 includes similar components to that of the mobile response unit 100. In some embodiments, the mobile record device 3005 can perform similar functionality to that of the mobile response unit 100. In some embodiments, at least one component of the mobile record device 3005 includes similar components to that of the computer system 3030. In some embodiments, the mobile record device 3005 can perform similar functionality to that of the computer system 3030. In some embodiments, the mobile response unit 100 can perform similar functionality to at least one of the mobile record device 3005 and/or the computer system 3030.

The computer system 3030 can include at least one device component 3035, at least one database 3040, at least on medical component 3045 and at least one interface 3050. In some embodiments, at least one component of the computer system 3030 includes similar components to that of the mobile response unit 100. In some embodiments, the computer system 3030 can perform similar functionality to that of the mobile response unit 100.

The communication component 3010 can interface, interact or otherwise communicate with at least one of the computer system 3030, the device component 3035, the user device 202 and/or any additional system or device (e.g., the mobile response unit 100). In some embodiments, the communication component 3010 can receive, from the device component 3035, an indication to associate the mobile record device 3005 with at least one resident of a facility. The indication can include at least one of the name of the resident, the name of the facility, the location of the facility and/or any other possible information that can identify at least one of the resident and/or the facility. In some embodiments, the communication component 3010 can receive the indication responsive to an operator of the computer system 3030 selected a prompt displayed by the interface 3050. The interface 3050 can generate, display, provide or otherwise present at least one prompt. The prompt can include a section where the operator can select, enter, or otherwise choose a resident. For example, the operator can select the resident from a drop down menu. The communication component 3010 can interface, interact, or otherwise communicate, responsive to receiving the indication to associate the mobile record device 3005 with the resident of the facility, with the record component 3020.

The record component 3020, responsive to receiving the indication to associate the mobile record device 3005 with the resident, can associate the mobile record device 3005 with the resident. In some embodiments, the record component 3020 can associate the mobile record device 3005 with the resident by linking, paring, joining, relating, or otherwise connecting the mobile record device 3005 with the resident. The record component 3020 can provide, to the database 3015, confirmation that the mobile record device 3005 has been associated with the resident. The database 3015 can store, hold, or otherwise maintain information that indicates that the mobile record device 3005 has been associated with the resident.

In some embodiments, the record component 3020 can provide, to at least one of the interface 3025 and/or the communication component 3010, the confirmation that the mobile record device 3005 has been associated with resident. In some embodiments, the communication component 3010, responsive to receiving the confirmation from the record component 3020, can provide, to the device component 3035, confirmation that the mobile record device 3005 has been associated with the resident. In some embodiments, the device component 3035 can provide, to the interface 3050, a signal that causes the interface 3050 to display a notice. The notice can include a statement that the mobile record device 3005 has been associated with the resident. The interface 3050 can generate, display, provide or otherwise present at least one prompt.

The record component 3020, responsive to the mobile record device 3005 being associated with the resident, can provide, to the communication component 3010, a request to receive at least one record associated with the resident. In some embodiments, the request can include the name of the resident. The record can include at least one of a medical record, emergency contact information and/or resident activity logs (e.g., when the resident last ate, when the resident was last checked on, etc.). The communication component 3010 can provide, to the device component 3035, the request to receive the record associated with the resident. In some embodiments, the communication component 3010 can include the request to receive the record associated with the resident with the confirmation that the mobile record device 3005 has been associated with the resident.

The device component 3035, responsive to receiving the request to receive the record associated with the resident, can communicate with the database 3040. The database 3040 can provide, to the device component 3035, the record associated with the resident. The database 3040 can store, hold, keep, or otherwise maintain at least one record associated with at least one resident. The device component 3035, responsive to receiving the record associated with the resident, can provide, to the communication component 3010, the record associated with the resident. The communication component 3010 can provide, to the database 3015, the record associated with the resident. The database 3015 can, responsive to receiving the record associated with the resident, store the record associated with the resident.

The medical component 3045 can include at least one of a heart rate monitor, a pulse oximeter, a blood pressure monitor, an electrocardiogram (ECG) monitor, a glucose monitor and/or a temperature monitor (e.g., a thermometer). The medical component 3045 can perform at least one health check on the resident associated with the mobile record device 3005. In some embodiments, the health check can include taking at least one measurement and/or performing at least one test (e.g., a heart rate measurement, a blood pressure measurement, an ECG test, a glucose measurement, a temperature measurement, etc.). For example, an operator of the computer system 3030 can take a blood pressure measurement of a resident. The medical component 3045 can provide the health check, measurements associated with the health check and/or tests associated with the health check to at least one of the device component 3035, the database 3040 and/or the interface 3050.

The communication component 3010 can receive, from the device component 3035, an indication. In some embodiments, the indication can include information associated with the resident. The information can include the health check that was performed by the medical component 3045. In some embodiments, the indication can include that a health check of the resident was performed. The communication component 3010, responsive to receiving the indication, can provide the information associated with the resident to at least one of the record component 3020, the database 3015 and/or the interface 3025.

The record component 3020 can receive, from the communication component 3010, the information associated with the resident. The record component 3020, responsive to receiving the information associated with the resident, can communicate with the database 3015. The database 3015 can provide, to the record component 3020, the record associated with the resident. In some embodiments, the record component 3020 can compare the information associated with the resident with the record associated with the resident. In some embodiments, the record component 3020 can determine that the information associated with the resident and the record associated with the resident are different. For example, when the mobile record device 3005 previously received the record associated with the resident the record can include a heat rate measurement. The information associated with the resident can include the health check performed by the medical component 3045. The record component 3020 can determine that the heart rate measurement included in the record associated with the resident is different than a heart rate measurement included in the health check. The record component 3020, responsive to determining that the record associated with the resident and the information associated with the resident are different, can update the record associated with the resident. For example, the record component 3020 can update the record associated with the resident to include the heart rate measurement that was include in the health check. The record component 3020, responsive to updating the record associated with the resident, can provide the updated record associated with the resident to the database 3015.

In some embodiments, the record component 3020 can automatically, responsive to receiving the information associated with the resident, update the record associated with the resident. For example, as described herein the information associated with the resident, which was provided by the device component 3035, can include that a health check was performed by the medical component 3045. The record component 3020, responsive to determining that the information associated with the resident includes the health check can automatically update the record associated with the resident to include the health check.

The communication component 3010 can interface, interact, or otherwise communicate with the user device 202. In some embodiments, the user device is associated with at least one of the receiving facility (e.g., the facility that is receiving the resident) and/or a caretaker associated with the resident (e.g., the caretaker assigned to assist the resident). The communication component 3010 can provide, to the user device 202, the record associated with the resident. In some embodiments, the communication component 3010 can provide, to the user device 202, the record associated with the resident responsive to the communication component 3010 receiving, form the user device 202, a request to receive the record associated with the resident.

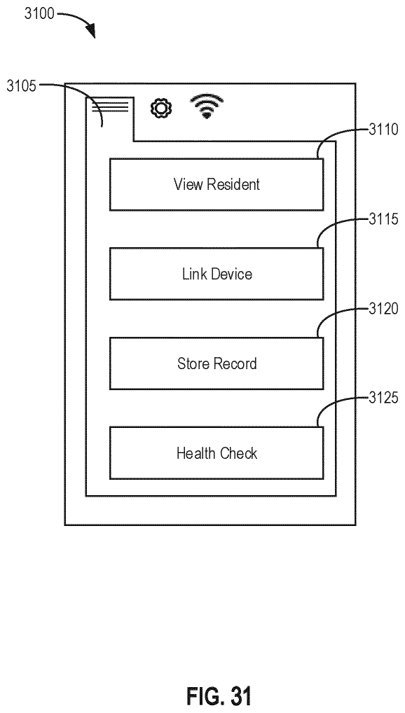

Referring now to FIG. 31, an example user interface 3100 is shown, according to an exemplary embodiment. The user interface 3100 can be generated, provided, presented, shown, or otherwise displayed by the mobile record device 3005. For example, the interface 3025 can display the user interface 3100. In some embodiments, the computer system 3030 and/or the user device 202 can display the user interface 3100. The user interface 3100 can include at least one menu 3105, at least one view resident icon 3110, at least one link device icon 3115, at least one store record icon 3120 and at least one health check icon 3125. In some embodiments, the user interface 3100 can display information that was provided by the computer system 3030. For example, the user interface 3100 can display the record associated with the resident. In some embodiments, the user interface 3100 can display information similar to that shown in FIGS. 11A and 11B.

An operator of the mobile record device 3005 can select the view resident icon 3110 to view the resident that the computer system 3030 included in the indication to associate the mobile record device 3005 with the resident. For example, the user interface 3100 can display the name of the resident included in the indication. The operator, responsive to viewing the resident name, can confirm that the resident name displayed in the user interface 3100 matches the resident that will be associated with the mobile record device 3005.

An operator of the mobile record device 3005 can select the link device icon 3115 to associate the mobile record device 3005 with the resident that was displayed responsive to the operator selecting the view resident icon 3110. In some embodiments, the user interface 3100 can display a prompt indicating that the operator of the mobile record device 3005 can accept the indication to associate the mobile record device 3005 with the resident.

An operator of the mobile record device 3005 can select the store record icon 3120 to store the record, provided by the computer system 3030, associated with the resident. In some embodiments, the user interface 3100 can include the store record icon responsive to the mobile record device 3005 receiving the record associated with the resident.

An operator of the mobile record device 3005 can select the health check icon 3125 to view and/or perform a health check. For example, the medical component 3045 can perform a health check responsive to the operator of the computer system 3030 selecting the health check icon 3125. In some embodiments, the user interface 3100 can display at least one of a drop down menu, a list or an otherwise grouping of measurements and or tests that can be performed or included in the health check.

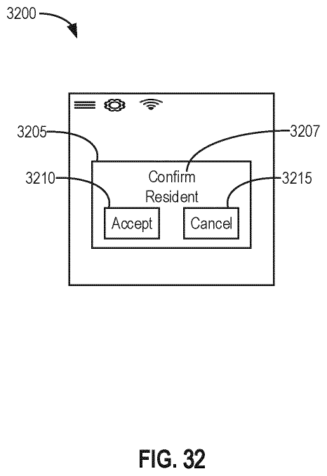

Referring now to FIG. 32, an example user interface 3200 is shown, according to an exemplary embodiment. The user interface 3200 can be displayed by the mobile record device 3005 and/or the computer system 3030. In some embodiments, the user interface 3200 is generated responsive to an operator of the mobile record device 3005 selecting the view resident icon 3110. In some embodiments, the user interface 3200 is displayed as an overlay on top of the user interface 3100. In some embodiments, the information displayed in the user interface 3200 is combined with the information displayed in the user interface 3100. In some embodiments, a new user interface is generated to display the information associated with the user interface 3200.

The user interface 3200 can include a window 3205. The window 3205 can be or include at least one of a dialog box, a dialog window, a display window, a display area, or a prompt including an indication. The window 3205 can include a confirm resident prompt 3207, an accept icon 3210 and a cancel icon 3215. An operator of the mobile record device 3005 can select the accept icon 3210 to confirm the resident that was included in the indication to associate the mobile record device 3005 with the resident. The operator of the mobile record device 3005 can select the cancel icon 3215 to cancel the indication to associate the mobile record device 3005 with the resident or to remove the window 3205.

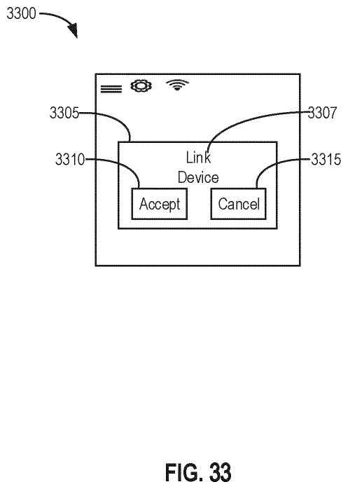

Referring now to FIG. 33, an example user interface 3300 is shown, according to an exemplary embodiment. In some embodiments, the user interface 3300 can be generated responsive to the operator of the mobile record device 3005 selecting the accept icon 3210. In some embodiments, the user interface 3300 can be generated responsive to an operator selecting the link device icon 3115. The user interface 3300 can include a window 3305. The window 3305 can be or include at least one of a dialog box, a dialog window, a display window, a display area, or a prompt including an indication. The window 3305 can include a link device prompt 3307, an accept icon 3310 and a cancel icon 3315. In some embodiments, the operator of the mobile record device 3005 can select the accept icon 3310 to link the mobile record device 3005 with the resident that was confirmed by the operator selecting the accept icon 3210. The operator of the mobile record device 3005 can select the cancel icon 3315 to cancel the mobile record device 3005 being associated with the resident or to remove the window 3305.

In some embodiments, the interface 3025, responsive to the operator selecting the accept icon 3210, can provide confirmation, to the record component 3020, that the mobile record device 3005 has been associated with the resident. The record component 3020 can provide, to database 3015, the confirmation that mobile record device 3005 has been associated with the resident. The database 3015 can maintain the confirmation.

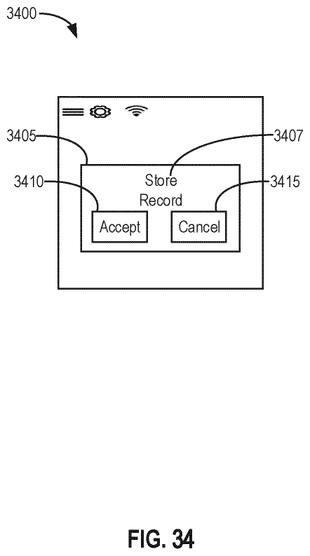

Referring now to FIG. 34, an example user interface 3400 is shown, according to an exemplary embodiment. The user interface 3400 can be generated responsive to the computer system 3030 providing, to the mobile record device 3005, the record associated with the resident. The user interface 3400 can include a window 3405. The window 3405 can be or include at least one of a dialog box, a dialog window, a display window, a display area, or a prompt including an indication. The window 3405 can include a store record prompt 3407, an accept icon 3410 and a cancel icon 3415. In some embodiments, the user interface 3400 can be generated responsive to an operator selecting the store record icon 3120 and/or the operator selecting the accept icon 3410.

In some embodiments, the interface 3025, responsive to the operator of the mobile record device 3005 selecting the accept icon 3410, can provide, to the database 3015, an indication to the store the record associated with the resident. The database 3015, responsive to receiving the indication from the interface 3025, can store the record associated with the resident. The interface 3025, responsive to the operator of the mobile record device 3005 selecting the cancel icon 2015, can provide, to the database 3015, an indication to not store the record associated with the resident.

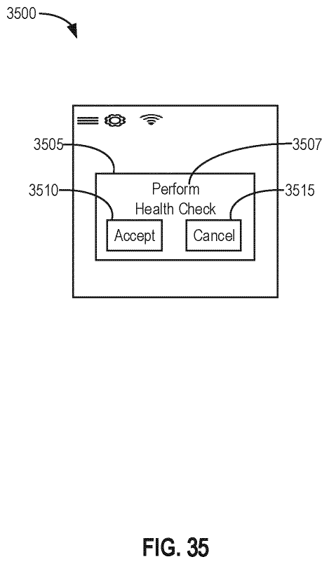

Referring now to FIG. 35, an example user interface 3500 is shown, according to an exemplary embodiment. The user interface 3500 can be generated responsive to an operator selecting the health check icon 3125. The user interface 3500 can include a window 3505. The window 3505 can be or include at least one of a dialog box, a dialog window, a display window, a display area, or a prompt including an indication. The window 3505 can include a perform health check prompt 3507, an accept icon 3510 and a cancel icon 3515. In some embodiments, the user interface 3500 can also include a list of measurements and or tests that can be performed and/or include in the health check.

The interface 3050, responsive to an operator of the computer system 3030 selecting the accept icon 3510, can provide a signal to the medical component 3045. The medical component 3045 can perform, responsive to receiving the signal from the interface 3050, at least one health check. In some embodiments, the information associated with the health check can be displayed in the user interface 3500. For example, the user interface 3500 can display a heart rate measurement. The operator of the computer system 3030 can select the cancel icon 3515 to cancel a request to perform a health check or to remove the window 3505.

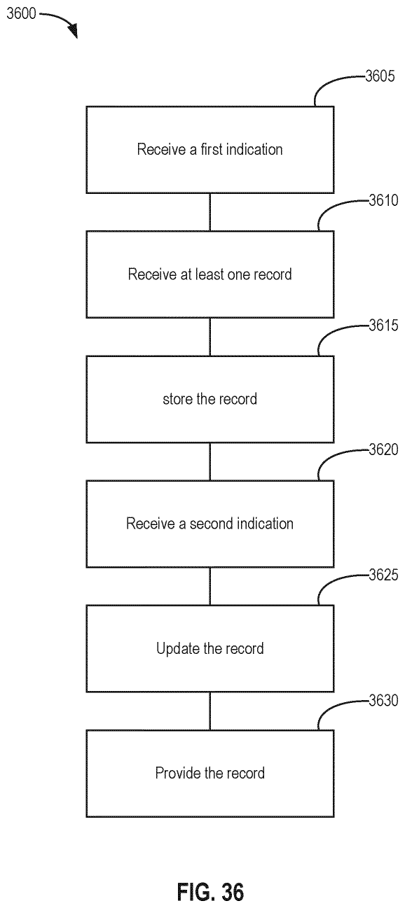

Referring now to FIG. 36, a method 3600 for associating a mobile record device with a resident is shown, according to an exemplary embodiment. In some embodiments, at least on step of the method 3600 can be performed by the mobile record device 3005, the mobile response unit 100 and/or a component of the mobile record device 3005.

In step 3605, the communication component 3010, can receive a first indication. In some embodiments, the communication component 3010 can receive the first indication from the computer system 3030 and/or the device component 3035. The first indication can be an indication to associate the mobile record device 3005 with a resident of a facility. In some embodiments, the communication component 3010 can provide, to the record component 3020, the first indication to associate the mobile record device 3005 with the resident.

In some embodiments, the record component 3020, responsive to receiving the first indication to associate the mobile record device 3005 with the resident, can associate the mobile record device 3005 with the resident. In some embodiments, the record component 3020 can provide, to the database 3015 and/or the communication component 3010, confirmation that the mobile record device 3005 was associated with the resident.

In step 3610, the communication component 3010 can receive at least one record. In some embodiments, the record can be associated with the resident that the mobile record device 3005 is associated with. In some embodiments, the communication component 3010 can receive the record associated with the resident responsive to the communication component 3010 providing, to the computer system 3030, confirmation that the mobile record device 3005 is associated with the resident.

In some embodiments, the computer system 3030 receives the record associated with the resident responsive to an operator of the mobile record device 3005 selecting the link device icon 3115 and/or the operator selecting the accept icon 3310. In some embodiments, the communication component 3010 can provide, to the interface 3025, the record associated with the resident. In some embodiments, the interface 3025, responsive to receiving the record associated with the resident can display the record associated with the resident and/or information associated with the record. The communication component 3010 can provide, to the database 3015, the record associated with the resident.

In step 3615, the database 3015 can store the record associated with the resident. In some embodiments, the database 3015 can automatically store, responsive to receiving the record associated with the resident, the record associated with the resident. In some embodiments, the database 3015 stores the record associated with the resident responsive to an operator selecting the store record icon 3120 and/or the accept icon 3410.

In step 3620, the communication component 3010 can receive a second indication. In some embodiments, the second indication can include information associated with the resident. For example, the information associated with the resident can include a health check. The communication component 3010, responsive to receiving the second indication, can provide, to the record component 3020, the information associated with the resident.

The record component 3020, responsive to receiving the information associated with the resident, can receive, from the database 3015, the record associated with the resident. The record component 3020 can compare the record associated with the resident with the information associated with resident. In some embodiments, the record component 3020 can determine a difference between the record associated with the resident and the information associated with the resident. For example, the record associated with the resident can include a previous test (e.g., an ECG test). In some embodiments, the information associated with the resident can include a health check. The health check can include an ECG test.

In step 3625, the record component 3020 can update the record associated with the resident. In some embodiments, the record component 3020, responsive to determining a difference between the ECG test included in the record associated with the resident and the ECG test included in the health check, can update the record associated with the resident to include the ECG that was included in the health check. The record component 3020, responsive to updating the record associated with the resident to include the health check, can provide, to the database 3015, the updated record associated with the resident. The database 3015, responsive to receiving the updated record associated with the resident, can store the updated record.

In step 3630, the communication component 3010, can provide the record associated with the resident. In some embodiments, the communication component 3010 can provide the record associated with the resident to a user device (e.g., user device 202) that is associated with at least one of a receiving facility (e.g., a facility that will receive the resident) and/or a caretaker associated with the resident. In some embodiments, the communication component 3010 can cause the user device 202 to display, responsive to the user device 202 receiving the record associated with the resident, the record associated with the resident.

### Communication System

During times of emergency the residents of a facility may need to be evacuated. The evacuation of the residents from the facility includes relocating a resident from a displacing facility (e.g., the facility impacted by an emergency) to a receiving facility (e.g., the facility that will receive the resident). Furthermore, the evacuation of the residents provides challenges to both the displacing facility and the receiving facility. In particular, the displacing facilities and/or the staff of the displacing facilities are tasked with identifying the residents located at the displacing facility, associating the residents with at least one record (e.g., medical record) and then relocating the residents and the records associated with the residents to the receiving facility. These tasks can take several hours to complete and during times of emergency, the displacing facilities do not have the time to complete these tasks.

The systems and methods described herein enable a displacing facility to provide a request to a communication system and the communication system can identify at least one resident located at the displacing facility, identify at least one record associated with the resident and provide the request to at least one receiving facility that was identified by the communication system. Advantageously, the communication system enables a displacing facility (e.g., the facility impacted by an emergency) to seamlessly and efficiently request the transfer of residents.

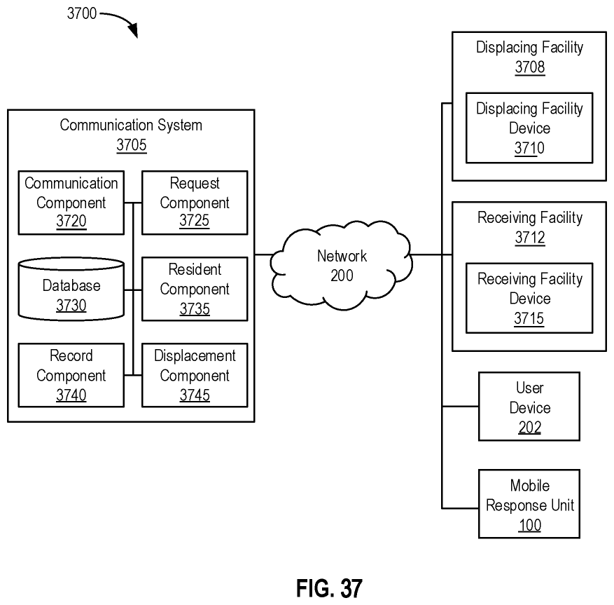

Referring now to FIG. 37, a system 3700, for facilitating communication between one or more facilities, is shown according to an exemplary embodiment. The system 3700 can include at least one communication system 3705, at least one displacing facility 3708, at least one receiving facility 3712, the user device 202, the network 200, and the mobile response unit 100. The communication system 3705 can include at least one communication component 3720, at least one request component 3725, at least one database 3730, at least one resident component 3735, at least one record component 3740 and at least one displacement component 3745. In some embodiments, at least one component of the communication system 3705 includes similar components to that of the mobile response unit 100. In some embodiments, the communication system 3705 can have similar functionality to that of the mobile response unit 100. In some embodiments, the mobile response unit 100 can perform similar functionality to the communication system 3705. In some embodiments, the mobile response unit 100 can include the communication system 3705. In some embodiments, the communication system 3705 and/or the mobile response unit 100 can provide, to one or more devices (e.g., the user device 202), internet access using at least one of TV whitespace and/or Starlink. In some embodiments, the communication system 3705 and/or the mobile response unit 100 can include a headphone jack (e.g., a component that can receive, accept, couple, or obtain a headphone or similar device).

The displacing facility 3708 can include at least one displacing facility device 3710. The displacing facility device 3710 can be at least one of a smartphone, a tablet, a laptop computer, a desktop computer, a mobile response unit 100 and/or any other mobile and/or stationary computing device. The receiving facility 3712 can include at least one receiving facility device 3715. The receiving facility device 3715 can be at least one of a smartphone, a tablet, a laptop computer, a desktop computer, a mobile response unit 100 and/or any other mobile and/or stationary computing device.

The communication component 3720 can interface, interact or otherwise communicate with at least one of the displacing facility 3708, the displacing facility device 3710, the receiving facility 3712, the receiving facility device 3715 and/or the user device 202. The communication component 3720 can receive, from the displacing facility 3708, a request to transfer a resident associated with the displacing facility 3708. The request can include at least one record associated with the resident. The communication component 3720, responsive to receiving the request, can provide, to the request component 3725, the request. The request component 3725, responsive to receiving the request, can communicate with the database 3730.

The database 3730 can store, hold or otherwise maintain at least one of a list of resident associated with at least one facility, at least one record associated with the resident, a list including at least one facility, information associated with the facilities, weather data and/or at least one predetermined displacement metric. The information associated with the facilities can include at least one of an address of each facility, contact information associated with each facility (e.g., a phone number, an email address, etc.), a total number of beds available at each facility and/or a list of services available at each facility (e.g., the facility can perform X-rays, the facility can administer medication, etc.). In some embodiments, the list of residents can include at least one resident associated with at least one of the displacing facility 3708 and/or the receiving facility 3712. In some embodiments, the list of facilities can include the displacing facility 3708 and the receiving facility 3712. In some embodiments, the information associated with the facilities can be updated continuously or semi-continuously. For example, the communication component 3720 can communicate with the facilities included in the list and the facilities can provide, to the communication component 3720, updated information associated with the facilities. The communication component 3720 can provide, to the database 3730, the updated information associated with the facilities. The database 3730 can store the updated information associated with facilities.

The request component 3725 can receive, from the database 3730, the list of the facilities, the information associated with the facilities, and the record associated with the resident. The request component 3725, using at least one of the request, the list of the facilities, the information associated with the facilities and/or the record associated with the resident, can identify at least one facility that can be a receiving facility (e.g., the receiving facility 3712). The request component 3725, responsive to identifying the receiving facility 3712, can provide, to the communication component 3720, the request to transfer the resident associated with the displacing facility 3708 and the information associated with the identified receiving facility 3712.

The communication component 3720 can, responsive to receiving the request and the information associated with the identified receiving facility 3712, provide, to the receiving facility 3712, the request to transfer the resident associated with the displacing facility 3708. In some embodiments, the communication component 3720 providing the request, to the receiving facility 3712, can cause, the receiving facility device 3715, to display, via a user interface associated with the receiving facility device 3715, a prompt to accept the request to transfer the resident associated with the displacing facility 3708.

The communication component 3720, can receive, from the receiving facility 3712, an indication to accept the request to transfer the resident associated with the displacing facility 3708. In some embodiments, the communication component 3720 can receive the indication to accept the request responsive to an operator of the receiving facility device 3715 selecting an icon to accept a prompt to accept the request. The communication component 3720 can provide, to the resident component 3735, the indication to accept the request.

The resident component 3735, responsive to receiving the indication to accept the request to transfer the resident associated with the displacing facility 3708, can associate the resident with the receiving facility 3712. The resident component 3735 can associate the resident with the receiving facility 3712 by performing at least one of linking, grouping, connecting, relating, coupling, or otherwise uniting the resident with the receiving facility 3712. The resident component 3735, responsive to associating the resident with the receiving facility 3712, can provide, to the record component 3740, a notice that the resident has been associated with the receiving facility 3712. The resident component 3735, responsive to receiving the notice, can communicate with the database 3730. The database 3730, responsive to communicating with the record component 3740, can provide, to the record component 3740, the record associated with the resident.

The record component 3740, responsive to receiving the record associated with the resident, can update the record associated with the resident. The record component 3740 can update the record associated with the resident by including, in the record associated with the resident, that the resident has been associated with the receiving facility 3712. The record component 3740 can also update the record associated with the resident to include an indication that the resident is no longer associated with the displacing facility 3708. The record component 3740 can provide the updated record associated with the resident to the database 3730. The database 3730, responsive to receiving the updated record associated with the resident, can store the updated record associated with the resident. The record component 3740 can provide, to the communication component 3720, the record associated with the resident.

The communication component 3720, responsive to receiving the record associated with the resident, can provide, to the receiving facility 3712, the record associated with the resident. The displacement component 3745, responsive to the communication component 3720 providing, to the receiving facility 3712, the record associated with the resident, can communicate with the database 3730. The database 3730, responsive to communicating with the displacement component 3745, can provide, to the displacement component 3745, the record associated with the resident.

The displacement component 3745 can, using at least one of the record associated with the resident and/or the predetermined displacement metric, identify at least one person associated with at least one of the receiving facility 3712 and/or the resident. The displacement component 3745, responsive to identifying the person associated with at least one of the receiving facility 3712 and/or the resident, can identify a user device (e.g., the user device 202) associated with the identified person. The displacement component 3745 can provide, to the communication component 3720, information associated with the identified person.

The communication component 3720, responsive to receiving the information associated with the identified person, can provide, to the user device 202 associated with the identified person, an alert that the resident will be transferred to the receiving facility 3712. The alert can include information associated with at least one of the receiving facility 3712 and/or the resident. The alert, responsive to the communication component 3720 providing, to the user device 202 associated with the identified person, can cause, via a user interface associated with the user device 202, the user device 202 to display the information associated with at least one of the receiving facility 3712 and/or the resident.

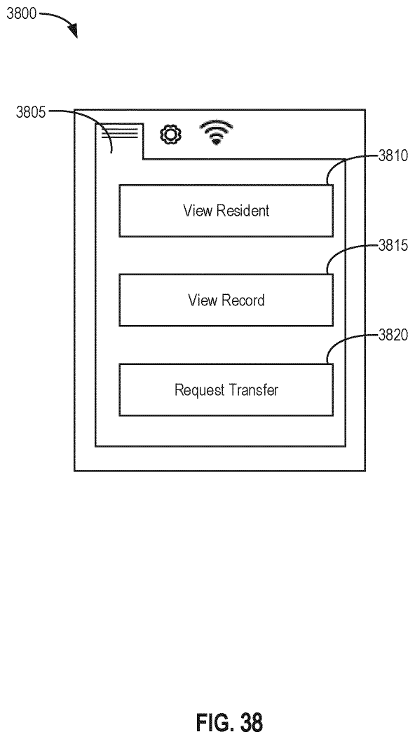

Referring now to FIG. 38, an example user interface 3800 is shown, according to an exemplary embodiment. In some embodiments, at least one of the displacing facility device 3710, the receiving facility device 3715 and/or the user device 202 can generate, provide, present, or otherwise display, via a user interface, the user interface 3800. The user interface 3800 can include a menu 3805. The menu 3805 can be at least one of a drop down menu, a dashboard menu, a menu that is an overlay on top of the user interface 3800 and/or any other possible menu. The menu 3805 can include a view resident icon 3810, a view record icon 3815 and a request transfer icon 3820. In some embodiments the user interface 3800 is accessible via an online portal (e.g., a web application, etc.). In some embodiments, the user interface 3800 is accessible via a mobile application (e.g., an application that has been stored on the device accessing the user interface 3800). In some embodiments, the communication system 3705 can provide, to at least one of the displacing facility device 3710, the receiving facility device 3715 and/or the user device 202, a signal that causes at least one of the devices to display the user interface 3800.

An operator of the device displaying the user interface 3800 can be provided, responsive to the operator selecting the view resident icon 3810, a list of at least one resident associated with at least one of the displacing facility 3708 and/or the receiving facility 3712. For example, an operator of the displacing facility device 3710 can select the view resident icon 3810. The user interface 3800 can, responsive to the operator selecting the view resident icon 3810, generate, provide, present, or otherwise display the list of residents associated with at least one of the displacing facility 3708 and/or the receiving facility 3712. In some embodiments, the communication system 3705 can receive an indication that the operator has selected the view resident icon 3810. The communication system 3705, responsive to receiving the indication, can provide the list of the residents associated with at least one of the displacing facility 3708 and/or the receiving facility 3712. In some embodiments, the list of the residents associated with at least one the displacing facility 3708 and/or the receiving facility 3712 is stored locally (e.g., is stored on the device displaying the user interface 3800).

An operator of the device displaying the user interface 3800 can be provided, responsive to the operator selecting the view record icon 3815, at least one record associated with at least one resident. For example, an operator of the displacing facility device 3710 can select the view record icon 3815. The user interface 3800 can, responsive to the operator selecting the view record icon 3815, generate, provide, present, or otherwise display the record associated with the resident. In some embodiments, the operator can be prompted to provide the name of the resident prior to the user interface 3800 displaying the record associated with the resident.

In some embodiments, the communication system 3705 can receive an indication that the operator has selected the view record icon 3815 The communication system 3705, responsive to receiving the indication, can provide the record associated with the resident. In some embodiments, the record associated with the resident is stored locally (e.g., is stored on the device displaying the user interface 3800). In some embodiments, the user interface 3800 can display information similar to that shown in FIGS. 11A and/or 11B.

An operator of the device displaying the user interface 3800 can select the request transfer icon 3820 to provide, to the communication system 3705, a request to transfer the resident associated with the displacing facility 3708. In some embodiments, the operator can be prompted to provide the name of the resident prior to the request to transfer the resident associated with the displacing facility 3708 being provided to the communication system 3705.

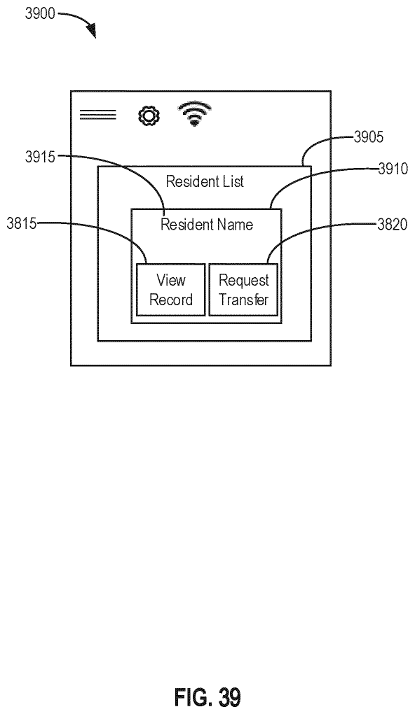

Referring now to FIG. 39, a user interface 3900 is shown, according to an exemplary embodiment. In some embodiments, the user interface 3900 is generated responsive to an operator of the device displaying the user interface 3800 selecting the view resident icon 3810. In some embodiments, the user interface 3900 is displayed as overlay on top of the user interface 3800. In some embodiments, the user interface 3900 can include the information included in the user interface 3800. In some embodiments, the user interface 3900 replaces the user interface 3800. The user interface 3900 includes at least one resident list window 3905. The resident list window 3905 can be or include at least one of a dialog box, a dialog window, a display window, a display area, or a prompt including an indication. The resident list window 3905 includes at least one resident profile 3910. In some embodiments, the resident profile 3910 can be displayed as at least one of an icon, a window, an account, a prompt and/or a text box. The resident profile 3910 can include a resident name 3915. The resident profile 3910 can include the view record icon 3815 and the request transfer icon 3820.

The operator of the device displaying the user interface 3900 can select the view record icon 3815 to view the record associated with the resident profile 3910. In some embodiments, the user interface 3900, responsive to the operator selecting the view record icon 3815, can display information similar to that shown in FIGS. 11A and/or 11B. The operator of the device displaying the user interface 3900 can select the request transfer icon 3820 to provide, to the communication system 3705, a request to transfer the resident associated with the resident profile 3910.

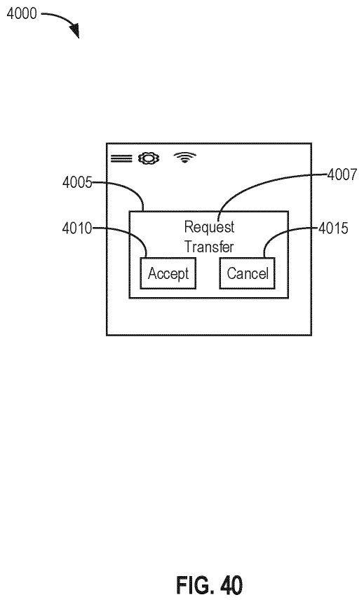

Referring to FIG. 40, a user interface 4000 is shown, according to an exemplary embodiment. In some embodiments, the user interface 4000 is generated responsive to an operator of the device displaying at least one of the user interface 3800 and/or the user interface 3900 selecting the request transfer icon 3820. In some embodiments, the user interface 4000 is displayed as an overlay on top of at least one of the user interface 3800 and/or the user interface 3900. In some embodiments, at least one of the displacing facility device 3710, the receiving facility device 3715 and/or the user device 202 can generate, provide, present, or otherwise display, via a user interface, the user interface 4000.

The user interface 4000 can include a window 4005. The window 4005 can be or include at least one of a dialog box, a dialog window, a display window, a display area, or a prompt including an indication. The window 4005 includes a request transfer prompt 4007, an accept icon 4010 and a cancel icon 4015. In some embodiments, the operator of the device displaying the user interface 4000 can select the accept icon 4010 to complete the request to transfer the resident associated with the displacing facility 3708. For example, the communication system 3705 receives the request to transfer the resident associated with the displacing facility 3708 responsive to the operator selecting the accept icon 4010. The operator can select the cancel icon 4015 to cancel the request to transfer the resident associated with the displacing facility 3708.

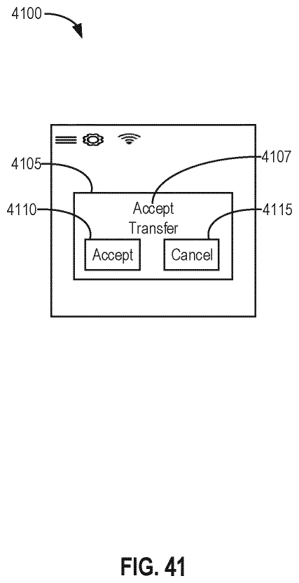

Referring now to FIG. 41, a user interface 4100 is shown, according to an exemplary embodiment. In some embodiments, the user interface 4100 is generated responsive to an operator of the device displaying at least one of the user interface 3800 and/or the user interface 3900 selecting the request transfer icon 3820 and the communication system 3705 providing, to the receiving facility 3712, the request. In some embodiments, the user interface 4100 is displayed as an overlay on top of at least one of the user interface 3800, the user interface 3900 and/or the user interface 4000. In some embodiments, at least one of the displacing facility device 3710, the receiving facility device 3715, the mobile response unit 100 and/or the user device 202 can generate, provide, present, or otherwise display, via a user interface, the user interface 4100.

The user interface 4100 can include a window 4105. The window 4105 can be or include at least one of a dialog box, a dialog window, a display window, a display area, or a prompt including an indication. The window 4105 can include an accept transfer prompt 4107, an accept icon 4110 and a cancel icon 4115. An operator of the device displaying the user interface 4100 can select the accept icon 4110 to provide, to the communication system 3705, an indication to accept the request to transfer the resident associated with the displacing facility 3708. The operator can select the cancel icon 4115 to provide, to the communication system 3705, an indication that the receiving facility 3712 has cancelled the request to transfer the resident associated with the displacing facility 3708.

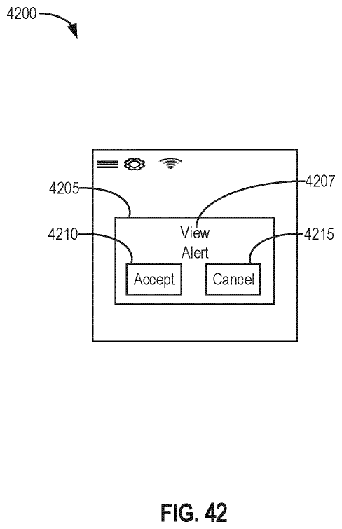

Referring now to FIG. 42, a user interface 4200 is shown, according to an exemplary embodiment. In some embodiments, the user interface 4200 is generated responsive to an operator of the device displaying the user interface 4100 selecting the accept icon 4110. In some embodiments, the user interface 4200 is displayed as an overlay on top of at least one of the user interface 3800, the user interface 3900, the user interface 4000 and/or the user interface 4100. In some embodiments, at least one of the displacing facility device 3710, the receiving facility device 3715 and/or the user device 202 can generate, provide, present, or otherwise display, via a user interface, the user interface 4200. The user interface 4200 can include a window 4205. The window 4205 can be or include at least one of a dialog box, a dialog window, a display window, a display area, or a prompt including an indication. The window 4205 can include a view alert prompt 4207, an accept icon 4210 and a cancel icon 4215. An operator of the select the accept icon 4210 to view information associated with at least one of the receiving facility 3712 and/or the resident.

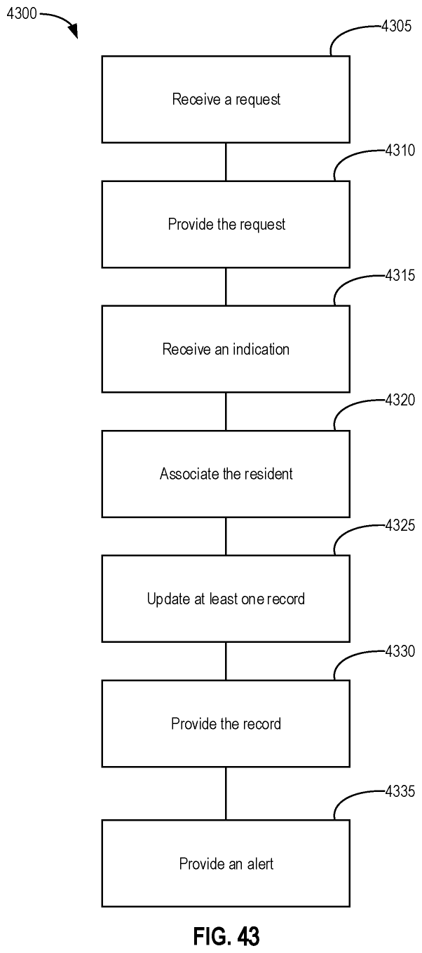

Referring now to FIG. 43, a process 4300 for facilitating communication between one or more facilities is shown, according to an exemplary embodiment. In some embodiments, at least one step of the process 4300 can be performed by the communication system 3705, the mobile response unit 100 and/or a component of the system 3700. In some embodiments, at least one component of the communication system 3705, for example, the communication component 3720 can perform at least one step of the process 4300.

In step 4305, the communication component 3720, can receive a request. The communication component 3720 can receive the request from the displacing facility 3708 and/or the displacing facility device 3710. In some embodiments, the communication component 3720 can receive the request, responsive to an operator of the displacing facility device 3710 selecting the request transfer icon 3820 and or the accept icon 4010. The request can be a request to transfer a resident associated with the displacing facility 3708. The communication component 3720, responsive to receiving the request can provide, to the request component 3725, the request.

The request component 3725, responsive to receiving the request, can receive, from the database 3730, at least one of the list of the facilities, the information associated with the facilities, and the record associated with the resident. The request component 3725, using at least one of the request, the list of the facilities, the information associated with the facilities and/or the record associated with the resident, can identify at least one facility that can be a receiving facility (e.g., the receiving facility 3712). The request component 3725, responsive to identifying the receiving facility 3712, can provide, to the communication component 3720, the request to transfer the resident associated with the displacing facility 3708 and the information associated with the identified receiving facility 3712.

In step 4310, the communication component 3720, can provide the request. The communication component 3720 can provide the request to at least one of the receiving facility 3712 (e.g., the receiving facility identified in step 4305) and/or the receiving facility device 3715 associated with the receiving facility 3712 identified in step 4305. In some embodiments, the request can be the request to transfer the resident associated with the displacing facility 3708. In some embodiments, the communication component 3720 can cause, responsive to providing the request to the receiving facility device 3715, the receiving facility device 3715 to display, via a user interface, a prompt to accept the request. For example, the communication component 3720 can cause the receiving facility device 3715 to display the user interface 4100 and/or the window 4105.

In step 4315, the communication component 3720 can receive an indication. The indication can be an indication to accept the transfer of the resident associated with the displacing facility 3708. The communication component 3720 can receive the indication from at least one of the receiving facility 3712 and/or the receiving facility device 3715. In some embodiments, the communication component 3720 can receive the indication responsive to an operator of the receiving facility device 3715 selecting the accept icon 2010. The communication component 3720 can provide, to the resident component 3735, the indication.

In step 4320, the resident component 3735 can associate the resident. The resident component 3735 can associate the resident with the receiving facility 3712. The resident component 3735 can associate the resident with the receiving facility 3712 by performing at least one of linking, grouping, connecting, relating, coupling, or otherwise uniting the resident with the receiving facility 3712. The resident component 3735, responsive to associating the resident with the receiving facility 3712, can provide, to the record component 3740, a notice that the resident has been associated with the receiving facility 3712. The resident component 3735, responsive to receiving the notice, can communicate with the database 3730. The database 3730, responsive to communicating with the record component 3740, can provide, to the record component 3740, the record associated with the resident.

In step 4325, the record component 3740 can update at least one record. In some embodiments, the record component 3740 can update the record associated with the resident. The record component 3740 can update the record associated with the resident by including, in the record associated with the resident, that the resident has been associated with the receiving facility 3712. The record component 3740 can also update the record associated with the resident to include an indication that the resident is no longer associated with the displacing facility 3708. The record component 3740 can provide the updated record associated with the resident to the database 3730. The database 3730, responsive to receiving the updated record associated with the resident, can store the updated record associated with the resident. The record component 3740 can provide, to the communication component 3720, the record associated with the resident.

In step 4330, the communication component 3720 can provide the record. In some embodiments, the communication component 3720 can provide, to the receiving facility 3712 and/or the receiving facility device 3715, the record. The record can be the record associated with the resident. The displacement component 3745, responsive to the communication component 3720 providing, to the receiving facility 3712, the record associated with the resident, can communicate with the database 3730. The database 3730, responsive to communicating with the displacement component 3745, can provide, to the displacement component 3745, the record associated with the resident.

The displacement component 3745 can, using at least one of the record associated with the resident and/or the predetermined displacement metric, identify at least one person associated with at least one of the receiving facility 3712 and/or the resident. The displacement component 3745, responsive to identifying the person associated with at least one of the receiving facility 3712 and/or the resident, can identify a user device (e.g., the user device 202) associated with the identified person. The displacement component 3745 can provide, to the communication component 3720, information associated with the identified person.

In step 4335, the communication component 3720 can provide an alert. The alert can be an alert that the resident will be transferred to the receiving facility 3712. The alert can include information associated with at least one of the receiving facility 3712 and/or the resident. The alert can be provided to the user device 202 that was identified in step 2230. In some embodiments, the alert can cause the user device 202 to display the information associated with at least one of the receiving facility 3712 and/or the resident. For example, the alert can cause the user device 202 to display the user interface 4200 and/or the window 4205.

### Emergency Preparedness

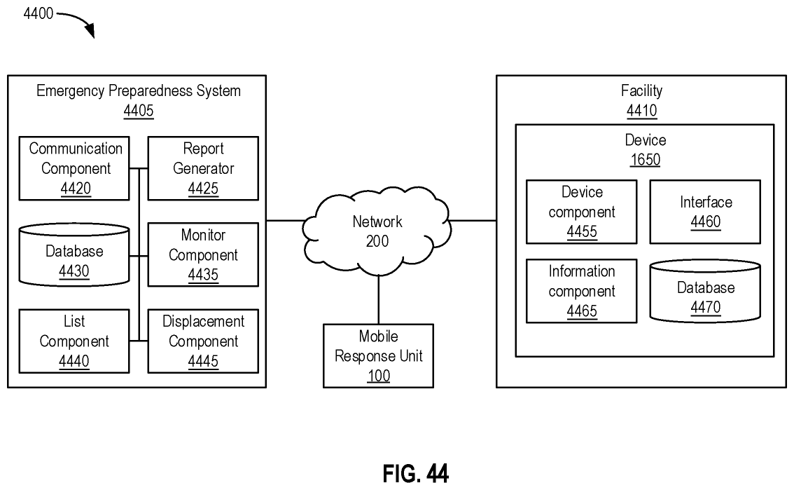

Referring now to FIG. 44, a system 4400 is shown, according to an exemplary embodiment. The system 4400 can include at least one emergency preparedness system (EPS) 4405, at least one facility 4410, the mobile response unit 100, and the network 200. The EPS 4405 can include at least one communication component 4420, at least one report generator 4425, at least one database 4430, at least one monitor component 4435, at least one list component 4440 and at least one displacement component 4445. The facility 4410 can include at least one device 4450. The device 4450 can include at least one device component 4455, at least one interface 4460, at least one information component 4465 and at least one database 4470. The device 4450 can be at least one of a mobile response unit (e.g., the mobile response unit 100) and/or a user device (e.g., the user device 202). In some embodiments, the EPS 4405 can perform similar functionality to that of the mobile response unit 100. In some embodiments, at least one component of the EPS 4405 is similar to at least one component of the mobile response unit 100. In some embodiments, the mobile response unit 100 can perform similar functionality to the EPS 4405. In some embodiments, the mobile response unit 100 can include the EPS 4405.

The information component 4465 can generate, detect, collect, gather, identify, track or otherwise determine information that pertains to at least one of the facility 4410 and/or the device 4450. The interface 4460 can provide, to the information component 4465, data that has been entered by an operator of the device 4450. The information component 4465 can identify, using the data provided by the interface 4460, information that pertains to the facility 4410. The information, that pertains to at least one of the facility 4410 and/or the device 4450, can include at least one of facility configuration (security system, fire alarm system, temperature control, facility resources, energy systems, etc.), equipment setup (e.g., equipment configuration, equipment status, equipment upgrades, software downloads, software updates, etc.), employee training and/or evacuation protocols (e.g., emergency procedures, evacuation plans, etc.). For example, an operator of the device 4450 can enter, using the interface 4460, that the facility 4410 has ten vehicles (e.g., facility resources).

Additionally, the operator of the device 4450 can enter, using the interface 4460, an evacuation plan. Additionally, the information component 4465 can track the equipment status of the device 4450. For example, the device 4450 can include a predetermined equipment setup process. The equipment setup process can include at least one step. The information component 4465 can determine when one of the steps has been completed, executed, or otherwise performed. The information component 4465 can provide, to the device component 4455, the information that pertains to the facility 4410 and/or the device 4450. The device component 4455 can interact, interface, or otherwise communicate with the communication component 4420.

The communication component 4420 can interact, interface, or otherwise communicate with the device 4450 and/or the device component 4455. The communication component 4420 can receive, from the device component 4455, the information that pertains to the facility 4410 and/or the device 4450. The communication component 4420, responsive to receiving the information that pertains to the facility 4410 and/or the device 4450, can provide, to the report generator 4425, the information that pertains to the facility 4410 and/or the device 4450.

The report generator 4425, responsive to receiving the information that pertains to the facility 4410 and/or the device 4450, can communicate with the database 4430. The database 4430 can store, hold, or otherwise maintain at least one of a predetermined standard, information that pertains to at least one additional facility and/or one additional device (e.g., a second facility 4410 and/or a second device 4450). In some embodiments, the database 4430 can provide, to the report generator 4425, the information that pertains to at least one additional facility and/or one additional device.

The report generator 4425, using the information that pertains to the facility 4410 and/or the device 4450, can generate at least one report. In some embodiments the report can be or include at least one emergency preparedness score. The emergency preparedness score can include at least one of a facility score and/or a mobile response unit score. The emergency preparedness score can also include at least one recommendation that can adjust the emergency preparedness score, the facility score and/or the mobile response unit score. The report can include a facility score. The report can include at least one recommendation. The facility score can be a comparison between the facility 4410 and other peer facilities. The facility score can indicate the level of emergency preparedness for the facility 4410 and/or the device 4450. The recommendation can provide an indication of an improvement, an action or a process that could be implemented and/or followed by at least one of the facility 4410 and/or the device 4450. In some embodiments, the implementation of the recommendation can result in the facility score being adjusted, modified, changed, or otherwise updated. The updated facility score can indicate an improved level of emergency preparedness. The report generator 4425, responsive to generator the report, can provide, to the communication component 4420, the report.

The communication component 4420, responsive to receiving the report, can provide, to the device 4450, the report. In some embodiments, the communication component 4420 providing the report to the device 4450 can cause the device 4450 to display, via the interface 4460, the report. The communication component 4420 can receive, from the device 4450, an indication that the facility 4410 accepted the recommendation. In some embodiments, the communication component 4420 can receive the indication responsive to the operator of the device 4450 selecting a prompt to accept the recommendation. The communication component 4420, responsive to receiving the indication that the facility 4410 accepted the recommendation, can provide, to the monitor component 4435, the indication.

The monitor component 4435, responsive to receiving the indication, can monitor the progress of the recommendation. For example, the recommendation can be a recommendation to update the security system of the facility 4410 and the monitor component 4435 can monitor the recommendation by looking for information that indicates that the security system has been updated. The monitor component 4435 can monitor the progress of the recommendation by monitoring information that is provided, by the device 4450, to the communication component 4420. The device 4450 can continuously and/or semi continuously provide, to the communication component 4420, information that pertains to the facility 4410 and/or the device 4450. For example, the monitor component 4435 can monitor the progress of the recommendation to update the security system of the facility 4410 by determining that the information that pertains to the facility 4410 and/or the device 4450 includes information that indicates that the security system of the facility 4410 was updated.

The monitor component 4435 can detect, responsive to receiving, from the communication component 4420, information that pertains to the facility 4410 and/or the device 4450, additional information that pertains to at least one of the facility 4410 and/or the device 4450. In some embodiments, the additional information can include at least one action that was performed at the facility 4410. In some embodiments, the additional information can include at least one action that was performed on the device 4450 and/or at least one action that was performed using the device 4450. The action can include adjusting, modifying and/or otherwise changing the information that pertains to the facility 4410 and/or the device 4450. In some embodiments the action can be at least one of facility improvements (e.g., updating fire alarm system, etc.), additional equipment setup, additional training of employees and/or modification of the evacuation protocol.

The monitor component 4435, responsive to detecting the additional information that pertains to at least one of the facility 4410 and/or the device 4450, can provide, to the report generator 4425, the additional information that pertains to at least one of the facility 4410 and/or the device 4450. The report generator 4425, responsive to receiving the additional information that pertains to at least one of the facility 4410 and/or the device 4450. Can communicate with the database 4430. The database 4430, responsive to communicating with the report generator 4425, can provide, to the report generator 4425, the predetermined standard. In some embodiments, the predetermined standard can include at least one action.

The report generator 4425 can compare the additional information that pertains to at least one of the facility 4410 and/or the device 4450 with the predetermined standard. In some embodiments, the report generator 4425 can determine that the additional information exceeds the predetermined standard. In some embodiments, the additional information can exceed the predetermined standard when the additional information includes at least one action that is not included in the predetermined standard. For example, the predetermined standard can include that action A and action B be performed. The additional information can include that action A and B were performed and the additional information can also include that action C was performed. In some embodiments, the inclusion of action C in the additional information can indicate that the additional information exceeds the predetermined standard. The report generator 4425, responsive to determining that the additional information exceeds the predetermined standard, can update the facility score.

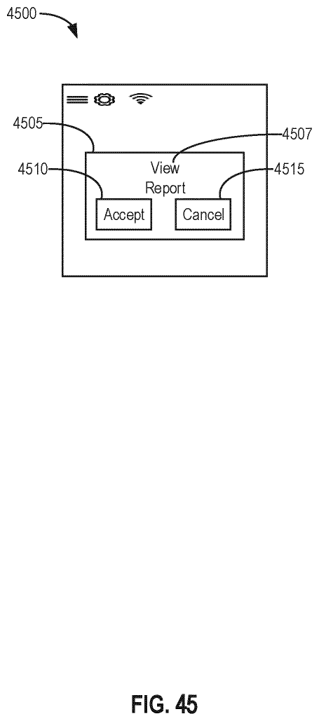

Referring now to FIG. 45, an example user interface 4500 is shown, according to an exemplary embodiment. The user interface 4500 can include a window 4505. The window 4505 can be or include at least one of a dialog box, a dialog window, a display window, a display area, or a prompt including an indication. The window 4505 can include a view report prompt 4507, an accept icon 4510 and a cancel icon 4515. In some embodiments, the user interface 4500 can be generated, provided, presented, or otherwise displayed by the interface 4460. In some embodiments, the user interface 4500 is generated responsive to the communication component 4420 providing, to the device 4450, the report. An operator of the device 4450 can select the accept icon 4510 to view, see or otherwise interact with the report. The operator of the device 4450 can select to cancel icon 4515 to prevent the report from being displayed by the interface 4460.

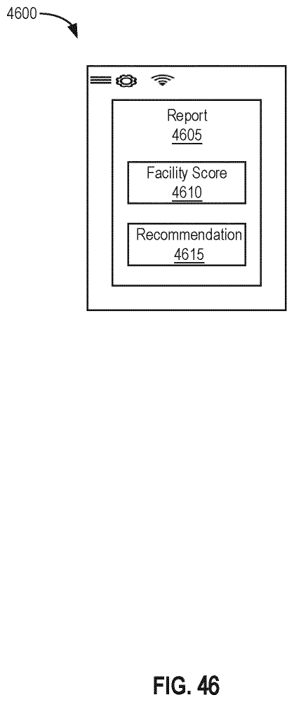

Referring now to FIG. 46, an example user interface 4600 is shown, according to an exemplary embodiment. In some embodiments, the user interface 4600 can include a report 4605. The report 4605 can include at least one emergency preparedness score. FIG. 46 shows the emergency preparedness score as facility score 4610. The emergency preparedness score is also shown to include recommendation 4615. In some embodiments, the user interface 4600 can be generated, provided, presented, or otherwise displayed by the interface 4460. In some embodiments, the user interface 4600 is generated responsive to the communication component 4420 providing, to the device 4450, the report. In some embodiments, the user interface 4600 is generated responsive to an operator of the device 4450 selecting the accept icon 4510. In some embodiments, the user interface 4600 is generated an as overlay on top of the user interface 4500. In some embodiments, the information displayed by the user interface 4500 is combined with the information displayed by the user interface 4600. In some embodiments, the operator of the device 4450 can select the recommendation 4615 to accept the recommendation 4615.

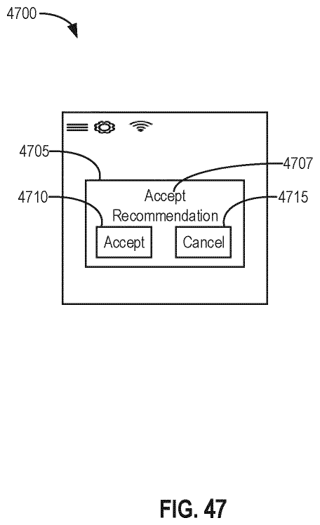

Referring now to FIG. 47, an example user interface 4700 is shown, according to an exemplary embodiment. In some embodiments, the user interface 4700 can include a window 4705. The window 4705 can be or include at least one of a dialog box, a dialog window, a display window, a display area, or a prompt including an indication. The window 4705 can include an accept recommendation prompt 4707, an accept icon 4710 and a cancel icon 4715. In some embodiments, the user interface 4700 can be generated, provided, presented, or otherwise displayed by the interface 4460. In some embodiments, the user interface 4700 is generated responsive to an operator of the device 4450 selecting or otherwise interacting with the recommendation 4615. In some embodiments, the user interface 4700 is generated as an overlay on top of at least one of the user interface 4500 and/or the user interface 4600. In some embodiments, the information displayed by the user interface 4500 and/or the information displayed by the user interface 4600 is combined with the information displayed by the user interface 4700. The operator of the device 4450 can select the accept icon 4710 to accept the recommendation that was included in the report (e.g., the report 4605). In some embodiments, the communication component 4420 can receive, from the device component 4455, an indication that the facility 4410 has accepted the recommendation.

In some embodiments, the mobile response unit 100 and/or the EPS 4405 can generate a report. The report can be or include an emergency preparedness score. The emergency preparedness score can provide an indication to the emergency preparedness of a facility (e.g., facility 4410) and/or a mobile response unit (e.g., the mobile response unit 100). The emergency preparedness score can grade, rank, or otherwise score at least one of actions, initiatives, programs, evacuation measures and/or emergency plans that have been performed by the facility 4410 and/or the mobile response unit 100. For example, the mobile response unit 100 can generate, using a date stamp of the most recent data backup, for an emergency preparedness score. The emergency preparedness score generated by the mobile response unit 100 can indicate that facility 4410 is less prepared for an emergency responsive to determining that the most recent data backup occurred 2 months ago. The emergency preparedness score can also indicate how the emergency preparedness score can be adjusted by performing more frequent data backups.

In some embodiments, the mobile response unit 100 can generate, using an evacuation plan for the facility 4410, an emergency preparedness score for the facility 4410. The emergency preparedness score can indicate that the evacuation plan properly addresses at least one of federal, state and/or local regulations. The emergency preparedness score can also indicate which, if any, of the federal, state, or local regulations that are not addresses based on the current evacuation plan for the facility 4410. The emergency preparedness score can include a recommendation that can address at least one of the federal, state and/or local regulations. For example, the recommendation can be that state regulations suggest that a facility has both a physical copy and a digital copy of the facilities evacuation plan and given that facility 4410 has only a physical copy the emergency preparedness score can be adjusted by also providing a copy to the mobile response unit 100 and/or any other possible computing device.

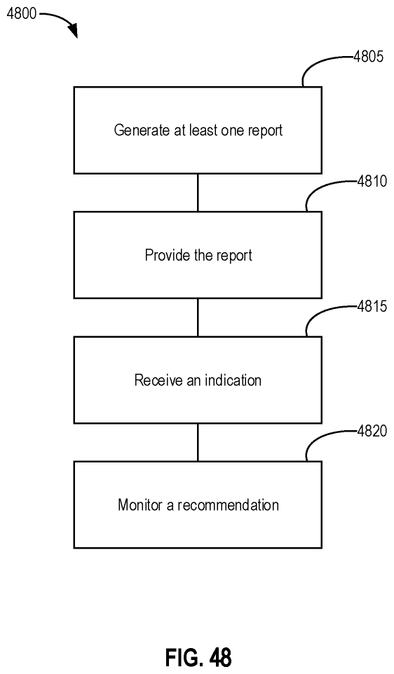

Referring now to FIG. 48, a process 4800 for generating at least one facility score is shown, according to an exemplary embodiment. In some embodiments, at least one step of the process 4800 can performed by the EPS 4405, the mobile response unit 100 and/or a component of the system 1600. In some embodiments, at least one step of the process 4800 can be performed by a component of the EPS 4405 (e.g., the communication component 4420).

In step 4805, the report generator 4425 can generate at least one report. In some embodiments, the report generator 4425 can generate, using information that pertains to a facility (e.g., the facility 4410). In some embodiments, the report can be an emergency preparedness score. The emergency preparedness score can include at least one of a facility score or a mobile response unit score. In some embodiments, the report can include a facility score and the report can include at least one recommendation. The recommendation can be associated with the emergency preparedness of the facility 4410. The report generator 4425 can provide, to the communication component 4420, the report.

In step 4810, the communication component 4420 can provide the report. In some embodiments, the communication component 4420 can provide the report to a device (e.g., the device 4450). In some embodiments, the device 4450, via the interface 4460, can display the report. In some embodiments, the interface 4460 can display a prompt to accept the recommendation. The operator of the device 4450 can select the prompt to provide an indication, to the EPS 4405, that the facility 4410 accepted the recommendation.

In step 4815, the communication component 4420 can receive an indication. In some embodiments, the indication can be an indication that the facility 4410 accepted the recommendation. In some embodiments, the communication component 4420 can receive the indication responsive to an operator the device 4450 selecting the accept icon 190. In some embodiments, the indication can be an indication that the facility 4410 has cancelled the recommendation that was provided in the report.

In step 4820, the monitor component 4435 can monitor a recommendation. In some embodiments, the monitor component 4435 can monitor the recommendation that was accepted by the facility 4410 in step 4815. In some embodiments, the monitor component 4435 can monitor an additional recommendation that was provided to the device 4450. In some embodiments, the monitor component 4435 can monitor the recommendation by monitoring information, provided by the communication component 4420, that pertains to at least one of the facility 4410 and or the device 4450.

The embodiments described herein have been described with reference to drawings. The drawings illustrate certain details of specific embodiments that implement the systems, methods and programs described herein. However, describing the embodiments with drawings should not be construed as imposing on the disclosure any limitations that may be present in the drawings.

It should be understood that no claim element herein is to be construed under the provisions of 35 U.S.C. § 112(f), unless the element is expressly recited using the phrase “means for.”

As used herein, the term “medical record” may include information relating to the care of one or more individuals. Medical records may include physical records (e.g., paper documents, receipts, etc.) and/or electronic records (e.g., electronic medical records (“EMR”), administration records (“eMAR”), electronic treatment authorization request records (“eTAR”), activities of daily living records (“ADL”), etc.). Medical records may include informal records (e.g., physician recommendations, etc.) and/or formal records (e.g., post-operative reports, prescriptions, etc.).

As used herein, the term “circuit” may include hardware structured to execute the functions described herein. In some embodiments, each respective “circuit” may include machine-readable media for configuring the hardware to execute the functions described herein. The circuit may be embodied as one or more circuitry components including, but not limited to, processing circuitry, network interfaces, peripheral devices, input devices, output devices, sensors, etc. In some embodiments, a circuit may take the form of one or more analog circuits, electronic circuits (e.g., integrated circuits (“IC”), discrete circuits, system on a chip (“SOC”) circuits, etc.), telecommunication circuits, hybrid circuits, and any other type of “circuit.” In this regard, the “circuit” may include any type of component for accomplishing or facilitating achievement of the operations described herein. For example, a circuit as described herein may include one or more transistors, logic gates (e.g., NAND, AND, NOR, OR, XOR, NOT, XNOR, etc.), resistors, multiplexers, registers, capacitors, inductors, diodes, wiring, and so on.

The “circuit” may also include one or more processors communicatively coupled to one or more memory or memory devices. In this regard, the one or more processors may execute instructions stored in the memory or may execute instructions otherwise accessible to the one or more processors. In some embodiments, the one or more processors may be embodied in various ways. The one or more processors may be constructed in a manner sufficient to perform at least the operations described herein. In some embodiments, the one or more processors may be shared by multiple circuits (e.g., circuit A and circuit B may comprise or otherwise share the same processor which, in some example embodiments, may execute instructions stored, or otherwise accessed, via different areas of memory). Alternatively or additionally, the one or more processors may be structured to perform or otherwise execute certain operations independent of one or more co-processors. In other example embodiments, two or more processors may be coupled via a bus to enable independent, parallel, pipelined, or multi-threaded instruction execution. Each processor may be implemented as one or more general-purpose processors, application specific integrated circuits (“ASICs”), field programmable gate arrays (“FPGAs”), digital signal processors (“DSPs”), or other suitable electronic data processing components structured to execute instructions provided by memory. The one or more processors may take the form of a single core processor, multi-core processor (e.g., a dual core processor, triple core processor, quad core processor, etc.), microprocessor, etc. In some embodiments, the one or more processors may be external to the apparatus, for example the one or more processors may be a remote processor (e.g., a cloud-based processor). Alternatively or additionally, the one or more processors may be internal and/or local to the apparatus. In this regard, a given circuit or components thereof may be disposed locally (e.g., as part of a local server, a local computing system, etc.) or remotely (e.g., as part of a remote server such as a cloud based server). To that end, a “circuit” as described herein may include components that are distributed across one or more locations.

An exemplary system for implementing the overall system or portions of the embodiments might include a computing system in the form of computers, including a processing unit, a system memory, and a system bus that couples various system components including the system memory to the processing unit. Each memory device may include non-transient volatile storage media, non-volatile storage media, non-transitory storage media (e.g., one or more volatile and/or non-volatile memories), a distributed ledger (e.g., a blockchain), etc. In some embodiments, the non-volatile media may take the form of ROM, flash memory (e.g., flash memory such as NAND, 3D NAND, NOR, 3D NOR, etc.), EEPROM, MRAM, magnetic storage, hard discs, optical discs, etc. In other embodiments, the volatile storage media may take the form of RAM, TRAM, ZRAM, etc. Combinations of the above are also included within the scope of machine-readable media. In this regard, machine-executable instructions comprise, for example, instructions and data that cause a general purpose computer, a special purpose computer, or special purpose processing machines to perform a certain function or group of functions. Each respective memory device may be operable to maintain or otherwise store information relating to the operations performed by one or more associated circuits, including processor instructions and related data (e.g., database components, object code components, script components, etc.), in accordance with the example embodiments described herein.

It should be understood that a “network interface,” as used herein, includes any of a cellular transceiver (e.g., Code Division Multiple Access (“CDMA”), Global System for Mobile Communications (“GSM”), Long-Term Evolution (“LTE”), etc.), a wireless network transceiver (e.g., 802.11X, ZigBee, or Bluetooth), an external network device (e.g., computer port, network interface card (“NIC”), network socket, port), or a combination thereof (e.g., both a cellular transceiver and a Bluetooth transceiver). In some arrangements, a network interface includes hardware and machine-readable media sufficient to support communication over multiple channels of data communication. Further, in some arrangements, the network interface includes cryptography capabilities to establish a secure, or relatively secure, communication session between one or more computing devices. In this regard, personal information about clients, medical records, financial data, and other types of data is encrypted and transmitted to prevent, or substantially prevent, the threat of hacking.

It should also be noted that the term “input devices” or “input components,” as described herein, may include any type of input device including, but not limited to, a keyboard, a keypad, a mouse, joystick or other input devices performing a similar function. Comparatively, the term “output device” or “output component,” as described herein, may include any type of output device including, but not limited to, a computer monitor, printer, facsimile machine, or other output devices performing a similar function.

It should be noted that although the diagrams herein may show a specific order and composition of method steps, it is understood that the order of these steps may differ from what is depicted. For example, two or more steps may be performed concurrently or with partial concurrence. Also, some method steps that are performed as discrete steps may be combined, steps being performed as a combined step may be separated into discrete steps, the sequence of certain processes may be reversed or otherwise varied, and the nature or number of discrete processes may be altered or varied. The order or sequence of any element or apparatus may be varied or substituted according to alternative embodiments. Accordingly, all such modifications are intended to be included within the scope of the present disclosure as defined in the appended claims. Such variations will depend on the machine-readable media and hardware systems chosen and on designer choice. It is understood that all such variations are within the scope of the disclosure. Likewise, software and web implementations of the present disclosure could be accomplished with standard programming techniques with rule-based logic and other logic to accomplish the various database searching steps, correlation steps, comparison steps and decision steps.

The foregoing description of embodiments has been presented for purposes of illustration and description. It is not intended to be exhaustive or to limit the disclosure to the precise form disclosed, and modifications and variations are possible in light of the above teachings or may be acquired from this disclosure. The embodiments were chosen and described in order to explain the principals of the disclosure and its practical application to enable one skilled in the art to utilize the various embodiments and with various modifications as are suited to the particular use contemplated. Other substitutions, modifications, changes and omissions may be made in the design, operating conditions and arrangement of the embodiments without departing from the scope of the present disclosure as expressed in the appended claims.

What is claimed is:

1. A mobile response unit, comprising:
a processing circuit including a processor and memory, the memory having instructions stored thereon that, when executed by the processor, cause the processor to:
establish a network to connect with a first user device, the first user device associated with a displacing facility;
receive, responsive to establishment of the network, from the first user device, a request to transfer a resident associated with the displacing facility;
retrieve, from the memory, a record associated with the resident;
retrieve, from the memory, information associated with a plurality of receiving facilities;
identify, based on the record associated with the resident and the information associated with the plurality of receiving facilities, a receiving facility of the plurality of receiving facilities to receive the resident;
establish, via the network, a connection with a second user device, the second user device associated with the receiving facility; and
transmit, via the network, to the second user device, one or more signals to cause the second user device to display a user interface including a prompt to accept the request to transfer the resident.

2. The mobile response unit of claim 1, wherein the instructions cause the processor to:
receive, via the network, from the second user device, an indication to accept the request to transfer the resident, wherein the indication is received responsive to a selection of a selectable element included in the prompt to accept the request to transfer the resident;
transmit, via the network, to the second user device, one or more second signals to cause the second user device to display, via the user interface, the record associated with the resident;
wherein the record associated with the resident is displayed without the second user device storing the record associated with the resident.

3. The mobile response unit of claim 2, wherein the instructions cause the processor to:
transmit, via the network, to the first user device, one or more third signals to cause the first user device to display a second user interface including a notice that the receiving facility accepted the request to transfer the resident;
receive, via the network, from the first user device, a second request for directions to the receiving facility;
generate, responsive to receipt of the second request, a route to the receiving facility;
transmit, via the network, to the first user device, one or more fourth signals to cause the first user device to display the route to the receiving facility; and
transmit, via the network, to the second user device, one or more fifth signals to cause the second user device to display, via the user interface, an indication that the resident is in route to the receiving facility.

4. The mobile response unit of claim 3, wherein the instructions cause the processor to:
receive, via the network, from a weather data source, a weather alert that includes information associated with a weather event;
determine, based on the weather event, that the route is impacted by the weather event; and
update, responsive to determination that the route is impacted by the weather event, the route to avoid the weather event.

5. The mobile response unit of claim 4, wherein the instructions cause the processor to:
transmit, via the network, to the first user device, one or more sixth signals to cause the first user device to display information associated with the weather event and to display an alert to indicate that the route has been updated, the alert including a prompt to accept the route;
receive, via the network, from the first user device, a response to indicate acceptance of the route; and
transmit, via the network, to the first user device, one or more seventh signals to cause the first user device to display the route.

6. The mobile response unit of claim 2, wherein the instructions cause the processor to:
identify, based on the record associated with the resident, a third user device;
establish, via the network, a connection with the third user device; and
transmit, via the network, one or more third signals to the cause the third user device to display an alert that the resident will be transferred to the receiving facility;
wherein the alert cause the third user device to display information associated with the receiving facility.

7. The mobile response unit of claim 6, wherein the instructions cause the processor to:
update, responsive to receipt of the indication to accept the request to transfer the resident, the record associated with the resident to reflect that the resident is being transferred to the receiving facility; and
display, via a display on the mobile response unit, a third user interface to indicate that the record associated with the resident has been updated to reflect that the resident is being transferred to the receiving facility.

8. The mobile response unit of claim 1, wherein the instructions cause the processor to:
receive, via the network, from the first user device, a request to associate the resident with a wearable record device;
establish, responsive to receipt of the request to associate the resident with the wearable record device, via the network, a connection with the wearable record device;
provide, via the network, to the wearable record device, the record associated with the resident to cause the wearable record device to store, in memory of the wearable record device, the record associated with the resident; and
receive, via the network, from the wearable record device, a confirmation to indicate that the resident is associated with the wearable record device.

9. The mobile response unit of claim 8, wherein the instructions cause the processor to:
control one or more pieces of medical equipment to perform a health check;
receive, responsive to performance of the health check, information associated with the health check; and
transmit, via the network, to the wearable record device, the information associated with the health check to cause the wearable record device to store, in the memory of the wearable record device, the information associated with the health check.

10. The mobile response unit of claim 1, wherein the information associated with the plurality of receiving facilities includes a distance from each receiving facility of the plurality of receiving facilities to the displacing facility, and wherein the receiving facility is identified responsive to the distance from the receiving facility to the displacing facility being within a predetermined threshold.

11. The mobile response unit of claim 10, wherein the information associated with the plurality of receiving facilities further includes at least one of:
a number of available beds for each receiving facility of the plurality of facilities; or
a number of staff for each receiving facility of the plurality of facilities.

12. The mobile response unit of claim 1, wherein the instructions cause the processor to receive, from the first user device, the request to transfer the resident associated with the displacing facility by:
transmitting, via the network, to the first user device, one or more second signals to cause the first user device to display a list including a plurality of residents associated with the displacing facility, the plurality of residents including the resident;
receiving, via the network, from the first user device, an indication of a selection of a selectable element associated with the resident;
transmitting, via the network, to the first user device, responsive to receipt of the indication of the selection of the selectable element, one or more third signals to cause the first user device to display information associated with the resident; and
receive, via the network, from the first user device the request to transfer the resident.

13. A mobile response unit, comprising:
a processing circuit including a processor and memory, the memory having instructions stored thereon that, when executed by the processor, cause the processor to:
establish a network to connect with a first user device, the first user device associated with a displacing facility;
receive, responsive to establishment of the network, from the first user device, a request to transfer a resident associated with the displacing facility;
retrieve, from the memory, a record associated with the resident;
retrieve, from the memory, information associated with a plurality of receiving facilities;
identify, based on the record associated with the resident and the information associated with the plurality of receiving facilities, a receiving facility of the plurality of receiving facilities to receive the resident;
establish, via the network, a connection with a second user device, the second user device associated with the receiving facility;
transmit, via the network, to the second user device, one or more signals to cause the second user device to display a user interface including a prompt to accept the request to transfer the resident;
receive, via the network, from the second user device, an indication to accept the request to transfer the resident, wherein the indication is received responsive to a selection of a selectable element included in the prompt to accept the request to transfer the resident;
transmit, via the network, to the second user device, one or more second signals to cause the second user device to display, via the user interface, the record associated with the resident;
wherein the record associated with the resident is displayed without the second user device storing the record associated with the resident.

14. The mobile response unit of claim 13, wherein the instructions cause the processor to:
transmit, via the network, to the first user device, one or more third signals to cause the first user device to display a second user interface including a notice that the receiving facility accepted the request to transfer the resident;
receive, via the network, from the first user device, a second request for directions to the receiving facility;
generate, responsive to receipt of the second request, a route to the receiving facility;
transmit, via the network, to the first user device, one or more fourth signals to cause the first user device to display the route to the receiving facility; and
transmit, via the network, to the second user device, one or more fifth signals to cause the second user device to display, via the user interface, an indication that the resident is in route to the receiving facility.

15. The mobile response unit of claim 14, wherein the instructions cause the processor to:
receive, via the network, from a weather data source, a weather alert that includes information associated with a weather event;
determine, based on the weather event, that the route is impacted by the weather event;
update, responsive to determination that the route is impacted by the weather event, the route to avoid the weather event;
transmit, via the network, to the first user device, one or more sixth signals to cause the first user device to display information associated with the weather event and to display an alert to indicate that the route has been updated, the alert including a prompt to accept the route;
receive, via the network, from the first user device, a response to indicate acceptance of the route; and
transmit, via the network, to the first user device, one or more seventh signals to cause the first user device to display the route.

16. The mobile response unit of claim 13, wherein the instructions cause the processor to:
identify, based on the record associated with the resident, a third user device;
establish, via the network, a connection with the third user device; and
transmit, via the network, one or more third signals to the cause the third user device to display an alert that the resident will be transferred to the receiving facility;
wherein the alert cause the third user device to display information associated with the receiving facility.

17. The mobile response unit of claim 16, wherein the instructions cause the processor to:
update, responsive to receipt of the indication to accept the request to transfer the resident, the record associated with the resident to reflect that the resident is being transferred to the receiving facility; and
display, via a display on the mobile response unit, a third user interface to indicate that the record associated with the resident has been updated to reflect that the resident is being transferred to the receiving facility.

18. The mobile response unit of claim 13, wherein the instructions cause the processor to:
receive, via the network, from the first user device, a request to associate the resident with a wearable record device;
establish, responsive to receipt of the request to associate the resident with the wearable record device, via the network, a connection with the wearable record device;
provide, via the network, to the wearable record device, the record associated with the resident to cause the wearable record device to store, in memory of the wearable record device, the record associated with the resident; and
receive, via the network, from the wearable record device, a confirmation to indicate that the resident is associated with the wearable record device.

19. The mobile response unit of claim 18, wherein the instructions cause the processor to:
control one or more pieces of medical equipment to perform a health check;
receive, responsive to performance of the health check, information associated with the health check; and
transmit, via the network, to the wearable record device, the information associated with the health check to cause the wearable record device to store, in the memory of the wearable record device, the information associated with the health check.

20. A mobile response unit, comprising:
a processing circuit including a processor and memory, the memory having instructions stored thereon that, when executed by the processor, cause the processor to:
establish a network to connect with a first user device, the first user device associated with a displacing facility;
receive, responsive to establishment of the network, from the first user device, a request to transfer a resident associated with the displacing facility;
retrieve, from the memory, a record associated with the resident;
retrieve, from the memory, information associated with a plurality of receiving facilities;
identify, based on the record associated with the resident and the information associated with the plurality of receiving facilities, a receiving facility of the plurality of receiving facilities to receive the resident;
establish, via the network, a connection with a second user device, the second user device associated with the receiving facility;
transmit, via the network, to the second user device, one or more signals to cause the second user device to display a user interface including a prompt to accept the request to transfer the resident;
receive, via the network, from the first user device, a request to associate the resident with a wearable record device;
establish, responsive to receipt of the request to associate the resident with the wearable record device, via the network, a connection with the wearable record device;
provide, via the network, to the wearable record device, the record associated with the resident to cause the wearable record device to store, in memory of the wearable record device, the record associated with the resident; and
receive, via the network, from the wearable record device, a confirmation to indicate that the resident is associated with the wearable record device.
